(19) United States
(12) Patent Application Publication
(10) Pub. No.: US 2011/0224197 A1
(43) Pub. Date: Sep. 15, 2011

(54) PYRIMIDINES AND PYRIDINES USEFUL AS INHIBITORS OF PROTEIN KINASES

(75) Inventors: Gregory William Henkel, Carlsbad, CA (US); Michael Liu, Oceanside, CA (US); Timothy Neuberger, San Diego, CA (US)

(73) Assignee: Vertex Pharmaceuticals Incorporated, Cambridge, MA (US)

(21) Appl. No.: 12/878,246

(22) Filed: Sep. 9, 2010

Related U.S. Application Data
(63) Continuation of application No. PCT/US2009/001534, filed on Mar. 10, 2009.
(60) Provisional application No. 61/035,290, filed on Mar. 10, 2008.

(51) Int. Cl. A61K 31/541; A61K 31/517; A61K 31/506; A61K 31/5377; A61K 31/496; A61K 31/4439; A61K 31/444; C12N 9/99; C12Q 1/48; C07D 471/04; C07D 417/14; C07D 495/04; C07D 403/12; A61P 25/00; A61P 25/28; A61P 25/16; A61P 19/10; A61P 7/00; A61P 3/10; A61P 19/02; A61P 25/18; A61P 9/00

(52) U.S. Cl. 514/228.2; 514/258.1; 514/256; 514/266.23; 514/234.5; 514/252.19; 514/342; 514/303; 435/184; 435/15; 544/279; 544/62; 544/278; 544/328; 544/295

(57) ABSTRACT

The present invention relates to compounds useful as inhibitors of protein kinases, such as inhibitors of GSK-3. The invention provides pharmaceutically acceptable compositions comprising the compounds of the invention and methods of using the compositions in the treatment of various disorders. The invention provides processes for preparing compounds of the invention. The invention provides methods of identifying compounds useful for treatment of diabetes, diabetic neuropathy, osteoporosis, Alzheimer's disease, Huntington's disease, Parkinson's disease, AIDS-associated dementia, bipolar disorder, amyotrophic lateral sclerosis, multiple sclerosis, schizophrenia, leukocytopenia, cardiomyocyte hypertrophy, stroke, post-stroke, spinal cord injury, traumatic brain injury, Charcot-Marie-Tooth, peripheral nerve regeneration, and rheumatoid arthritis.

### CROSS-REFERENCE TO RELATED APPLICATIONS

This application claims the benefit of U.S. patent application 61/035,290 filed Mar. 10, 2008, the contents of which are incorporated herein in their entirety.

### TECHNICAL FIELD OF THE INVENTION

The present invention relates to compounds useful as inhibitors of protein kinases. The invention also provides pharmaceutically acceptable compositions comprising the compounds of the invention and methods of using the compositions in the treatment of various disorders. The invention also provides processes for preparing the compounds of the invention. The invention also provides methods of identifying compounds which are useful for the treatment of a number of disorders including diabetes, diabetic neuropathy, osteoporosis, Alzheimer's disease, Huntington's disease, Parkinson's disease, AIDS-associated dementia, bipolar disorder, amyotrophic lateral sclerosis (ALS, Lou Gehrig's disease), multiple sclerosis (MS), schizophrenia, leukocytopenia, cardiomyocyte hypertrophy, stroke, post-stroke, spinal cord injury, traumatic brain injury, Charcot-Marie-Tooth, peripheral nerve regeneration, and rheumatoid arthritis.

### BACKGROUND OF THE INVENTION

Glycogen synthase kinase-3 (GSK-3) is a serine/threonine protein kinase comprised of α and β isoforms that are each encoded by distinct genes. (Coghlan et al., Chemistry & Biology 2000, 7, 793-803; and Kim and Kimmel, Curr. Opinion Genetics Dev., 2000 10, 508-514).

GSK-3 has been implicated in various diseases, disorders, and conditions. GSK-3 regulates multiple downstream effectors associated with a variety of signaling pathways. These proteins include glycogen synthase, which is the rate limiting enzyme necessary for glycogen synthesis, the microtubule associated protein Tau, the gene transcription factor β-catenin, the translation initiation factor e1F2B, as well as ATP citrate lyase, axin, heat shock factor-1, c-Jun, c-myc, c-myb, CREB, and CEPBα. These diverse protein targets implicate GSK-3 in many aspects of cellular metabolism, proliferation, differentiation, and development.

GSK-3 functions as both a tyrosine and a serine/threonine kinase, similar to the DYRK kinase family. Like the DYRK kinase family, GSK-3 auto-phosphorylates a tyrosine residue in its kinase domain (GSK-3a, Tyr 279 and GSK-3b, Tyr 216). This tyrosine phosphorylation has been shown to be important for positively modulating kinase activity. Locheed et al., demonstrated that this autophosphorylation occurs intra-molecularly at a post-translationally intermediate step prior to maturation and is chaperone dependent (Lochhead et al., Molecular Cell 24, 2006, pp. 627-633). After maturation, GSK-3 loses its tyrosine kinase activity and acts exclusively as a serine and threonine kinase towards exogenous substrates.

β-catenin is one of the exogenous serine/threonine substrates that GSK-3 phosphorylates. As primarily a serine/threonine kinase, GSK-3 is central to many signalling pathways that control multiple cellular activities such as proliferation, differentiation and metabolism. Because GSK-3 plays a central role in multiple signaling pathways, there is a need for compounds that can partially attenuate GSK-3 activity without completely blocking the enzyme and affecting multiple substrates, such as p-catenin.

There is a great need to develop compounds useful as inhibitors of protein kinases. In particular, it would be desirable to develop compounds that are useful as inhibitors of GSK-3, particularly given the inadequate treatments currently available for the majority of the disorders implicated in GSK-3 activation.

### SUMMARY OF THE INVENTION

In one aspect, the invention features a method of treating a GSK-3 mediated condition comprising administering a therapeutically effective amount of a compound of formula I:

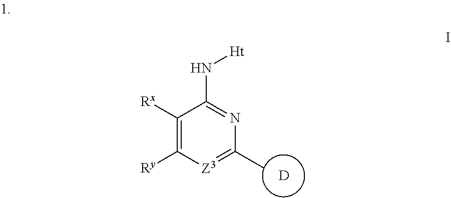

or a pharmaceutically acceptable salt thereof, wherein:

Ht is

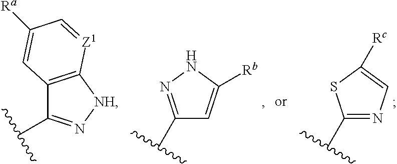

Ring D is phenyl, a 3-8 membered monocyclic cycloalophatic, a 5-8 membered monocyclic heterocycloaliphatic containing 1-2 heteroatoms and bound to the pyridine or pyrimidine ring via a carbon ring atom, adamantyl, or an 8-10 membered bicyclic cycloaliphatic, wherein the phenyl, heterocycloaliphtic, monocyclic, bicyclic or cycloaliphatic is optionally substituted with 1-2 of —R⁵;

R^{a} is H or halogen;

R^{b} is H or C₁₋₄alkyl;

R^{c} is H or C₁₋₄alkyl;

Z¹ is N or CH;

Z³ is N or CR^{Z};

R^{X} is H or C₁₋₄alkyl;

R^{Y} is H, halogen, a 4-8 membered monocyclic non-aromatic heterocyclyl optionally substituted with one R¹⁰, or C₁₋₄alkyl optionally substituted with NR₁R₂, 1-3 halo, —OR, or a 4-8 membered monocyclic non-aromatic heterocyclyl containing 1-2 heteroatoms selected from O, N, or S and being optionally substituted with —R¹⁰, or
R^{X} and R^{Y} together with the atoms to which they are bound form phenyl, a 6 to 8 membered cycloaliphatic, or a 5-8 membered monocyclic heterocyclyl containing 1-2 heteroatoms selected from O, N, or S;

R^{Z} is H or C₁₋₄ alkyl;

R¹ is H or C₁₋₄ alkyl;

R² is H or C₁₋₄ alkyl optionally substituted with —R¹¹;

Each R⁵ is independently C₁₋₆ alkyl, haloC₁₋₆alkyl, or halo;

Each R¹⁰ is independently selected from C₁₋₆ alkyl, haloC₁₋₆alkyl, halo, OR, C(═O)R, CO₂R, S(O)R, SO₂R, SR, N(R⁴)₂, CON(R⁴)₂, SO₂N(R⁴)₂, OC(═O)R, N(R⁴)COR, or N(R⁴)CO₂R;

Each R¹¹ is independently selected from halo, OR, C(═O)R, CO₂R, N(R⁴)₂, CON(R⁴)₂, OC(═O)R, N(R⁴)COR, or N(R⁴)CO₂R;

Each R⁴ is independently selected from H, C₁₋₆alkyl, or haloC₁₋₆alkyl; and

Each R is independently selected from H, C₁₋₆alkyl, or haloC₁₋₆alkyl.

In an embodiment, Ht is

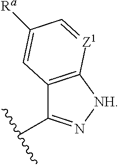

In other embodiments, Z¹ is N, or CH; R^{a} is halogen or F.

In another embodiment, Ht is

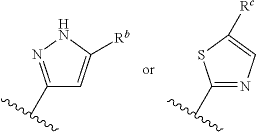

In other embodiments, R⁶ is H or CH₃.

In additional embodiments, ring D is phenyl optionally substituted with —R⁵. In addition the phenyl can be substituted ortho relative to the attachment to the pyridine or pyrimidine ring. The phenyl can also be substituted with halogen, including Cl. The phenyl can also be substituted with C₁₋₆ alkyl, including CH₃. Furthermore the phenyl can be substituted with haloC₁₋₆ alkyl, including CF₃.

In other embodiments, ring D can also be a 3-8 membered monocyclic or 8-10 membered bicyclic cycloaliphatic, wherein cycloaliphatic is optionally substituted with —R⁵. Additional embodiments include Ring D being a 3-8 membered monocyclic cycloaliphatic and an 8-10 membered bicyclic cycloaliphatic.

In further embodiments, ring D is a 5-8 membered monocyclic heterocyclic, including tetrahydropyranyl. In other embodiments, R^{Y} is C₁₋₄alkyl optionally substituted with NR₁R₂ or a 4-8 membered monocyclic non-aromatic heterocyclyl optionally substituted with —R¹⁰. R^{Y} can also be CH₃. R^{Y} can also be ethyl optionally substituted with NR₁R₂ or a 4-8 membered monocyclic non-aromatic heterocyclyl optionally substituted with —R¹⁰. In additional embodiments, R^{Y} is —CH₂—CH₂—NR₁R₂ or —CH₂—CH₂-(4-8 membered monocyclic non-aromatic heterocyclyl) optionally substituted with —R¹⁰.

In other embodiments, R^{X} is H or CH₃.

In still other embodiments, R^{X} and R^{Y} together with the atoms to which they are bound form phenyl, a 6 to 8 membered cycloaliphatic or a 5 to 8 membered heterocycle.

In still yet other embodiments, the compound is selected from compounds I-1 through I-55.

In further embodiments, the GSK-3 mediated condition is treated by inhibiting the GSK 3 activity in an ex vivo or in vitro biological sample.

In additional embodiments, the GSK-3 mediated condition is selected from diabetes, osteoporosis, Alzheimer's disease, Huntington's disease, Parkinson's disease, AIDS-associated dementia, bipolar disorder, amyotrophic lateral sclerosis (ALS, Lou Gehrig's disease), multiple sclerosis (MS), schizophrenia, leukocytopenia, stroke, neurological disorders, spinal cord injury, traumatic brain injury, rheumatoid arthritis, Charcot-Marie-Tooth, peripheral nerve regeneration, and diabetic neuropathy.

In a further embodiment, the compound is administered after ischemia has occurred.

In yet another embodiment, the method comprises the additional step of administering to said patient an additional therapeutic agent selected from an agent for treating diabetes, agent for treating osteoporosis, an agent for treating Alzheimer's disease, an agent for treating Huntington's disease, an agent for treating Parkinson's disease, an agent for treating AIDS-associated dementia, an agent for treating bipolar disorder, an agent for treating amyotrophic lateral sclerosis (ALS, Lou Gehrig's disease), an agent for treating multiple sclerosis (MS), an agent for treating schizophrenia, an agent for treating leukocytopenia, an agent for treating peripheral nerve regeneration, an agent for treating stroke, treating spinal cord injury, an agent for treating traumatic brain injury, an agent for treating Charcot-Marie-Tooth, an agent for treating diabetic neuropathy and an agent for treating rheumatoid arthritis, wherein the additional therapeutic agent is appropriate for the disease being treated; and the additional therapeutic agent is administered together with said composition as a single dosage form or separately from said composition as part of a multiple dosage form.

In another aspect, the invention features a method for treating a GSK-3 mediated condition comprising administering an agent that selectively inhibits the auto-phosphorylation of the tyrosine form of the GSK-3 enzyme relative to the serine/threonine kinase form. In one embodiment, the agent is a compound as described above. In another embodiment, the GSK-3 mediated condition is Post-Stroke, Spinal Cord Injury, Traumatic Brain Injury, Alzheimer's disease, Parkinson's disease, Huntington's disease, Multiple Sclerosis, Amyotrophic Lateral Sclerosis, Diabetic Neuropathy, Charcot-Marie-Tooth, Leukocytopenia, Diabetes, peripheral nerve regeneration or Osteoporosis.

In yet another aspect, the invention features a the method of increasing axonal and dendritic branching in neuronal cells, comprising administering an agent that selectively inhibits the auto-phosphorylation of the tyrosine form of the GSK-3 enzyme relative to the serine/threonine kinase form. In one embodiment, the agent is a compound as described above.

In still yet another aspect, the invention features a method of promoting neuroplasticity comprising, administering an agent that selectively inhibits the auto-phosphorylation of the tyrosine form of the GSK-3 enzyme relative to the serine/threonine kinase form. In one embodiment, the agent is a compound as described above.

In a further aspect, the invention features a method of promoting angiogenesis comprising, administering an agent that selectively inhibits the auto-phosphorylation of the tyrosine form of the GSK-3 enzyme relative to the serine/threonine kinase form. In one embodiment, the agent is a compound as described above.

In another aspect, the invention features a method of promoting neurogenesis comprising administering an agent that selectively inhibits the auto-phosphorylation of the tyrosine form of the GSK-3 enzyme relative to the serine/threonine kinase form. In one embodiment, the agent is a compound as described in above.

In yet another aspect, the invention features a method of treating neuropsychiatric disorders comprising administering an agent that selectively inhibits the auto-phosphorylation of the tyrosine form of the GSK-3 enzyme relative to the serine/threonine kinase form. In one embodiment, the agent is a compound as described above. In another embodiment, the neuropsychiatric disorder is mania or depression.

In still another aspect, the invention features a method for identifying compounds useful for the treatment of GSK-3-mediated conditions comprising measuring the amount of auto-phosphorylation of the tyrosine of the GSK-3 enzyme relative to the serine/threonine kinase form for one or more test compounds. In an embodiment, the method comprises measuring the amount of auto-phosphorylation of the tyrosine of the GSK-3 enzyme and measuring the amount of phosphorylation oβ-catenin. In another embodiment, the step of measuring comprises the (3-catenin IC50 value for the test compound, determining the GSK-3α or GSK3β p-TYR IC50 value, and dividing the β-catenin IC50 value by the GSK-3α or GSK3β p-TYR IC50 value.

In still yet another aspect, the invention features a method for identifying compounds useful for increasing axonal and dendritic branching in neuronal cells comprising measuring the amount of auto-phosphorylation of the tyrosine form of the GSK-3 enzyme relative to the serine/threonine kinase form for one or more test compounds. In an embodiment, the method comprises measuring the amount of auto-phosphorylation of the tyrosine of the GSK-3 enzyme and measuring the amount of phosphorylation of (β-catenin. In another embodiment, the step of measuring comprises the β-catenin IC50 value for the test compound, determining the GSK-3α or GSK3β p-TYR IC50 value, and dividing the β-catenin IC50 value by the GSK-3α or GSK3β p-TYR IC50 value. In yet another embodiment, the method further comprises identifying compounds which exhibit a ratio of β-catenin IC50 to GSK-3α or GSK3β p-TYR IC50 of about 10 or higher or 30 or higher.

In a further aspect, the invention features a method of providing post-stroke recovery, comprising administering an agent that selectively inhibits the auto-phosphorylation of the tyrosine form of the GSK-3 enzyme relative to the serine/threonine kinase form. In one embodiment, the agent is compound as described above. In another embodiment, the agent is administered during or immediately after ischemia. The agent can also be administered during or immediately after ischemia and for a period of about 6 months after ischemia. In yet another embodiment, physical therapy is also administered.

In another aspect, the invention features a compound that is selected from compound I-39 through compound I-55.

Advantageously and unexpectedly, compounds that selectively inhibit the auto-phosphorylation of the tyrosine form of the GSK-3 enzyme relative to the serine/threonine kinase form result in increased neuron growth and dendrite formation. Increases in neuron growth and dendrite formation are advantageous when treating many types of degenerative conditions such as stroke, post-stroke recovery, Alzheimer's Disease, Parkinson's Disease, Huntington's Disease, Amyotrophic Lateral Sclerosis (ALS) Multiple Sclerosis (MS), Spinal Cord Injury, Traumatic Brain Injury, Charcot-Marie-Tooth, Leukocytopenia, Diabetes, Diabetic Neuropathy, Peripheral Nerve Regeneration and Osteoporosis.

### DETAILED DESCRIPTION OF THE INVENTION

### Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As described herein, a specified number range of atoms includes any integer therein. For example, a group having from 1-4 atoms could have 1, 2, 3, or 4 atoms.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable”, as used herein, refers to compounds that are not substantially altered when subjected to conditions to allow for their production, detection, recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched) or cyclic, branched or unbranched, substituted or unsubstituted hydrocarbon chain that is completely saturated or that contains one or more units of unsaturation that has a single point of attachment to the rest of the molecule. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. Suitable aliphatic groups include, but are not limited to, linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl.

The term “alkyl” as used herein, means a branched or unbranched, substituted or unsubstituted, hydrocarbon chain that is completely saturated and has a single point of attachment to the rest of the molecule. Unless otherwise specified, alkyl groups contain 1-6 alkyl carbon atoms. In some embodiments, alkyl groups contain 1-4 alkyl carbon atoms. In other embodiments, alkyl groups contain 1-3 alkyl carbon atoms. Examples include, but are not limited to, methyl, ethyl, isopropyl, n-propyl, sec-butyl, n-butyl, and n-pentyl.

The term “cycloaliphatic” (or “carbocycle” or “carbocyclyl” or “cycloalkyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable cycloaliphatic groups include, but are not limited to, cycloalkyl and cycloalkenyl groups. Specific examples include, but are not limited to, cyclohexyl, cyclopropenyl, and cyclobutyl.

The term “heteroaliphatic”, as used herein, means aliphatic groups wherein one or two carbon atoms are independently replaced by one or more of oxygen, sulfur, nitrogen, phosphorus, or silicon. Heteroaliphatic groups may be substituted or unsubstituted, branched or unbranched, cyclic or acyclic, and include “heterocycle”, “heterocyclyl”, “heterocycloaliphatic”, or “heterocyclic” groups.

The term “heterocycle”, “heterocyclyl”, or “heterocyclic” as used herein means non-aromatic, monocyclic, bicyclic, or tricyclic ring systems in which one or more ring members are an independently selected heteroatom. In some embodiments, the “heterocycle”, “heterocyclyl”, or “heterocyclic” group has three to fourteen ring members in which one or more ring members is a heteroatom independently selected from oxygen, sulfur, nitrogen, or phosphorus, and each ring in the system contains 3 to 7 ring members.

Suitable heterocycles include, but are not limited to, 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydroimidazol-2-one.

Cyclic groups, (e.g. cycloaliphatic and heterocycles), can be linearly fused, bridged, or spirocyclic.

The term “heteroatom” means one or more of oxygen, sulfur, nitrogen, or phosphorus, (including, any oxidized form of nitrogen, sulfur, or phosphorus; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

The term “alkoxy”, or “thioalkyl”, as used herein, refers to an alkyl group, as previously defined, attached to the principal carbon chain through an oxygen (“alkoxy”) or sulfur (“thioalkyl”) atom.

The terms “haloalkyl”, “haloalkenyl”, “haloaliphatic”, and “haloalkoxy” mean alkyl, alkenyl or alkoxy, as the case may be, substituted with one or more halogen atoms. The terms “halogen”, “halo”, and “hal” mean F, Cl, Br, or I.

The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring”. The term “aryl” also refers to heteroaryl ring systems as defined hereinbelow.

The term “heteroaryl”, used alone or as part of a larger moiety as in “heteroaralkyl” or “heteroarylalkoxy”, refers to monocyclic, bicyclic, or tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”. Suitable heteroaryl rings include, but are not limited to, 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “protecting group” and “protective group” as used herein, are interchangeable and refer to an agent used to temporarily block one or more desired reactive sites in a multifunctional compound. In certain embodiments, a protecting group has one or more, or preferably all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. Exemplary protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

In some embodiments, an alkyl or aliphatic chain can be optionally replaced with another atom or group. Examples of such atoms or groups would include, but are not limited to, —NR—, —O—, —S—, —CO₂—, —OC(O)—, —C(O)CO—, —C(O)—, —C(O)NR—, —C(═N—CN), —NRCO—, —NRC(O)O—, —SO₂NR—, —NRSO₂—, —NRC(O)NR—, —OC(O)NR—, —NRSO₂NR—, —SO—, or —SO₂—, wherein R is defined herein. Unless otherwise specified, the optional replacements form a chemically stable compound. Optional replacements can occur both within the chain and at either end of the chain; i.e. both at the point of attachment and/or also at the terminal end. Two optional replacements can also be adjacent to each other within a chain so long as it results in a chemically stable compound. The optional replacements can also completely replace all of the carbon atoms in a chain. For example, a C₃ aliphatic can be optionally interrupted or replaced by —NR—, —C(O)—, and —NR— to form —NRC(O)NR— (a urea).

Unless otherwise specified, if the replacement occurs at the terminal end, the replacement atom is bound to an H on the terminal end. For example, if —CH₂CH₂CH₃ were optionally replaced with —O—, the resulting compound could be —OCH₂CH₃, —CH₂OCH₃, or —CH₂CH₂OH.

Unless otherwise indicated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise indicated, all tautomeric forms of the compounds of the invention are within the scope of the invention.

Unless otherwise indicated, a substituent can freely rotate around any rotatable bonds. For example, a substituent drawn as

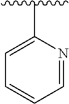

also represents

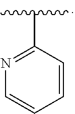

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

It will also be appreciated that the compounds of the present invention can exist in free form for treatment, or where appropriate, as a pharmaceutically acceptable salt, salts, or mixtures thereof.

As used herein, the term “pharmaceutically acceptable salt” refers to salts of a compound which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4alkyl)₄ salts. This invention also envisions the quaternization of any basic nitrogen-containing groups of the compounds disclosed herein. Water or oil-soluble or dispersible products may be obtained by such quaternization.

Base addition salts can be prepared by 1) reacting the purified compound in its acid form with a suitable organic or inorganic base and 2) isolating the salt thus formed. Base addition salts include alkali or alkaline earth metal salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

Compounds

Compounds useful for treating GSK-3 mediated condition include those of formula I

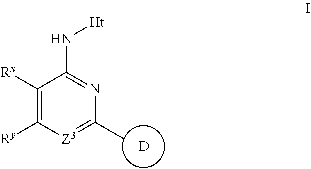

or a pharmaceutically acceptable salt thereof, wherein:

Ht is

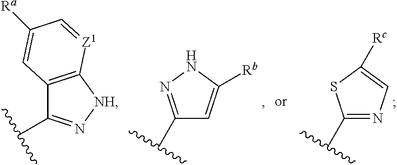

Ring D is phenyl, a 3-8 membered monocyclic cycloalophatic, a 5-8 membered monocyclic heterocycloaliphatic containing 1-2 heteroatoms and bound to the pyridine or pyrimidine ring via a carbon ring atom, adamantyl, or an 8-10 membered bicyclic cycloaliphatic, wherein the phenyl, heterocycloaliphtic, monocyclic, bicyclic or cycloaliphatic is optionally substituted with 1-2 of —R⁵;
R^{a} is H or halogen;
R^{b} is H or C₁₋₄alkyl;
R^{c} is H or C₁₋₄alkyl;
Z¹ is N or CH;
Z³ is N or CR^{Z};
R^{X} is H or C₁₋₄alkyl;
R^{Y} is H, halogen, a 4-8 membered monocyclic non-aromatic heterocyclyl optionally substituted with one R¹⁰, or C₁₋₄alkyl optionally substituted with NR₁R₂, 1-3 halo, —OR, or a 4-8 membered monocyclic non-aromatic heterocyclyl containing 1-2 heteroatoms selected from O, N, or S and being optionally substituted with —R¹⁰, or
   R^{X} and R^{Y} together with the atoms to which they are bound form phenyl, a 6 to 8 membered cycloaliphatic, or a 5-8 membered monocyclic heterocyclyl containing 1-2 heteroatoms selected from O, N, or S;
R^{Z} is H or C₁₋₄ alkyl;
R¹ is H or C₁₋₄ alkyl;
R² is H or C₁₋₄ alkyl optionally substituted with —R¹¹;
Each R⁵ is independently C₁ alkyl, haloC₁₋₆allyl, or halo;
Each R¹⁰ is independently selected from C₁₋₆alkyl, haloC₁₋₆alkyl, halo, OR, C(═O)R, CO₂R, S(O)R, SO₂R, SR, N(R⁴)₂, CON(R⁴)₂, SO₂N(R⁴)₂, OC(═O)R, N(R⁴)COR, or N(R⁴)CO₂R;
Each R¹¹ is independently selected from halo, OR, C(═O)R, CO₂R, N(R⁴)₂, CON(R⁴)₂, OC(═O)R, N(R⁴)COR, or N(R⁴)CO₂R;
Each R⁴ is independently selected from H, C₁₋₆alkyl, or haloC₁₋₆allyl; and
Each R is independently selected from H, C₁₋₆allyl, or haloC₁₋₆alkyl.

Embodiments of the compound of formula I may include one or more of the following specific features.

### Embodiments of Ht

Ht is

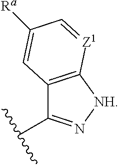

### Ht is

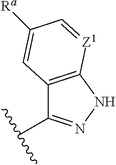

### and Z¹ is N. Ht is

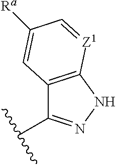

### and Z¹ is CH. Ht is

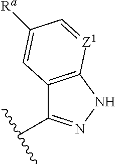

and R^{a} is halogen, such as F. Ht is

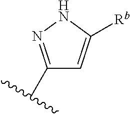

### and R^{b} is H or CH₃. Ht is

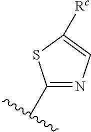

### and R^{c} is H or CH₃.

### Embodiments of Ring D

Ring D is phenyl optionally substituted with —R⁵. Ring D is phenyl substituted with one —R⁵. Ring D is phenyl is substituted ortho relative to the attachment to the pyridine or pyrimidine ring. Ring D is phenyl substituted with halogen, such as Cl. Ring D is 18. The method of claims 13-15, wherein the phenyl is substituted with C₁₋₆ alkyl, ring D is phenyl substituted with CH₃. Ring D is phenyl substituted with haloC₁₋₆ alkyl, such as CF₃. Ring D is a 3-8 membered monocyclic or 8-10 membered bicyclic cycloaliphatic, wherein the cycloaliphatic is optionally substituted with —R⁵. Ring D is a 3-8 membered monocyclic cycloaliphatic. Ring D is a 8-10 membered bicyclic cycloaliphatic. Ring D is a 5-8 membered monocyclic heterocyclic. Ring D is tetrahydropyranyl. Ring D is adamantyl.

### Embodiments of R^{Y}

R^{Y} is C₁₋₄alkyl optionally substituted with NR₁R₂. R^{Y} is C₁₋₄alkyl optionally substituted with a 4-8 membered monocyclic non-aromatic heterocyclyl optionally substituted with —R¹⁰. R^{Y} is CH₃. R^{Y} is ethyl optionally substituted with NR₁R₂. R^{Y} is a 4-8 membered monocyclic non-aromatic heterocyclyl optionally substituted with —R¹⁰. R^{Y} is —CH₂—CH₂—NR₁R₂. R^{Y} is —CH₂—CH₂-(4-8 membered monocyclic non-aromatic heterocyclyl) optionally substituted with —R¹⁰. R^{Y} is C₁₋₄alkyl optionally substituted with NR₁R₂ and R₂ is C₁₋₄alkyl optionally substituted with C(═O)R, CO₂R, N(R⁴)₂, or CON(R⁴)₂.

### Embodiments of R^{X}

R^{X} is H. R^{X} is CH₃.

### Embodiments of R^{X} and R^{Y}

R^{X} and R^{Y} together with the atoms to which they are bound form phenyl. R^{X} and R^{Y} together with the atoms to which they are bound form a 6 to 8 membered cycloaliphatic. R^{X} and R^{Y} together with the atoms to which they are bound form a 5 to 8 membered heterocycle.

In other embodiments, compounds useful for treating a GSK-3 mediated condition formula Ia

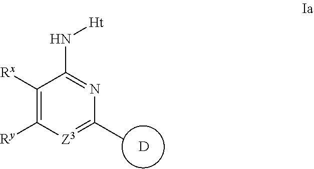

or a pharmaceutically acceptable salt thereof, wherein:

Ht is

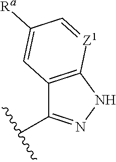

Ring D is phenyl substituted ortho relative to the point of attachment of the phenyl ring to the pyrimidine or pyridine ring of formula Ia with R⁵;
R^{a} is H or halogen;
R^{b} is H or C₁₋₄alkyl;
R^{c} is H or C₁₋₄alkyl;
Z¹ is N or CH;
Z³ is N or CR^{Z};
R^{X} is H or C₁₋₄alkyl;
R^{Y} is H, halogen, or C₁₋₄alkyl optionally substituted with NR₁R₂ or a 4-8 membered monocyclic non-aromatic heterocyclyl; where said heterocyclyl contains 1-2 heteroatoms selected from O, N, or S; and wherein the heterocyclyl is optionally substituted with —R¹⁰;
R^{Z} is H or C₁₋₄ alkyl;
R¹ is H or C₁₋₄ alkyl;
R² is H or C₁ alkyl optionally substituted with —R¹¹;
Each R⁵ is independently C₁₋₆ alkyl, haloC₁₋₆alkyl, or halo;
Each R¹⁰ is independently selected from C₁₋₆ alkyl, haloC₁₋₆alkyl, halo, OR, C(═O)R, CO₂R, S(O)R, SO₂R, SR, N(R⁴)₂, CON(R⁴)₂, SO₂N(R⁴)₂, OC(═O)R, N(R⁴)COR, or N(R⁴)CO₂R;
Each R¹¹ is independently selected from halo, OR, C(═O)R, CO₂R, N(R⁴)₂, CON(R⁴)₂, OC(═O)R, N(R⁴)COR, or N(R⁴)CO₂R;
Each R⁴ is independently selected from H, C₁₋₆alkyl, or haloC₁₋₆alkyl; and
Each R is independently selected from H, C₁₋₆alkyl, or haloC₁₋₆alkyl.

Specific, non-limiting examples of the compounds useful for treating a GSK-3 mediated condition are provided in Table 1.

TABLE 1

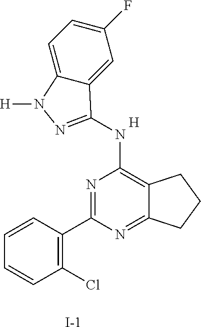

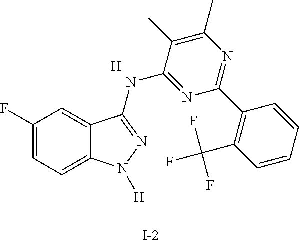

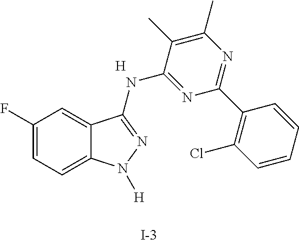

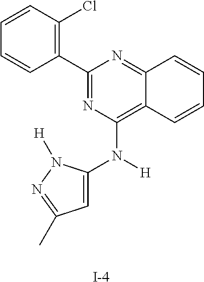

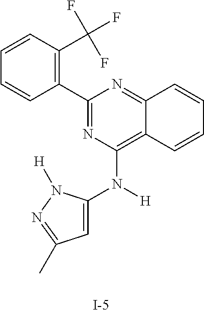

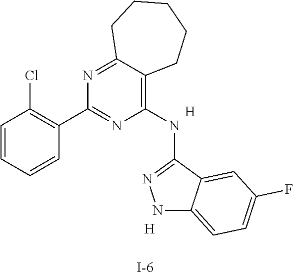

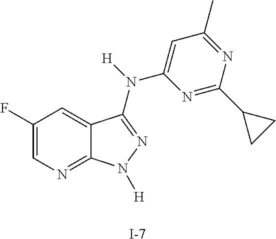

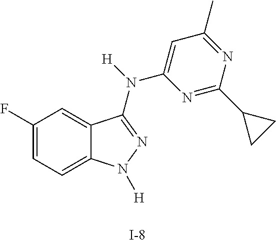

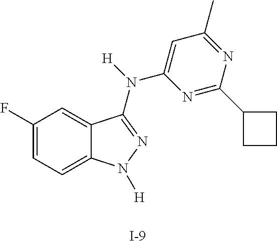

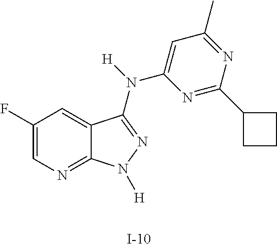

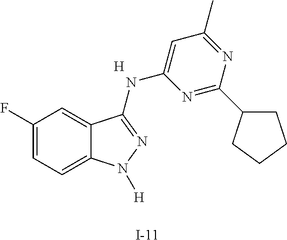

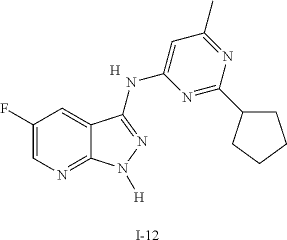

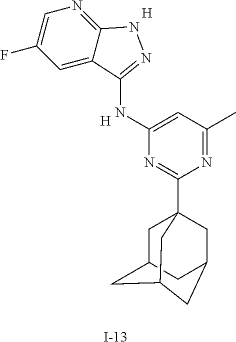

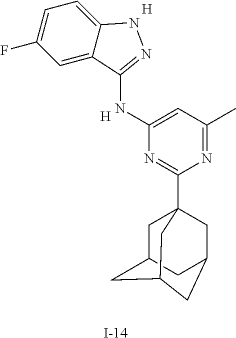

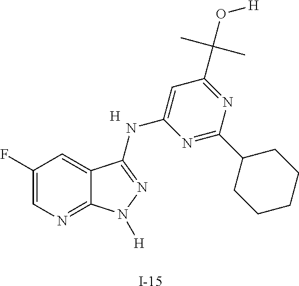

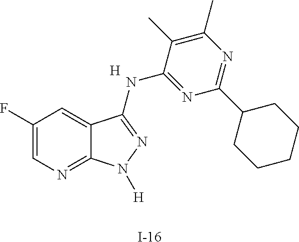

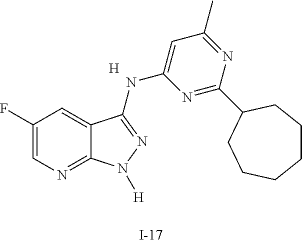

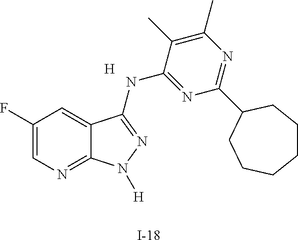

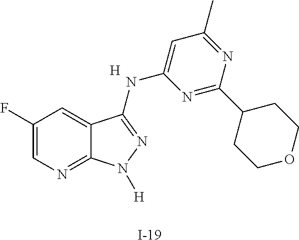

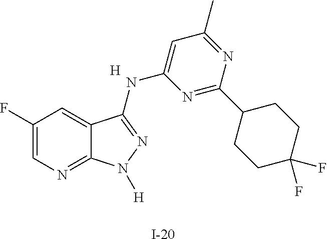

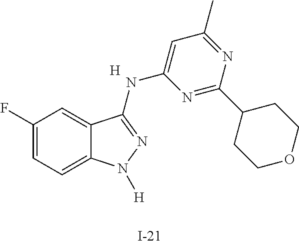

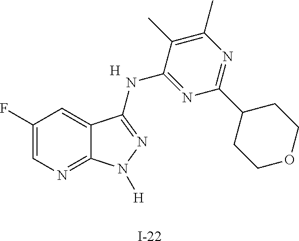

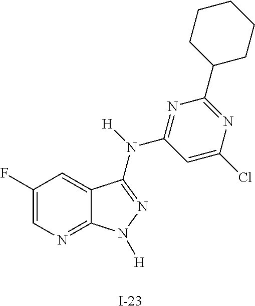

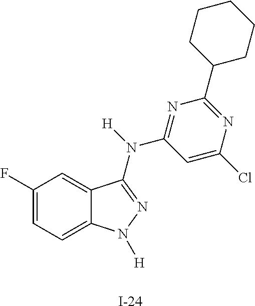

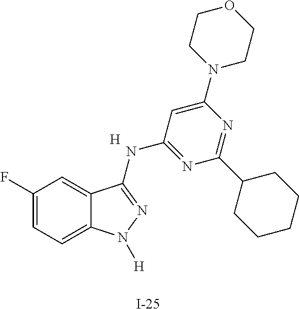

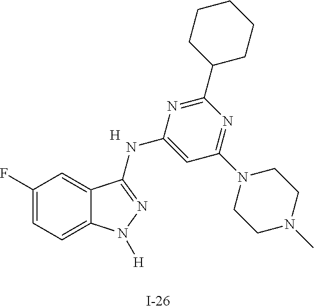

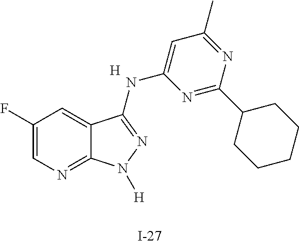

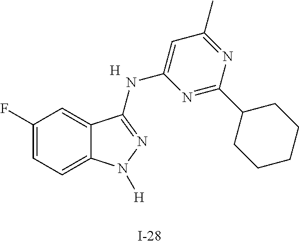

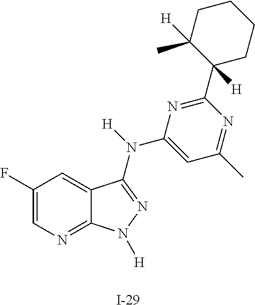

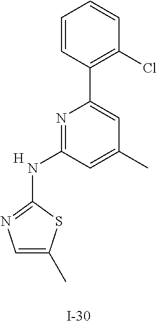

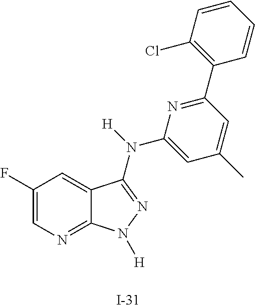

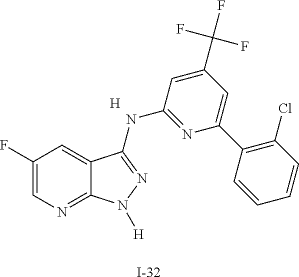

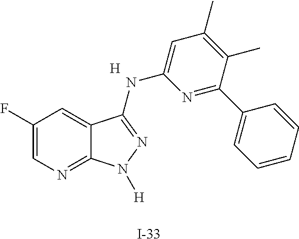

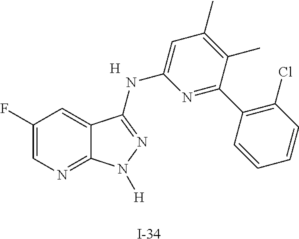

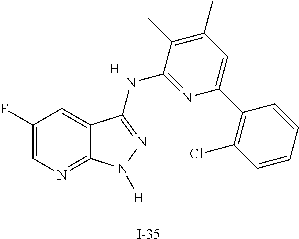

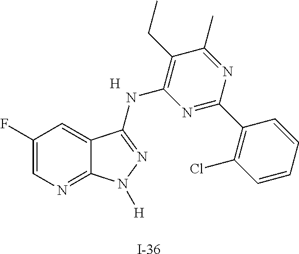

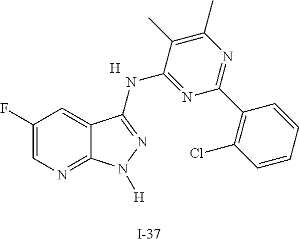

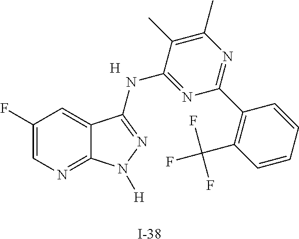

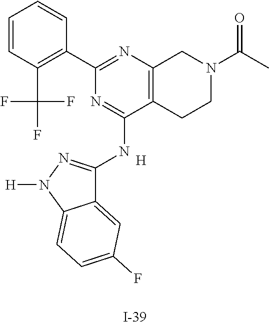

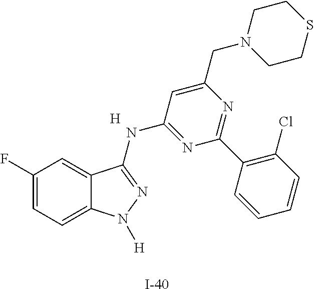

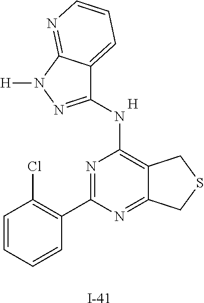

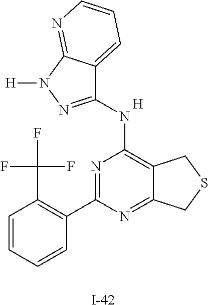

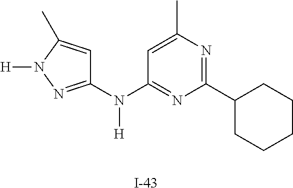

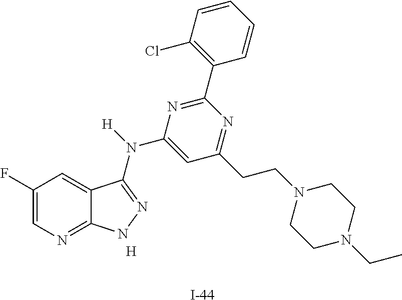

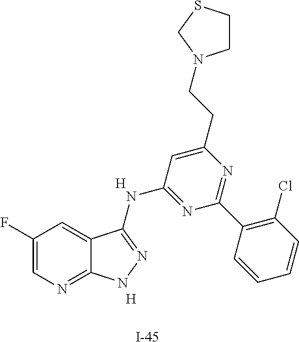

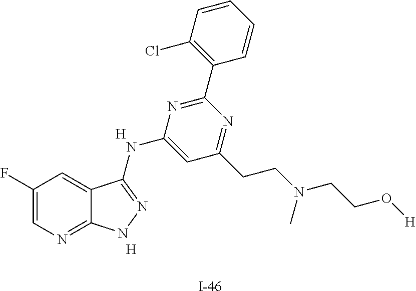

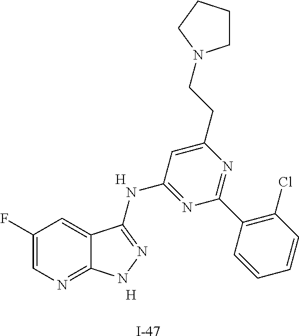

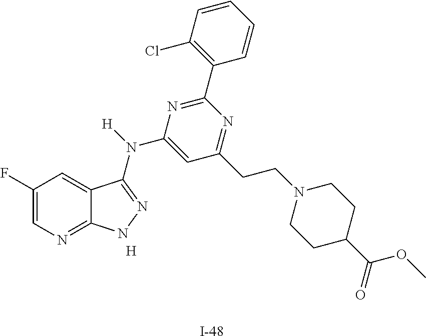

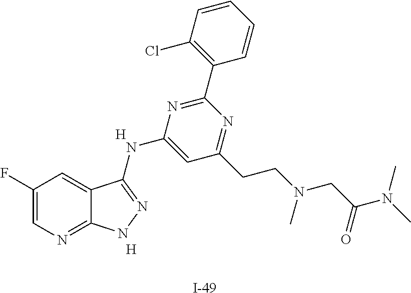

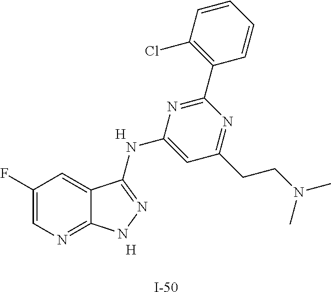

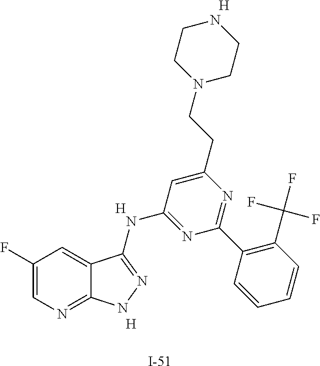

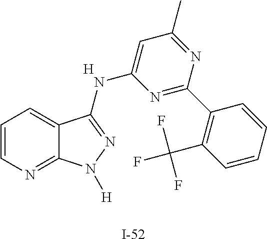

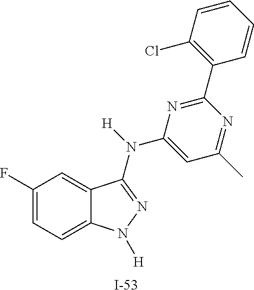

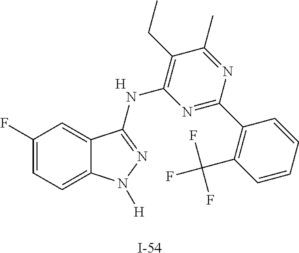

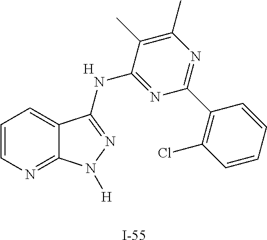

Synthetic Methods

The compounds of this invention may be prepared in general by methods such as those depicted in the general schemes below, and the preparative examples that follow. Unless otherwise indicated, all variables in the following schemes are as defined herein.

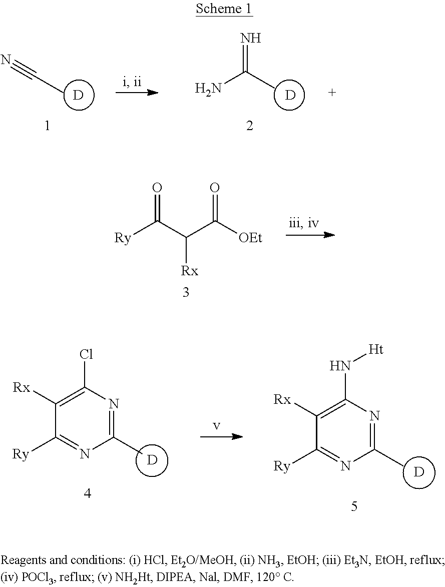

Scheme 1 above shows a general synthetic route that is used for preparing the compounds 5. Compounds of formula 5 can be prepared from intermediate 1. The formation of amidine 2 is achieved by treating nitrile derivative 1 with HCl in the presence of methanol and then treating the intermediate imidate with NH₃ in ethanol. Intermediate 2 is then treated with the corresponding beta-ketoester reflux in EtOH. The corresponding hydroxypyrimidine intermediate is treated with POCl₃ to yield chloroderivative 4. This reaction is amenable to a variety of amidines 2. The chloropyrimidine 4 is treated with diverse amines like NH₂Ht in the presence of DIPEA and NaI to yield the final compound 5. This reaction is also amenable to a variety of heterocyclic amines like NH₂Ht.

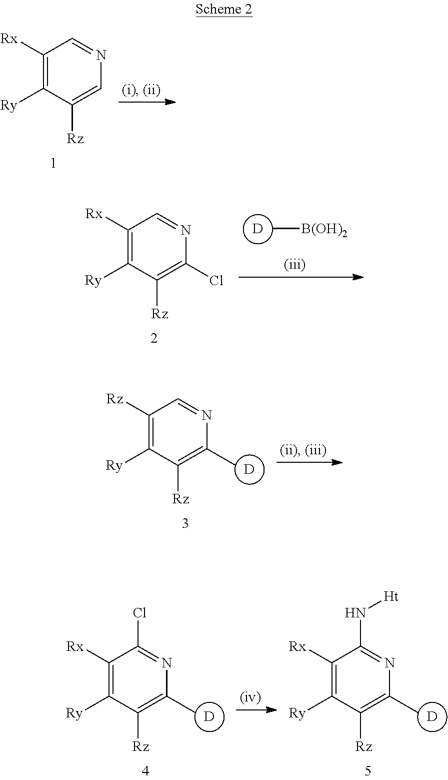

Scheme 2 above shows a general synthetic route that is used for preparing the compounds 5. Compounds of formula 5 can be prepared from intermediate 1. The formation of chloropyridine derivative 2 is achieved by treating the corresponding pyridine 1 with m-CPBA in EtOAc followed by conversion of the corresponding N-oxide to the chloropyridine by treating it with POCl₃. Intermediate 2 is then reacted with the corresponding boronic acid derivative to yield compound 3 using Suzuki coupling conditions well known for those skilled in the art. This reaction is amenable to a variety of boronic acid derivatives. The pyridine 3 is then converted in a chloropyridine derivative 4 using the same two step procedures as used in step l, m-CPBA oxidation followed by POCl₃ treatment. Intermediate 4 is then treated with diverse amines like NH₂Ht in the presence of Pd as a catalyst to yield the final compound 5. This reaction is also amenable to a variety of heterocyclic amines like NH₂Ht.

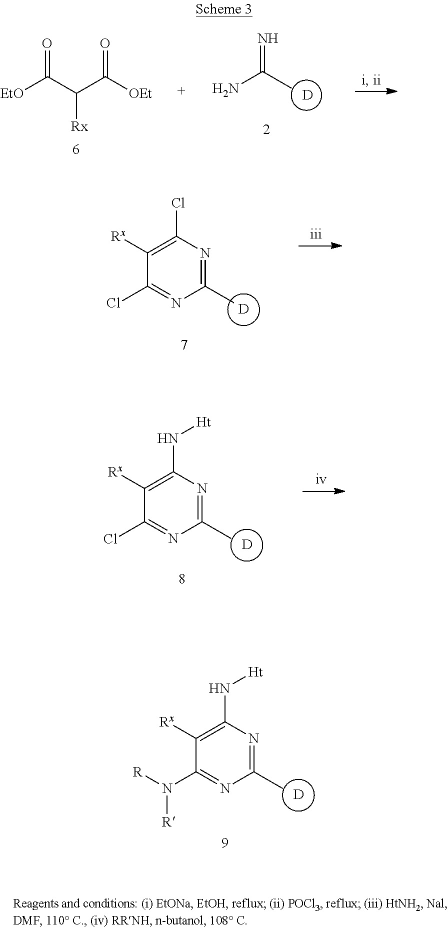

Scheme 3 above shows a general synthetic route that is used for preparing the compounds of formula 9. Compounds of formula 9 can be prepared from intermediate 7. The formation of intermediate 7 is achieved by reacting diethyl malonate 6 with the corresponding amidine 2 in the presence of EtONa as a base in refluxing ethanol. The crude is then treated with POCl₃ to yield dichloropyrimidine intermediate 7. The dichloropyrimidine intermediate is sequentially treated with heterocyclic amines and other substituted amine derivatives to yield final compounds 9. These two reactions sequence are amenable to a variety of heterocyclic amines and a variety of substituted amines.

In Scheme 3 above, R and R′ together with the nitrogen atom to which they are attached, form an optionally substituted 5-6 membered heterocyclic ring containing 1-2 heteroatoms selected from O, N, or S.

Therapeutic Uses

The present invention provides compounds and compositions that are useful as inhibitors of protein kinases.

As inhibitors of protein kinases, the compounds and compositions of this invention are particularly useful for treating or lessening the severity of a disease, condition or disorder where a protein kinase is implicated in the disease, condition, or disorder. In one aspect, the present invention provides a method for treating or lessening the severity of a disease, condition, or disorder where a protein kinase is implicated in the disease state. In another aspect, the present invention provides a method for treating or lessening the severity of a disease, condition, or disorder where inhibition of enzymatic activity is implicated in the treatment of the disease. In another aspect, this invention provides a method for treating or lessening the severity of a disease, condition, or disorder with compounds that inhibit enzymatic activity by binding to the protein kinase. Another aspect provides a method for treating or lessening the severity of a kinase disease, condition, or disorder by inhibiting enzymatic activity of the kinase with a protein kinase inhibitor.

As inhibitors of protein kinases, the compounds and compositions of this invention are also useful in biological samples. One aspect of the invention relates to inhibiting protein kinase activity in a biological sample, which method comprises contacting said biological sample with a compound of formula I or a composition comprising said compound. The term “biological sample”, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof. The term “biological sample” does not refer to in vivo samples.

Inhibition of protein kinase activity in a biological sample is useful for a variety of purposes that are known to one of skill in the art. Examples of such purposes include, but are not limited to, blood transfusion, organ-transplantation, and biological specimen storage.

Another aspect of this invention relates to the study of protein kinases in biological and pathological phenomena; the study of intracellular signal transduction pathways mediated by such protein kinases; and the comparative evaluation of new protein kinase inhibitors. Examples of such uses include, but are not limited to, biological assays such as enzyme assays and cell-based assays.

The activity of the compounds as protein kinase inhibitors may be assayed in vitro, in vivo or in a cell line. In vitro assays include assays that determine inhibition of either the kinase activity or ATPase activity of the activated kinase. Alternate in vitro assays quantitate the ability of the inhibitor to bind to the protein kinase and may be measured either by radiolabelling the inhibitor prior to binding, isolating the inhibitor/kinase complex and determining the amount of radiolabel bound, or by running a competition experiment where new inhibitors are incubated with the kinase bound to known radioligands.

Another aspect of this invention provides compounds that are chemomodulators of cellular differentiation.

In some embodiments, said protein kinase inhibitor is a GSK-3 inhibitor.

GSK-3

GSK-3 has been implicated in various diseases, disorders, and conditions including Diabetes, Alzheimer's, Huntington's and other neurodegenerative diseases, Amyotrophic Lateral Sclerosis, Parkinson's, Bipolar Disorder, Schizophrenia, Cerebral Stroke, Chemotherapeutic-dependent Leukocytopenia and Cardiac Hypertrophy. (PCT Publication Nos.: WO 99/65897 and WO 00/38675; Haq et al., J. Cell Biol. 2000, 151, pp. 117-130; Hirotani et al, Circulation Research 101, 2007, pp. 1164-1174). Inhibiting GSK-3 is the desired approach for treating these diseases, disorders, and conditions.

In cardiac hypertrophy, active GSK-3 may be important for inhibiting hypertrophy. However, blocking GSK-3 appears to be important for protecting against apoptosis in hypertrophied cardiac myoctyes. (Haq et al., J. Cell Biol. 2000, 151, pp. 117-130; Hirotani et al., Circulation Research 101, 2007, pp. 1164-1174).

GSK-3 regulates multiple downstream effectors associated with a variety of signaling pathways. These proteins include glycogen synthase, which is the rate limiting enzyme necessary for glycogen synthesis, the microtubule associated protein Tau, the gene transcription factor β-catenin, the translation initiation factor e1F2B, as well as ATP citrate lyase, axin, heat shock factor-1, c-Jun, c-myc, c-myb, CREB, and CEPBα. These diverse protein targets implicate GSK-3 in many aspects of cellular metabolism, proliferation, differentiation, and development.

In a GSK-3 mediated pathway that is relevant for the treatment of type II diabetes, insulin-induced signaling leads to cellular glucose uptake and glycogen synthesis. Along this pathway, GSK-3 is a negative regulator of the insulin-induced signal. Normally, the presence of insulin causes inhibition of GSK-3 mediated phosphorylation and deactivation of glycogen synthase. The inhibition of GSK-3 leads to increased glycogen synthesis and glucose uptake (Klein et al., PNAS 1996, 93, 8455-8459; Cross et al., Biochem. J. 1994, 303, pp. 21-26; Cohen, Biochem. Soc. Trans. 1993, 21, pp. 555-567; and Massillon et al., Biochem J. 1994, 299, pp. 123-128). In a diabetic patient, however, where the insulin response is impaired, glycogen synthesis and glucose uptake fail to increase despite the presence of relatively high blood levels of insulin. This leads to abnormally high blood levels of glucose with acute and long-term effects that may ultimately result in cardiovascular disease, renal failure and blindness. In such patients, the normal insulin-induced inhibition of GSK-3 fails to occur. It has also been reported that in patients with type II diabetes, GSK-3 is overexpressed. (See, PCT Application: WO 00/38675). Therapeutic inhibitors of GSK-3 are therefore potentially useful for treating diabetic patients suffering from an impaired response to insulin.

GSK-3 activity is associated with Alzheimer's disease. The hallmarks of this disease are the extracellular plaques formed by aggregated β amyloid peptides and the formation of intracellular neurofibrillary tangles via the tau protein.

It has been shown that GSK-3 inhibition reduces amyloid-β peptides in an animal model of Alzheimer's disease. (See, Phiel et. al., Nature 2003 423, 435-439, at pp. 435, 438). Mice over-expressing amyloid precursor protein (APP) treated with lithium (a GSK-3α inhibitor) over a three-week period showed over a 50% decrease in amyloid-β peptide tissue levels.

The neurofibrillary tangles contain hyperphosphorylated Tau protein, in which Tau is phosphorylated on abnormal sites. GSK-3 is known to phosphorylate these abnormal sites in cell and animal models. Conditional transgenic mice that over-express GSK-3 develop aspects of AD including tau hyperphosphorylation, neuronal apoptosis and spatial learning deficit. Turning off GSK-3 in these mice restores normal behavior, reduces Tau hyperphosphorylation and neuronal apoptosis. (Engel et al., J Neuro Sci, 2006, 26, pp. 5083-5090 and Lucas et al., EMBO J, 2001, 20, pp. 27-39). Inhibitors of GSK-3 have also been shown to prevent hyperphosphorylation of Tau in cells. (Lovestone et al., Current Biology 1994, 4, pp. 1077-86; and Brownlees et al., Neuroreport 1997, 8, pp. 3251-55).

GSK-3 also plays a role in psychosis and mood disorders, such as schizophrenia and bipolar disease. AKT haplotype deficiency, which correlated with increased GSK-3 activity, was identified in a subset of schizophrenic patients. A single allele knockout of GSK-30 resulted in attenuated hyperactivity in response to amphetamine in a behavior model of mania.

Several antipsychotic drugs and mood stabilizers used to treat both schizophrenic and bipolar patients have been shown to inhibit GSK-3 (Emamian et al., Nat Genet, 2004, 36, pp. 131-137; Obrien et al., J Neurosci, 2004, 24, pp. 6791-6798; Beaulieu et al., PNAS, 2004, 101, pp. 5099-5104; Li et al., Int J Neuropsychopharmacol, 2006, pp 1-13; Gould T D, Expert Opin Ther Targets, 2006, 10, pp. 377-392). Furthermore, a recent patent, US 2004/0039007, describes GSK 3 inhibitors that show mid-schizophrenic and anxiolytic effects in relevant mouse behavior models.

GSK-3 activity is also associated with stroke. Wang et al. showed that IGF-1 (insulin growth factor-1), a known GSK-3 inhibitor, reduced infarct size in rat brains after transient middle cerebral artery occlusion (MCAO), a model for stroke in rats. (Wang et al., Brain Res 2000, 859, pp. 381-5; Sasaki et al., Neurol Res 2001, 23, pp. 588-92; Hashimoto et al., J. Biol. Chem. 2002, 277, pp. 32985-32991). US 2004/0039007 describes the effect of GSK-3 inhibitors in MCAO, a stroke model in rats. These GSK-3 inhibitors significantly reduced striatal ischemic damage and reduced edema formation in rats. Additionally, the rats “demonstrated marked improvement in neurological function over the time course of the experiment.” US 2004/0039007, column 32, lines 47-50.

Inhibition of GSK-3 activity has been linked to stem cell proliferation, differentiation, neuronal plasticity and angiogenesis. These various functions are implicated in repair and regeneration. Inhibitors of GSK-3 have been shown to sustain self-renewal of embryonic stem cells, promote neuron, beta-cell, myeloid and osteoblast differentiation. (Sato et al., Nature Medicine 2004, 10, pp. 55-63; Ding et al., PNAS 2003, 100, pp. 7632-37; Branco et al., J Cell Science 2004, 117, pp. 5731-37; Trowbridge et al., Nature Medicine 2006, 12, pp. 89-98; Mussmann et al., JBC (Epub ahead of print) 2007; Kulkarni et al., Journal of Bone and Mineral Res. 2006, 21, pp. 910-920). With respect to neuronal plasticity, inhibition of GSK-3 has been shown to be important for regulating polarity, long-term potentiation (LTP) and neurite/axon growth. (Hooper et al., European J of Neuroscience 2007, 25, pp. 81-86; Kim et al., Neuron 2006, 52, pp. 981-996; Jiang et al., Cell 2005, 120, pp. 123-135). Inhibition of GSK-3 also has been shown to induce angiogenesis in endothelial cells. (Skurk et al., Circulation Research 2005, 96, pp. 308-318). Taken all together, GSK-3 small-molecule inhibitors have the potential to act as chemomodulators of repair and regeneration. This has implications for many types of degenerative conditions such as stroke, post-stroke recovery, Alzheimer's Disease, Parkinson's Disease, Huntington's Disease, Amyotrophic Lateral Sclerosis (ALS, Lou Gehrig's disease) Multiple Sclerosis (MS), Spinal Cord Injury, Traumatic Brain Injury, Charcot-Marie-Tooth, Leukocytopenia, Diabetes, Diabetic Neuropathy, Peripheral Nerve Regeneration, and Osteoporosis.

GSK-3 functions as both a tyrosine and a serine/threonine kinase, similar to the DYRK kinase family. Like the DYRK kinase family, GSK-3 auto-phosphorylates a key tyrosine residue in its kinase domain (GSK-3a, Tyr 279 and GSK-3b, Tyr 216). This tyrosine phosphorylation has been shown to be important for positively modulating kinase activity. Locheed et al, demonstrated that this autophosphorylation occurs intra-molecularly at a post-translationally intermediate step prior to maturation and is chaperone dependent (Lochhead et al, Molecular Cell 2006, 24, pp. 627-633). After maturation, GSK-3 loses its tyrosine kinase activity and acts exclusively as a serine and threonine kinase towards exogenous substrates.

β-catenin is one of the exogenous serine/threonine substrates that GSK-3 phosphorylates. Inhibition of β-catenin phosphorylation leads to an increase in β-catenin levels that, in turn, translocate to the nucleus and transcriptionally control many genes involved in cellular response and function. One potential safety concern for GSK-3 inhibitors is that use of the inhibitors could lead to hyperproliferation via β-catenin induction. As primarily a serine/threonine kinase, GSK-3 is central to many signalling pathways that control multiple cellular activities such as proliferation, differentiation and metabolism.

Because GSK-3 plays a central role in multiple signaling pathways, there is a therapeutic advantage when using compounds that can partially attenuate GSK-3 activity without completely blocking the enzyme and affecting multiple substrates such as β-catenin. In some embodiments, compounds that selectively inhibit the tyrosine autophosphorylation form of the enzyme over the serine/threonine kinase form provide those advantages for various GSK-3 associated disorders.

Surprisingly, compounds that selectively inhibit the auto-phosphorylation of the tyrosine form of the GSK-3 enzyme relative to the serine/threonine kinase form result in increased neuron growth and dendrite formation, such as increasing axonal and dendritic branching in neuronal cells. Similar results were observed with the production of new blood vessels from HUVECs. Increasing neuron growth and dendrite formation and angiogenesis are advantageous and provide improved therapeutic efficacy when treating many types of degenerative conditions such as Post-Stroke Recovery, Spinal Cord Injury, Traumatic Brain Injury, Alzheimer's disease, Parkinson's disease, Huntington's disease, Multiple Sclerosis, Amyotrophic Lateral Sclerosis, Diabetic Neuropathy, Charcot-Marie-Tooth, Leukocytopenia, Diabetes, Peripheral Nerve Regeneration, and Osteoporosis.

In another embodiment, the invention features a method of selectively inhibiting the auto-phosphorylation of the tyrosine form of the GSK-3 enzyme relative to the serine/threonine kinase form by administering a therapeutic effective amount of an inhibitor which selectively modulates the auto-phosphorylation of the tyrosine form of the GSK-3 enzyme relative to the serine/threonine kinase form. Accordingly, levels, amounts or dosages that selectively inhibit GSK-3α/β p-tyr or GSK-3 p-tyr are those levels, amounts or dosages that inhibit or modulate serine/threonine phosphorylation of the enzyme, relative to tyrosine auto-phosphorylation of the enzyme. The inhibitors can be used in vitro or in vivo.

In some embodiments, the inhibitor is a compound of formula I.

In some embodiments, the enzyme is GSK-3α; in other embodiments, the enzyme is GSK-3β.

In some embodiments, the method includes increasing axonal and dendritic branching in neuronal cells by administering a compound that selectively inhibits the auto-phosphorylation of the tyrosine form of the GSK-3 enzyme relative to the serine/threonine (kinase form. In some embodiments, the method includes promoting neuroplasticity by administering a compound that selectively inhibits the auto-phosphorylation of the tyrosine form of the GSK-3 enzyme relative to the serine/threonine kinase form. In other embodiments, the method includes promoting angiogenesis by administering a compound that selectively inhibits the auto-phosphorylation of the tyrosine form of the GSK-3 enzyme relative to the serine/threonine kinase form. In yet other embodiments, the method includes promoting neurogenesis by administering a compound that selectively inhibits the auto-phosphorylation of the tyrosine form of the GSK-3 enzyme relative to the serine/threonine kinase form. In yet other embodiments, methods include treating neuropsychiatric disorders, such as mania and depression, by administering a compound that selectively inhibits the auto-phosphorylation of the tyrosine form of the GSK-3 enzyme relative to the serine/threonine kinase form. In each of the foregoing embodiments, the compound can be a compound of formula I, such as one or more of compounds I-1 through I-55.

Another aspect of this invention provides a method for treating or lessening the severity of a disease, disorder, or condition selected from an autoimmune disease, an inflammatory disease, a proliferative or hyperproliferative disease, such as cancer, an immunologically-mediated disease, an immunodeficiency disorder, a bone disease, a metabolic disease, a neurological or neurodegenerative disease, a cardiovascular disease, allergies, diabetes, asthma, Alzheimer's disease, or a hormone-related disease, comprising administering an effective amount of a compound, or a pharmaceutically acceptable composition comprising a compound, to a subject in need thereof.

The term “cancer” includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal rectum; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma; Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma [embryonal rhabdomyosarcoma]), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis; Thyroid gland: papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions. In some embodiments, the cancer is selected from colorectal, thyroid, or breast cancer.

In some embodiments, said disease is chosen from allergic or type I hypersensitivity reactions, asthma, diabetes, Alzheimer's disease, Huntington's disease, Parkinson's disease, AIDS-associated dementia, bipolar disorder, amyotrophic lateral sclerosis (ALS, Lou Gehrig's disease), multiple sclerosis (MS), schizophrenia, leukocytopenia, cardiomyocyte hypertrophy, reperfusion/ischemia, stroke, baldness, transplant rejection, graft versus host disease, rheumatoid arthritis, and solid and hematologic malignancies. In some embodiments, said disease is chosen from diabetes, bipolar disorder, schizophrenia, stroke, Huntington's disease, leukocytopenia and cardiomyocyte hypertrophy. In some embodiments, said disease is a degenerative condition. In some embodiments, said degenerative condition is chosen from stroke, post-stroke recovery, Alzheimer's Disease, Parkinson's Disease, Huntington's Disease, Amyotrophic Lateral Sclerosis (ALS), multiple sclerosis (MS), spinal cord injury, traumatic brain injury, Charcot-Marie-Tooth, leukocytopenia, diabetes, diabetic neuropathy, peripheral nerve regeneration, and osteoporosis. In some embodiments, said disease is a neurodegenerative condition. In another embodiment, said neurodegenerative condition is selected from stroke, post-stroke recovery, Alzheimer's disease, Parkinson's disease, Huntington's disease, Amyotrophic Lateral Sclerosis (ALS), multiple sclerosis (MS), spinal cord injury, traumatic brain injury, peripheral nerve regeneration, other neurological disorders and Charcot-Marie-Tooth. As used herein, a neurological disorder is a disorder that affects the brain, spinal cord, nerves or muscles. As used herein, “post-stroke” includes post-stroke recovery, which includes treatment or improvement of the consequences of a stroke, the consequences including, including, without limitation, neuronal damage, behavioral changes, blood vessel damage and cell and tissue damage.

In other embodiments of this invention, said disease is a protein-kinase mediated condition. In some embodiments, said protein kinase in GSK-3.

The term “protein kinase-mediated condition”, as used herein means any disease or other deleterious condition in which a protein kinase plays a role. Such conditions include, without limitation, autoimmune diseases, inflammatory diseases, proliferative and hyperproliferative diseases, immunologically-mediated diseases, immuno-deficiency disorders, immunomodulatory or immunosuppressive disorder, bone diseases, metabolic diseases, neurological and neurodegenerative diseases, cardiovascular diseases, hormone related diseases, diabetes, allergies, asthma, and Alzheimer's disease.

The term “GSK-3-mediated condition”, as used herein means any disease or other deleterious condition in which GSK-3 plays a role. Such conditions include, without limitation, diabetes, diabetic neuropathy, osteoporosis, Alzheimer's disease, Huntington's disease, Parkinson's disease, AIDS-associated dementia, bipolar disorder, amyotrophic lateral sclerosis (ALS, Lou Gehrig's disease), multiple sclerosis (MS), schizophrenia, leukocytopenia, cardiomyocyte hypertrophy, stroke, post-stroke recovery, spinal cord injury, traumatic brain injury, Charcot-Marie-Tooth, peripheral nerve regeneration, and rheumatoid arthritis.

In some embodiments, the compounds are used to treat diabetes by promoting beta cell regeneration.

In other embodiments, the compounds are used to treat stroke patients during stroke recovery. In some cases, the compounds are used in post-stroke administration. The length of treatment can range from 1 month to one year. In some embodiments, the compound is administered after the stroke has occurred. In some embodiments, said administration during or right after ischemia. In some embodiments, the administration is conducted during or right after ischemia followed by continuous administration. For instance, the administration can begin during or after ischema and continue for 1 month to one year. In some embodiments, the administration begins within the 48 hours after ischema and continues for about 6 months. In any of the foregoing embodiments, the administration for providing post-stroke recovery can be given while the patient is undergoing physical therapy.

In yet other embodiments, the compounds are used to treat osteoporosis by osteoblastogenesis.

In still other embodiments, the invention features a method for identifying compounds useful for the treatment of GSK-3-mediated conditions by measuring the amount of auto-phosphorylation of the tyrosine form of the GSK-3 enzyme relative to the serine/threonine kinase form. In some aspects, amount of auto-phosphorylation of the tyrosine form of the GSK-3 enzyme relative to the serine/threonine kinase form is related to the ratio of β-catenin:GSK-3. In some embodiments, the method for identifying test compounds useful for the treatment of GSK-3-mediated conditions includes determining the ratio of β-catenin:GSK-3 by determining the β-catenin IC50 value for the test compound, determining the GSK-3α or GSK3β p-TYR IC50 value, and dividing the β-catenin IC50 value by the GSK-3α or GSK3β p-TYR IC50 value.

Another aspect of this invention provides a method of treating a GSK-3 mediated condition by administering a therapeutically effective amount of a compound, wherein the GSK-3 mediated condition is selected from diabetes, osteoporosis, Alzheimer's disease, Huntington's disease, Parkinson's disease, AIDS-associated dementia, bipolar disorder, amyotrophic lateral sclerosis, multiple sclerosis, schizophrenia, leukocytopenia, stroke, neurological disorders, peripheral nerve regeneration, and rheumatoid arthritis.

In some embodiments, the GSK-3 mediated condition is chosen from stroke, diabetes, schizophrenia, bipolar disortder, leukocytopenia, spinal cord injury, traumatic brain injury, Charcot-Marie-Tooth, and diabetic neuropathy.

In some embodiments, the GSK-3 mediated condition is stroke, and the compound may be administered after ischemia has occurred.

Another aspect of this invention provides a method of treating a GSK-3 mediated condition by administering a therapeutically effective amount of a compound, and also administering to said patient an additional therapeutic agent selected from an agent for treating diabetes, agent for treating osteoporosis, an agent for treating Alzheimer's disease, an agent for treating Huntington's disease, an agent for treating Parkinson's disease, an agent for treating AIDS-associated dementia, an agent for treating bipolar disorder, an agent for treating amyotrophic lateral sclerosis, an agent for treating multiple sclerosis, an agent for treating schizophrenia, an agent for treating leukocytopenia, an agent for treating peripheral nerve regeneration, an agent for treating stroke, and an agent for treating rheumatoid arthritis, wherein the additional therapeutic agent is appropriate for the disease being treated; and the additional therapeutic agent is administered together with said composition as a single dosage form or separately from said composition as part of a multiple dosage form.

In some embodiments, the additional therapeutic agent is selected from an agent for treating spinal cord injury, an agent for treating traumatic brain injury, an agent for treating Charcot-Marie-Tooth, or an agent for treating diabetic neuropathy.

Another aspect of this invention provides a method for treating a GSK-3 mediated condition comprising administering an agent that selectively inhibits the auto-phosphorylation of the tyrosine form of the GSK-3 enzyme relative to the serine/threonine kinase form.

In some embodiments, the agent comprises a compound of Formula I. In some embodiments, the GSK-3 mediated condition is Post-Stroke, Spinal Cord Injury, Traumatic Brain Injury, Alzheimers, Parkinsons, Huntington, Multiple Sclerosis, Amyotrophic Lateral Sclerosis, Diabetic Neuropathy, Charcot-Marie-Tooth, Leukocytopenia, Diabetes, Peripheral Nerve Regeneration, or Osteoporosis. In an embodiment, the GSK-3 mediated condition is Post-Stroke.

Yet another aspect of this invention provides a method of increasing axonal and dendritic branching in neuronal cells, comprising administering an agent that selectively inhibits the auto-phosphorylation of the tyrosine form of the GSK-3 enzyme relative to the serine/threonine kinase form.

In some embodiments, the agent that selectively inhibits the auto-phosphorylation of the tyrosine form of the GSK-3 enzyme relative to the serine/threonine kinase form is a compound of Formula I.

Another aspect of this invention provides a method of promoting neuroplasticity comprising, administering an agent that selectively inhibits the auto-phosphorylation of the tyrosine form of the GSK-3 enzyme relative to the serine/threonine kinase form.

In some embodiments, the agent is a compound of Formula I.

Another aspect of this invention provides a method of promoting angiogenesis comprising, administering an agent that selectively inhibits the auto-phosphorylation of the tyrosine form of the GSK-3 enzyme relative to the serine/threonine kinase form.

In some embodiments, the agent is a compound of Formula I.

Another aspect of this invention provides a method of promoting neurogenesis comprising administering an agent that selectively inhibits the auto-phosphorylation of the tyrosine form of the GSK-3 enzyme relative to the serine/threonine kinase form.

In some embodiments, the agent is a compound of Formula I.

Yet another aspect of this invention provides a method of treating neuropsychiatric disorders comprising administering an agent that selectively inhibits the auto-phosphorylation of the tyrosine form of the GSK-3 enzyme relative to the serine/threonine kinase form.

In some embodiments, the agent is a compound of Formula I. In some embodiments, the neuropsychiatric disorder is mania or depression.

Yet another aspect of this invention provides a method for identifying compounds useful for the treatment of GSK-3-mediated conditions comprising, measuring the amount of auto-phosphorylation of the tyrosine of the GSK-3 enzyme relative to the serine/threonine kinase form for one or more test compounds.

Another aspect of this invention provides a method for identifying compounds useful for the treatment of GSK-3-mediated conditions comprising, measuring the amount of auto-phosphorylation of the tyrosine of the GSK-3 enzyme and measuring the amount of phosphorylation of β-catenin.

In some embodiments, the step of measuring comprises obtaining the β-catenin IC50 value for the test compound, determining the GSK-3α or GSK3β p-TYR IC50 value, and dividing the β-catenin IC50 value by the GSK-3α or GSK3β p-TYR IC50 value.

Another aspect of this invention provides a method for identifying compounds useful for increasing axonal and dendritic branching in neuronal cells, comprising measuring the amount of auto-phosphorylation of the tyrosine form of the GSK-3 enzyme relative to the serine/threonine kinase form for one or more test compounds.

Yet another aspect of this invention provides a method for identifying compounds useful for increasing axonal and dendritic branching in neuronal cells, comprising measuring the amount of auto-phosphorylation of the tyrosine of the GSK-3 enzyme and measuring the amount of phosphorylation of β-catenin.

In some embodiments, the step of measuring comprises obtaining the β-catenin IC50 value for the test compound, determining the GSK-3α or GSK30 p-TYR IC50 value, and dividing the β-catenin IC50 value by the GSK-3α or GSK30 p-TYR IC50 value. In some embodiments, the method also comprises identifying compounds which exhibit a ratio of β-catenin IC50 to GSK-3α or GSK3β p-TYR IC50 of about 10 or higher. In some embodiments, the ratio is about 30 or higher.

Another aspect of this invention provides a method of providing post-stroke recovery, comprising administering an agent that selectively inhibits the auto-phosphorylation of the tyrosine form of the GSK-3 enzyme relative to the serineithreonine kinase form.

In some embodiments, the agent comprises a compound of Formula I. In some embodiments, the agent is administered during or immediately after ischemia. In some embodiments, the agent is administered during or immediately after ischemia and for a period of about 6 months after ischemia. In some embodiments, the method of providing post-stroke recovery further comprises administering physical therapy.

Another aspect of this invention provides a compound selected from compound I-39 through compound I-55.

Formulations and Routes of Administration

Another aspect provides pharmaceutically acceptable compositions comprising any of the compounds described herein and optionally comprising a pharmaceutically acceptable carrier, adjuvant or vehicle. In certain embodiments, these compositions optionally further comprise one or more additional therapeutic agents.

In certain embodiments, an “effective amount” of the compound or pharmaceutically acceptable composition is that amount effective in order to treat said disease. The compounds and compositions, according to the method of the present invention, may be administered using any amount and any route of administration effective for treating or lessening the severity of said disease. As used herein, “treating” or “treatment” includes preventing a disease, reducing the severity of a disease, or inhibiting a disease. As used herein, the term “prevents” refers to avoiding the condition, so that the condition does not occur in any way. The term “inhibits” refers to a reduction in the condition, or a slowing of the progress of the condition. The term “reduces” refers to a lessening of the condition or a slowing of the progress of the condition.

It will also be appreciated that certain of the compounds of present invention can exist in free form for treatment, or, where appropriate, as a pharmaceutically acceptable salt or pharmaceutically acceptable derivative thereof.

It should be understood that this invention includes mixtures/combinations of different pharmaceutically acceptable salts and also mixtures/combinations of compounds in free form and pharmaceutically acceptable salts.

As described herein, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents. Preservatives and antioxidants can also be present in the composition.

The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are another embodiment of the present invention. In some embodiments, said protein kinase-mediated condition is a GSK-3-mediated condition.

The exact amount of compound required for treatment will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal. In one embodiment, the animal is a mammal, and in another embodiment, the animal is a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed, including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well-known in the pharmaceutical formulating art. In such solid dosage forms, the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredients) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In addition to the compounds of this invention, pharmaceutically acceptable derivatives or prodrugs of the compounds of this invention may also be employed in compositions to treat or prevent the above-identified disorders.

A “pharmaceutically acceptable derivative or prodrug” means any pharmaceutically acceptable ester, salt of an ester or other derivative of a compound of this invention which, upon administration to a recipient, is capable of providing, either directly or indirectly, a compound of this invention or an inhibitorily active metabolite or residue thereof. Particularly favoured derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Pharmaceutically acceptable prodrugs of the compounds of this invention include, without limitation, esters, amino acid esters, phosphate esters, metal salts and sulfonate esters.

Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, infrasternal, intrathecal, intrahepatic, intralesional and intracranial injection or infusion techniques. In certain embodiments, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of protein kinase inhibitor that may be combined with the carrier materials to produce a single dosage form will vary depending upon the host treated, and the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, and diet of the patient, time of administration, rate of excretion, drug combination, the judgment of the treating physician and the severity of the particular disease being treated. The amount of inhibitor will also depend upon the particular compound in the composition.

Depending upon the particular protein kinase-mediated conditions to be treated or prevented, additional drugs, which are normally administered to treat or prevent that condition, may be administered together with the inhibitors of this invention. For example, chemotherapeutic agents or other anti-proliferative agents may be combined with the protein kinase inhibitors of this invention to treat proliferative diseases.

Those additional agents may be administered separately, as part of a multiple dosage regimen, from the protein kinase inhibitor-containing compound or composition. Alternatively, those agents may be part of a single dosage form, mixed together with the protein kinase inhibitor in a single composition.

In some embodiments, said protein kinase inhibitor is a GSK-3 kinase inhibitor.

This invention may also be used in methods other than those involving administration to a patient.

### EXAMPLES

The compounds of this invention may be prepared in general by methods known to those skilled in the art. Those compounds may be analyzed by known methods, including but not limited to LCMS (liquid chromatography mass spectrometry) and NMR (nuclear magnetic resonance). Compounds of this invention may be also tested according to these examples. It should be understood that the specific conditions shown below are only examples, and are not meant to limit the scope of the conditions that can be used for making, analyzing, or testing the compounds of this invention. Instead, this invention also includes conditions known to those skilled in that art for making, analyzing, and testing the compounds of this invention.

As used herein, the term “Rt(min)” refers to either HPLC (high performance liquid chromatography) or LCMS retention time, in minutes, associated with the compound.

Unless otherwise indicated, the HPLC method utilized to obtain the reported retention time is as follows:

Column: ACE C8 column, 4.6×150 mm

Gradient: 0-100% acetonitrile+methanol 60:40 (20 mM Tris phosphate)

Flow rate: 1.5 mL/minute

Detection: 225 nm.

LCMS (Liquid Chromatography Mass Spectrometry) samples were analyzed on a MicroMass Quattro Micro mass spectrometer operated in single MS mode with electrospray ionization. Samples were introduced into the mass spectrometer using chromatography. Mobile phase for all mass spectrum analysis consisted of acetonitrile-water mixtures with either 0.2% formic acid or 0.1% TFA as a modifier. Column gradient conditions are 10%-90% acetonitrile over 3 minutes gradient time and 5 minutes run time on a Waters YMC Pro-C18 4.6×50 mm column. Flow rate was 1.5 ml/min.

¹H-NMR spectra were recorded at 400 MHz using a Bruker DPX 400 instrument. The following compounds of formula I were prepared and analyzed as follows.

### Example 1

### Intermediates

### Intermediate 1

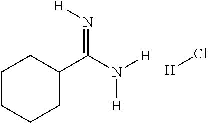

### Cyclohexanecarboximidamide Hydrochloride

The temperature of a mixture of cyclohexane carbonitrile (60 g, 550 mmol, 1 eq) in Et₂O (150 ml) and MeOH (33 ml) was lowered to 0° C. before HCl(g) was bubbled through for 20 minutes. The reaction mixture was then removed to the fridge O/N. The resulting white solid was suspended in Et₂O and filtered to give the methyl cyclohexanecarbimidate intermediate (125.1 g, 128%). This crude solid was suspended in a mixture of EtOH (400 ml)/2M NH₃ in EtOH (100 ml) at 0° C. before NH₃(g) was bubbled through the suspension for 2 hours. The reaction mixture was then placed in the fridge overnight. The resulting solid was filtered and washed with MeOH to yield a filtrate which was then concentrated in-vacuo. The residue was taken up in MeOH and concentrated in-vacuo until a solid began to precipitate, at which time Et₂O was added. The resulting solid that formed was filtered to give a sticky solid that was placed into the vac-oven overnight to yield the desired product as a white solid (87.80 g, 98%). 1H (400 MHz, DMSO) 1.00-1.88 (10H, m), 2.35-2.57 (1H, m), 8.86-9.02 (3H, m).

### Intermediate 2

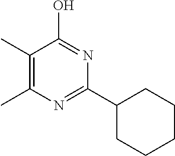

### 2-cyclohexyl-5,6-dimethylpyrimidin-4-ol

A solution of sodium ethoxide (previously prepared by dissolving sodium (6.36 g, 278 mmol, 3 eq) in EtOH (300 ml)), stirring at room temperature, was treated with ethyl 2-methyl-3-oxobutanoate (16.94 ml, 120 mmol, 1.3 eq). A slurry of cyclohexanecarboxitnidamide hydrochloride (15 g, 92 mmol, 1 eq) in EtOH (100 ml) was then added and the resulting mixture heated at reflux for 8 hours. The resulting mixture was concentrated, water was added, and the pH was adjusted to ˜7-8 with 2N HCl. Following acidification, a white solid precipitated and this was filtered and dried in the vac-oven to yield 2-cyclohexyl-5,6-dimethylpyrimidin-4-ol as a white solid (28.9 μg, 151%). 1H (400 MHz, DMSO) 1.03-1.86 (10H, m), 1.96 (3H, s), 2.16 (3H, s), 2.36-2.57 (1H, m), 12.05 (1H, brs); ES+207.

### Intermediate 3

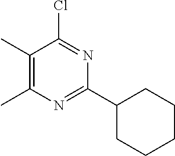

### 4-chloro-2-cyclohexyl-5,6-dimethylpyrimidine

POCl₃ (220 ml, 2.4 mol, ˜26 eq) was cooled to ˜−50° C. before being carefully treated with 2-cyclohexyl-5,6-dimethylpyrimidin-4-ol (28.9 g, ‘92 mmol’, 1 eq). The cooling bath was then removed and the pot allowed to warm to room temperature, followed by heating to reflux for 6 hours. The resulting mixture was concentrated, treated with ice and saturated NaHCO₃ and extracted into Et₂O before being dried (sodium sulfate)/concentrated. The resulting oil was subjected to column chromatography using EtOAc (10%): 40-60 Petrols (90%) as eluent to yield 4-chloro-2-cyclohexyl-5,6-dimethylpyrimidine as an oil (5.7011 g, 28% over first two steps). 1H (400 MHz, DMSO) 1.16-1.90 (10H, m), 2.26 (3H, s), 2.46 (3H, s), 2.60-2.75 (1H, m). ES+225.

### Intermediate 4

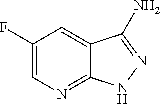

The overall synthetic scheme for the synthesis of 5-fluoro-1H-pyrazolo[3,4-b]pyridin-3-amine 5 is depicted below.

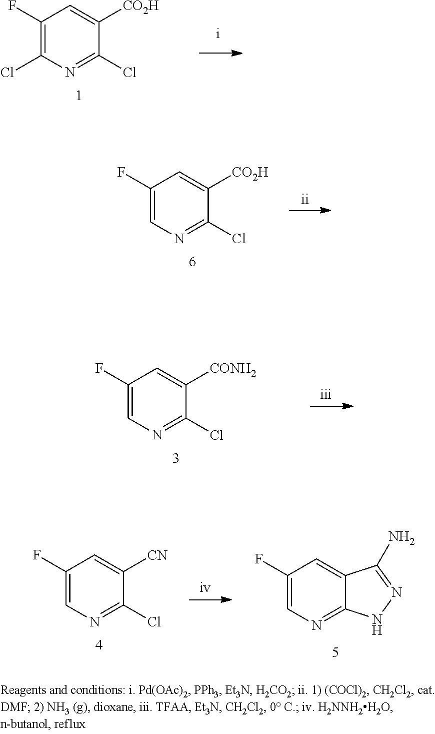

### 2-Chloro-5-fluoronicotinic acid (6)

To a round-bottomed flask under a N₂ atmosphere were added degassed DMF (270 mL), Pd(OAc)₂ (0.05 eq, 2.7 g, 11.9 mmol), PPh₃ (0.1 eq, 6.2 g, 23.8 mmol) and degassed Et₃N (6 eq, 200 mL, 1428.6 mmol). The mixture was stirred 20 minutes, then HCOOH (3 eq, 28 mL, 714.3 mmol) was added, followed after 5 minutes by 2,6-dichloro-5-fluoronicotinic acid (50 g, 238.1 mmol), and the mixture was stirred at 50° C. The reaction was followed by analysis (1H NMR) of a worked-up aliquot. When all starting material was consumed (24 hours), the mixture was cooled to 0° C. and water (500 mL) was added. After 20 minutes, the mixture was filtered through a pad of Celite that was rinsed with water. The mixture was basified to pH 9 with 30% aqueous NaOH and washed with EtOAc (2×). HCl (12 N) was added slowly to pH 1 and the solution was saturated with NaCl. The mixture was extracted with EtOAc (3×). The combined organic extracts were washed with brine, dried (Na₂SO₄) and concentrated under reduced pressure to give 37 g (88%) of a beige solid used in the next step without further purification. ¹H NMR (DMSO-d₆, 300 MHz): δ 8.16 (dd, 1H); 8.58 (d, 1H).

### 2-Chloro-5-fluoronicotinamide (3)

To a solution of 2-chloro-5-fluoronicotinic acid 6 (50 g, 285 mmol) and DMF (2 mL, 28 mmol) in DCM (400 mL) at 0° C. was added oxalyl chloride (64 mL, 741 mmol) dropwise. The reaction mixture was stirred at room temperature overnight and concentrated in-vacuo. The resulting yellow liquid was dissolved in 1,4-dioxane (600 mL), cooled at 0° C., and NH₃(g) was bubbled through the solution for 30 minutes. The mixture was stirred at room temperature overnight. The resulting mixture was filtered and the filtrate was concentrated to give compound 3 (44 g, 89%) as a beige solid. NMR (DMSO-d₆, 300 MHz): δ 7.84 (s, 1H), 7.96 (dd, 1H), 8.09 (s, 1H), 8.49 (d, 1H).

### 2-Chloro-5-fluoronicotinonitrile (4)

A suspension of crude compound 3 (65 g, 372.4 mmol) and Et₃N (114 mL, 819.2 mmol) in DCM (700 mL) was cooled to 0° C. and TFAA (57 mL, 409.6 mmol) was added dropwise. The resulting yellow solution was stirred for 90 minutes at 0° C., diluted with DCM, washed with saturated aqueous NaHCO₃ and brine, and dried (Na₂SO₄). The mixture was filtered and concentrated. Kugel Rohr distillation of the residue (−70° C./1 mbar) gave 50 g (86%) of compound 4 as a beige solid.

Compound 4 can also be purified by column chromatography (SiO₂, 8:1 heptane:EtOAc). ¹H NMR (CDCl₃, 300 MHz): δ 7.78 (dd, 1H); 8.49 (d, 1H).

### 5-Fluoro-1H-pyrazolo[3,4-b]pyridin-3-amine (5)

To a solution of compound 4 (50 g, 321.7 mmol) in 1-butanol (1 L) was added hydrazine monohydrate (150 mL, 3.2 mol), and the mixture was refluxed for 4 hours. The mixture was cooled to room temperature and concentrated. The precipitate was successively washed on filter with water (2×) and Et₂O (2×) and dried in-vacuo overnight to give compound 5 (44 g, 88%) as a yellow solid. NMR (DMSO-d₆, 300 MHz): δ 5.53 (s, 2H); 7.94 (dd, 1H); 8.35 (dd, 1H); 12.02 (s, 1H).

### Intermediate 5

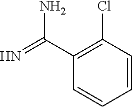

### 2-chlorobenzimidamide

2-Chlorobenzonitrile (26.84 g, 195 mmol) was added in 6 portions over 25 minutes to a stirred solution of LHMDS (1M in THF, 400 mL, 400 mmol) in ether (400 mL) with ice-bath cooling under nitrogen. After 5 minutes, the cooling bath was removed and the stirring continued overnight. LCMS shows the reaction now complete (around 50% complete after 3.5 hours). Aqueous HCl (3M, 400 mL) was added carefully with ice-bath cooling followed by ether (600 mL) and water (600 mL) and extraction was carried out. The organic layer was re-extracted with aqueous HCl (400 mL). The combined aqueous layers were basified with solid NaOH carefully to pH 14 and then extracted with DCM (×3), dried (K₂CO₃), filtered and concentrated in-vacuo to give the amidine as a white solid (26.93 g, 89.3%).

1H NMR (DMSO) 6.34 (3H, s), 7.32-7.40 (3H, m), 7.46 (1H, dd).

### Intermediate 7

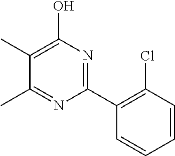

### 2-(2-chlorophenyl)-5,6-dimethylpyrimidin-4-ol

To 2-chlorobenzimidamide (34.07 g, 220 mmol) and triethylamine (44.50 g, 440 mmol) in ethanol (750 mL) was added ethyl 2-methyl-3-oxobutanoate (38.13 g, 264 mmol) and heated at 90° C. for 4 hours. A further portion (6.36 g) of the ester was added and stirred for 3 hours. The reaction was concentrated to around 500 mL and stood overnight. Precipitation of the desired pyrimidinol (22.7 g) occurred. The mother liquors were concentrated and DCM and 1M HCl were added. The aqueous layer was extracted seven times with DCM to give further crops of the desired product (total: 28.9 g, 56%) on concentration as a white solid.

1H NMR (DMSO) 1.97 (3H, s), 2.26 (3H, s), 7.44-7.47 (1H, m), 7.51-7.59 (3H, m), 12.70 (1H, br s).

### Intermediate 8

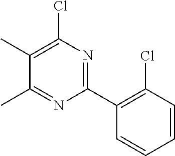

### 4-chloro-2-(2-chlorophenyl)-5,6-dimethylpyrimidine

The following reaction was split into two parts and carried out simultaneously in two identical reaction vessels:

To the pyrimidinol (14.45 g, 61.6 mmol) was added POCl₃ (50 mL) carefully and stirred for 5 minutes. Then a further portion of POCl₃ (100 mL) was added and heated to 105° C. After 1 hour at this temperature, the reaction was concentrated. Ice was added and the reactions, run in duplicate, were combined with the aid of DCM. The organic layer was washed with brine and water and dried (MgSO₄), filtered and concentrated in-vacuo to give the chloropyrimidine (31.14 g, 99.8%) as a colourless oil.

1H NMR (CDCl₃) 2.45 (3H, s), 2.65 (3H, s), 7.36-7.39 (2H, m), 7.49-7.51 (1H, m), 7.71-7.73 (1H, m).

### Example 2

### Compound I-37

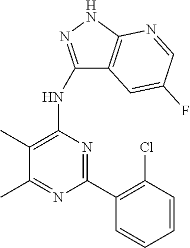

### N-(2-(2-chlorophenyl)-5,6-dimethylpyrhnidin-4-yl)-5-fluoro-1H-pyrazolo[3,4-b]pyridin-3-amine

A mixture of 4-chloro-2-(2-chlorophenyl)-5,6-dimethylpyrimidine (31.14 g, 123 mmol) and 5-fluoro-1H-pyrazolo[3,4-b]pyridin-3-amine (19.65 g, 129 mmol) in NMP (200 mL) was heated at 135° C. for 3 hours and 30 minutes. Then the mixture was concentrated in-vacuo to around 100 mL. Then saturated aqueous NaHCO₃, water and EtOAc were added, and a precipitate appeared in the organic layer. The whole mixture was filtered off and the residue was washed with saturated aqueous NaHCO₃, water, EtOAc and ether. Boiling ethanol was added to the residue with stirring and the pure target compound was filtered. The liquors were concentrated and this trituration procedure was repeated four times to give the target (25 g, 55%) as a white solid.

1H NMR (DMSO) 2.28 (3H, s), 2.43 (3H, s), 7.28-7.37 (2H, m), 7.40-7.46 (2H, m), 7.93 (1H, dd), 8.49 (1H, s), 9.28 (1H, br s), 13.39 (1H, br s).

### Example 3

### Compound I-38

N-(5,6-dimethyl-2-(2-(trifluoromethyl)phenyl)pyrimidin-4-yl)-5-fluoro-1H-pyrazolo[3,4-b]pyridin-3-amine is prepared according to Scheme I as shown below:

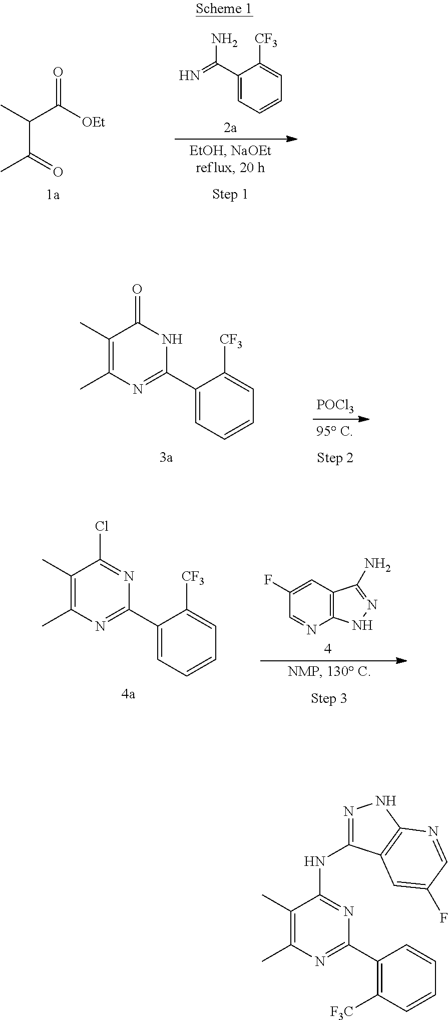

### Intermediate 3a

### 5,6-dimethyl-2-(2-(trifluoromethyl)phenyl)pyrimidin-4(3H)-one

A solution of the beta-ketoester 1 (7.5 mL, 58 mmol) dissolved in ethanol (250 mL) is prepared. After cooling to 0° C. with an external ice bath, amidine hydrochloride 2 (63.8 mmol) and sodium ethoxide (15.8 g, 232 mmols (4 equiv)) are added in portions to the solution. The temperature of the reaction is kept at 0° C. during the addition. The reaction mixture is then refluxed for 20 hours, and then checked for completion by HPLC/TLC (thin-layer chromatography) (6.25% EtOAc-Hexane). Upon completion, the solvent is removed, and the residue is taken up with a mixture of brine and EtOAc. The reaction is extracted several times with EtOAc. (NOTE: multiple extractions may be necessary to obtain all the material from the aqueous portion.) The combined organic extracts are dried with sodium sulfate and filtered. The solvent is evaporated to give the crude product, which is purified by placing the material on a plug of silica gel and eluting it with 5-25% EtOAc/Hexane to give intermediate 3a.

### Intermediate 4a

### 4-chloro-5,6-dimethyl-2-(2-(trifluoromethyl)phenyl)pyrimidine

The lactam 3 (58 mmol) is treated with POCl₃ neat (25-50 mL), and heated at reflux for several hours until all the starting material is consumed (as monitored and determined based on HPLC/TLC). The solvent is removed under reduced pressure, and then the product is quenched with ice and brine. The product is extracted using EtOAc until no product appears (monitored and determined based on TLC. NOTE: multiple extractions may be necessary to obtain all the material from the aqueous layer). Filtration and removal of the solvent gives the crude chloropyrimidine, which is purified by placing the material on a plug of silica gel and eluting with 0-10% EtOAc/Hexane to give intermediate 4.

### Compound I-38

### N-(5,6-dimethyl-2-(2-(trifluoromethyl)phenyl)pyrimidin-4-yl)-5-fluoro-1H-pyrazolo[3,4-b]pyridin-3-amine

The chloropyrimidine 4a (10.0 mmol) is dissolved in NMP (5-10 mL), followed by addition of the amine 4 (1.5 g, 11 mmol). The reaction mixture is refluxed for ˜4 hours, and then checked for completion by HPLC/TLC (6.25% EtOAC-Hexane). The completed reaction is diluted with brine, and extracted with EtOAc several times. After drying over Na₂SO₄, the product is filtered and reduced under vacuum to give the crude product. Purification elution through a plug of silica gel (10-75% EtOAc-Hexane) is carried out. The homogeneous fractions are combined and stripped to give the free base. The HCl salt is then prepared by dissolving the product with MeOH and adding excess HCl in dioxane followed by removal of the solvent under vacuum. The final product is obtained by triturating the resultant glass with EtOAc to give the salt as a solid. The salt is then dried under high vacuum at 100° C. to remove all traces of solvent to provide a 33% yield of the title compound over 3 steps. LCMS rt(min)=2.40; MH₊=403.1.

### Example 4

### Compound I-16

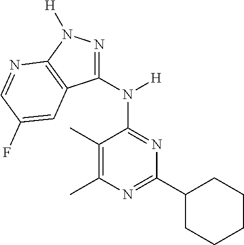

### (N-(2-cyclohexyl-5,6-dimethylpyrimiidin-4-yl)-5-fluoro-1H-pyrazolo[3,4-b]pyridin-3-amine

A solution of 4-chloro-2-cyclohexyl-5,6-dimethylpyrimidine (5.70 g, 25.4 mmol, 1 eq) in NMP (50 ml), stirring at room temperature, was treated with 5-fluoro-1H-pyrazolo[3,4-b]pyridin-3-amine (4.63 g, 30.4 mmol, 1.2 eq). The resulting mixture was heated at 130° C. for 4 hours before being cooled to room temperature. The resulting mixture was diluted with EtOAc/water and the organics were washed with sNaHCO₃ and water. During work up, a solid was produced and this was filtered. Treatment of the solid with DCM/MeOH/40-60-petrols produced (N-(2-cyclohexyl-5,6-dimethylpyrimiidin-4-yl)-5-fluoro-1H-pyrazolo[3,4-b]pyridin-3-amine, which was isolated as a white solid. The solid was dried in a vac-oven @ 80° C. overnight to yield VRT-763633 (Lot 2) as a white solid (5.2608 g, 61%)). 1H (400 MHz, DMSO) 0.87-1.22 (5H, m), 1.40-1.62 (5H, m), 2.04 (3H, s), 2.20 (3H, s), 2.25 (1H, quin), 7.67 (1H, dd), 8.40 (1H, dd), 8.91 (1H, s), 13.13 (1H, s). ES+341, ES-339.

### Example 5

### Compound I-32

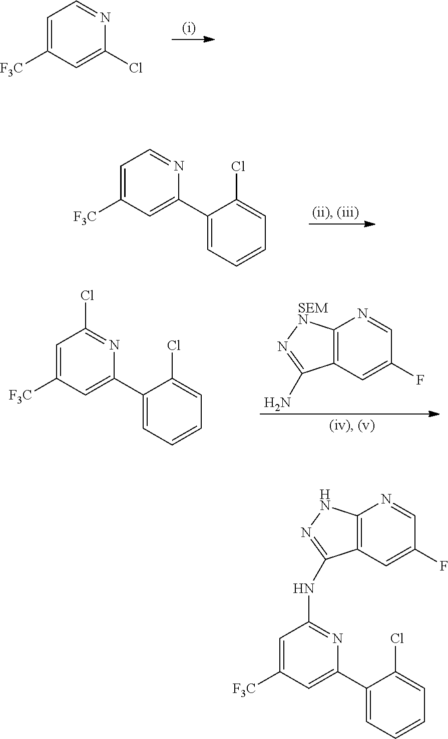

### Step (i)

### 2-(2-chlorophenyl)-4-(trifluoromethyl)pyridine

A mixture of 2-chloro-4-(trifluoromethyl)pyridine (4 g, 22.0 mmol), 2-chlorophenylboronic acid (5.04 g, 24.2 mmol), Ba(OH)₂ (12.5 g, 66.1 mmol) and Pd(PPh₃)₂Cl₂ (464 mg, 0.66 mmol) in DME (140 mL)/water (35 mL) was heated at 110° C. for 1 hour. The solids were filtered off and the mother liquors were concentrated to around 80 mL and extracted with EtOAc. The organic layer was washed with brine then water, dried (MgSO₄), filtered and concentrated. Purification by column chromatography (1/1 Petroleum ether/EtOAc) gave the Suzuki adduct (5.05 g, 89%) as a colourless oil.

¹H NMR (400 MHz, CDCl₃): 7.41-7.44 (2H, m), 7.51-7.56 (2H, m), 7.63-7.65 (1H, m), 7.94 (1H, s), 8.94 (1H, d).

### Step (ii), (iii)

### 2-chloro-6-(2-chlorophenyl)-4-(trifluoromethyl)pyridine

To a solution of 2-(2-chlorophenyl)-4-(trifluoromethyl)pyridine (5.40 g, 21.0 mmol) in EtOAc (100 mL) was added mCPBA (7.25 g, 42.0 mmol) in EtOAc (100 mL) over 30 min and then the mixture was heated at reflux (caution: use shield) for 3 hours. The reaction mixture was cooled and then washed twice with saturated aqueous NaHCO₃ and concentrated.

The residue from above was dissolved in POCl₃ (50 mL) and refluxed for 45 minutes. The mixture was concentrated and then ice was added. After 1 hour, the mixture was extracted with EtOAc and then the organic layer was washed with brine, dried (MgSO₄), filtered and concentrated. Purification by column chromatography (10/1 Petroleum ether/EtOAc) gave the chloropyridine (4.48 g, 73%) as a colourless oil.

¹H NMR (400 MHz, CDCl₃): 7.41-7.45 (2H, m), 7.50-7.55 (1H, m), 7.59 (1H, s), 7.65-7.70 (1H, m), 7.88 (1H, s).

### Step (iv), (v)

### N-(6-(2-chlorophenyl)-4-(trifluoromethyl)pyridin-2-yl)-5-fluoro-1H-pyrazolo[3,4-b]pyridin-3-amine

Xantphos (37.7 mg, 0.065 mmol) was added to Pd(OAc)₂ (7.3 mg, 0.033 mmol) in dioxane (2 mL). After 3 minutes, this solution was added (with the aid of 1 mL dioxane) to a mixture of 2-chloro-6-(2-chlorophenyl)-4-(trifluoromethyl)pyridine (85.4 mg, 0.359 mmol), 5-fluoro-1-((2-(trimethylsilyl)ethoxy)methyl)-1H-pyrazolo[3,4-b]pyridin-3-amine (92 mg, 0.326 mmol) and potassium carbonate (225 mg, 1.63 mmol) in dioxane (15 mL) under nitrogen. The reaction mixture was heated and stirred at reflux for 80 minutes. EtOAc was added, the solids were filtered off and the filtrate was concentrated.

Purification by column chromatography (1/2 Petroleum ether/EtOAc) gave a residue that was dissolved in THF (2 mL) and treated with aqueous HCl (2 M, 8 mL). After 90 minutes at 100° C., the mixture was concentrated. The residue was partitioned between EtOAc and aqueous saturated NaHCO₃. The organic layer was separated, dried (MgSO₄), filtered and concentrated. The residue was purified by Fractionlynx mass-directed preparation. HPLC (with aqueous TFA/MeCN mixtures) and the fractions passed through a bicarbonate Stratosphere cartridge. Concentration gave the aminopyridine (15 mg, 11%) as a white solid. ¹H NMR (400 MHz, DMSO): 7.36 (1H, s), 7.48-7.51 (2H, m), 7.60-7.64 (2H, m), 8.31 (1H, s), 8.43 (1H, d), 8.56 (1H, s), 10.61 (1H, s), 13.17 (1H, s).

### Example 6

### Physical Data

The compounds were synthesized using the methods described herein as well as those known in the art. Physical data for compounds I-1 through I-55 is provided in Table 2.

TABLE 2

| Patent | LCMS | LCMS | |
|---|---|---|---|
| Cmpd # | (M + 1) | Rt (min) | HNMR |
| I-1 | 380.1 | — | 1HNMR (500 MHz, DMSO-d6) δ2.18 (m, 2H), 2.89 |
| | | | (m, 2H), 3.02 (t, 2H), 7.24 (td, 1H), 7.42 (m, 2H), |
| | | | 7.49 (td, 1H), 7.52 (dd, 1H), 7.54 (d, 1H), 7.57 (dd, |
| | | | 1H), 10.50 (br. s, 1H), 13.06 (br. s, 1H) |
| I-2 | 402.2 | — | 1HNMR (500 MHz, DMSO) δ 2.27 (3H, s), 2.40 |
| | | | (3H, s), 7.16 (2H, m), 7.44 (2H, m), 7.52 (1H, J = 7.4 |
| | | | Hz, t), 7.57 (1H, J = 7.4 Hz, t), 7.67 (1H, J = 7.8 Hz, |
| | | | d), 9.03 (1H, s), 12.75 (1H, s) ppm; |
| I-3 | 368.3 | 1.9 | H NMR (500 MHz, DMSO) 13.2 (s, 1H), 10.55 (s, |
| | | | 1H), 7.59-7.51 (m, 3H), 7.45-7.39 (m, 2H), 7.24 |
| | | | (td, J = 9.1, 3.8 Hz, 2H), 2.61 (s, 3H), 2.37 (s, 2H) |
| I-4 | 336.1 | — | ¹HNMR (500 MHz, DMSO-d6) δ11.8 (br, 1H), 8.80 |
| | | | (d, J = 8.3 Hz, 1H), 8.00 (t, J = 7.6 Hz, 1H), 7.82 (d, |
| | | | J = 8.3 Hz, 1H), 7.78 (m, 2H), 7.67 (d, J = 7.8 Hz, |
| | | | 1H), 7.61 (t, J = 7.0 Hz, 1H), 7.55 (t, J = 7.4 Hz, |
| | | | 1H), 6.56 (s, 1H), 2.18 (s, 3H) |
| I-5 | 370.1 | — | ¹HNMR (500 MHz, DMSO-d6) δ12.3 (s, 1H), 10.5 |
| | | | (s, 1H), 8.77 (d, J = 8.2 Hz, 1H), 7.92 (m, 2H), 7.85 |
| | | | (m, 3H), 7.56 (t, J = 8.1 Hz, 1H), 7.67 (t, J = 7.4 Hz, |
| | | | 1H), 6.63 (s, 1H), 2.27 (s, 3H); |
| I-6 | 408.2 | — | ¹HNMR (500 MHz, DMSO-d6) δ 1.71 (m, 4H), 1.91 |
| | | | (m, 2H), 3.01 (m, 4H), 7.24 (td, 1H), 7.41 (m, 2H), |
| | | | 7.54 (m, 4H), 10.5 (m, 1H), 13.1 (br. s, 1H); |
| I-7 | 285 | 3.03 | (400 MHz, DMSO) 0.88-0.98 (4H, m), 1.94-2.04 |
| | | | (1H, m), 2.30 (3H, s), 7.30 (1H, s), 8.25-8.35 (1H, |
| | | | m), 8.54-8.62 (1H, m), 10.07 (1H, s), 13.17 (1H, s). |
| I-8 | 284 | 3.19 | (400 MHz. DMSO) 0.83-1.00 (4H, m), 1.93-2.04 |
| | | | (1H, m), 7.18-7.34 (2H, m), 7.42-7.52 (1H, m), 7.68- |
| | | | 7.80 (1H, m), 9.86 (1H, s), 12.54 (1H, s). |
| I-9 | 298 | 3.33 | (400 MHz, DMSO) 1.75-1.87 (1H, m), 1.91-2.05 |
| | | | (1H, m), 2.16-2.26 (1H, m), 2.31-2.45 (5H, m), 3.49- |
| | | | 3.62 (1H, m), 7.19-7.53 (3H, m), 7.75-7.88 (1H, m), |
| | | | 10.02 (1H, s), 12.57 (1H, s). |
| I-10 | 299 | 3.17 | (400 MHz, DMSO) 1.74-1.86 (1H, m), 1.91-2.05 |
| | | | (1H, m), 2.16-2.27 (2H, m), 2.30-2.43 (5H, m), 3.49- |
| | | | 3.62 (1H, m), 7.38 (1H, brs), 8.30-8.41 (1H, m), |
| | | | 8.52-8.62 (1H, m), 10.24 (1H, s), 13.20 (1H, s). |
| I-11 | 312 | 3.48 | (400 MHz, DMSO) 1.52-2.00 (8H, m), 2.32 (3H, s), |
| | | | 3.03-3.15 (1H, m), 7.20-7.54 (3H, m), 7.72-7.87 |
| | | | (1H, m), 9.96 (1H, s), 12.57 (1H, s). |
| I-12 | 313 | 3.32 | (400 MHz, DMSO) 1.52-2.00 (8H, m), 2.33 (3H, s), |
| | | | 3.04-3.16 (1H, m), 7.36 (1H, brs), 8.25-8.41 (1H, |
| | | | m), 8.49-8.62 (1H, m), 10.17 (1H, s), 13.19 (1H, s). |
| I-13 | 379 | 4.01 | (400 MHz, DMSO) 1.63-1.78 (6H, m), 1.84-2.06 |
| | | | (9H, m), 2.34 (3H, s), 7.30 (1H, brs), 8.27-8.39 (1H, |
| | | | m), 8.52-8.62 (1H, m), 10.03 (1H, s), 13.19 (1H, s). |
| I-14 | 378 | 4.07 | (400 MHz, DMSO) 1.64-1.80 (6H, m), 1.94-2.12 |
| | | | (9H, m), 2.32 (3H, s), 7.19-7.55 (3H, m), 7.75-7.87 |
| | | | (1H, m), 9.81 (1H, s), 12.56 (1H, s). |
| I-15 | 371.58 | 3.76 | (DMSO) 1.20-1.39 (3H, m), 1.40 (6H, s), 1.50-1.61 |
| | | | (2H, m), 1.63-1.92 (5H, m), 2.63 (1H, quin), 5.15 |
| | | | (1H, s, OH), 7.14 (1H, br s), 8.33 (1H, dd), 8.56 (1H, |
| | | | dd), 10.12 (1H, s), 13.18 (1H, s). |
| I-16 | 341.57 | 3.63 | (DMSO) 0.87-1.22 (5H, m), 1.40-1-62 (5H, m), 2.04 |
| | | | (3H, s), 2.20 (3H, s), 2.25 (1H, quin), 7.67 (1H, dd), |
| | | | 8.40 (1H, dd), 8.91 (1H, s), 13.13 (1H, s). |
| I-17 | 341 | 3.73 | (400 MHz, DMSO) 1.38-1.81 (10H, m), 1.85-1.97 |
| | | | (1H, m), 2.33 (3H, s), 2.73-2.84 (1H, m), 7.25-7.41 |
| | | | (1H, m), 8.29-8.38 (1H, m), 8.49-8.60 (1H, m), |
| | | | 10.11 (1H.s), 13.15 (1H, s). |
| I-18 | 355 | 3.75 | (400 MHz, DMSO) 1.26-1.67 (10H, m), 1.71-1.83 |
| | | | (2H, m), 2.18 (3H, s), 2.34 (3H, s), 2.55-2.66 (1H, |
| | | | m), 7.75-7.89 (1H, m), 8.50-8.59 (1H, m), 9.00-9.15 |
| | | | (1H, m), 13.26 (1H, s). |
| I-19 | 329 | 2.87 | (400 MHz., DMSO) 1.75-1.85 (4H, m), 2.35 (3H, s), |
| | | | 2.79-2.91 (1H, m), 3.31-3.48 (2H, m), 3.87-3.97 |
| | | | (2H, m), 7.40 (1H, brs), 8.24-8.39 (1H, m), 8.54- |
| | | | 8.63 (1H, m), 10.21 (1H, s), 13.22 (1H, s). |
| I-20 | 363 | 3.42 | (400 MHz, DMSO) 1.77-2.14 (8H, m), 2.35 (3H, s), |
| | | | 2.75-2.87 (1H, m), 7.41 (1H, brs), 8.26-8.38 (1H, |
| | | | m), 8.52-8.62 (1H, m), 10.22 (1H, s), 13.22 (1H, s). |
| I-21 | 328 | 3.13 | (400 MHz, DMSO) 1.80-1.97 (4H, m), 2.39 (3H, s), |
| | | | 2.85-2.97 (1H, m), 3.36-3.54 (2H, m), 3.93-4.06 |
| | | | (2H, m), 7.26-7.37 (1H, m), 7.41-7.59 (2H, m), 7.77- |
| | | | 7.90 (1H, m), 10.06 (1H, s), 12.65 (1H, s). |
| I-22 | 343 | 2.98 | (400 MHz, DMSO) 1.44-1.67 (4H, m), 2.18 (3H, s), |
| | | | 2.35 (3H, s), 2.59-2.70 (1H, m), 3.22-3.37 (2H, m), |
| | | | 3.71-3.82 (2H, m), 7.73-7.83 (1H, m), 8.52-8.59 |
| | | | (1H, m), 9.10 (1H, s), 13.33 (1H, s). |
| I-23 | 347 | 3.92 | (400 MHz, DMSO) 1.10-1.96 (10H, m), 2.56-2.69 |
| | | | (1H, m), 7.65 (1H, brs), 8.27-8.39 (1H, m), 8.58 |
| | | | (1H, s), 10.72 (1H, s), 13.34 (1H, s). |
| I-24 | 346 | 4.02 | (400 MHz, DMSO) 1.12-1.96 (10H, m), 2.55-2.69 |
| | | | (1H, m), 7.22-7.32 (1H, m), 7.44-7.92 (3H, m), |
| | | | 10.57 (1H, s), 12.70 (1H, s). |
| I-25 | 397 | 4.02 | (400 MHz, DMSO) 1.12-1.94 (10H, m), 2.42-2.55 |
| | | | (1H, m), 3.46-3.55 (4H, m), 3.65-3.73 (4H, m), 7.00 |
| | | | (1H, brs), 7.18-7.29 (1H, m), 7.38-7.47 (1H, m), |
| | | | 7.85-7.93 (1H, m), 9.67 (1H, s), 12.44 (1H, s). |
| I-26 | 410 | 4.07 | (400 MHz, DMSO) 1.14-1.93 (10H, m), 2.19 (3H, s), |
| | | | 2.32-2.56 (5H, m), 3.49-3.59 (4H, m), 7.00 (1H, |
| | | | brs), 7.18-7.30 (1H, m), 7.36-7.47 (1H, m), 7.83- |
| | | | 7.95 (1H, m), 9.63 (1H, s), 12.44 (1H, s). |
| I-27 | 327.4 | 1.72 | H NMR (500 MHz, MeOD) 8.54 (s, 1 H), 8.22 (s, |
| | | | 1H), 2.80(m, 1H), 1.9-1.1(m, 10H) |
| I-28 | 326.4 | 1.94 | H NMR (500 MHz, DMSO-d6) 13.12 (s, 1H), 11.58 |
| | | | (s, 1H), 7.68 (s, 1H), 7.58 (dd, J = 4.2, 8.9 Hz, 1H), |
| | | | 7.32 (t, J = 9.0 Hz, 1H), 2.80 (s, 1H), 1.92-1.05(m, |
| | | | 10H). |
| I-29 | 341.4 | 1.79 | H NMR (500 MHz, DMSO-d6) 13.73 (s, H), 8.64 (s, |
| | | | 1H), 8.25 (s, 1H), 1.89-1.1(m12, H), 0.85 (d, J = 7.9 |
| | | | Hz, 3H), 0.74 (d, J = 12.7 Hz, 1H). |
| I-30 | 316.17 | 3.88 | (DMSO) 2.19 (3H, s), 2.32 (3H, s), 6.85 (1H, s), |
| | | | 7.00-7.02 (2H, m), 7.43-7.51 (2H, m), 7.60 (1H, d), |
| | | | 7.70 (1H, d), 11.04-11.06 (1H, m). |
| I-31 | 354.39 | 3.74 | (DMSO) 2.38 (3H, s), 6.90 (1H, s), 7.42-7.44 (2H, |
| | | | m), 7.53-7.56 (2H, m), 7.75 (1H, s), 8.39-8.41 (1H, |
| | | | m), 8.51 (1H, dd), 9.87 (1H, s), 12.93 (1H, s). |
| I-32 | — | — | (DMSO) 7.36 (1H, s), 7.48-7.51 (2H, m), 7.60-7.64 |
| | | | (2H, m), 8.31 (1H, s), 8.43 (1H, d), 8.56 (1H, s), |
| | | | 10.61 (1H, s), 13.17 (1H, s). |
| I-33 | 334.35 | 3.87 | (DMSO) 2.11 (3H, s), 2.33 (3H, s), 7.40-7.45 (5H, |
| | | | m), 7.78 (1H, s), 8.39 (1H, d), 8.50 (1H, s), 9.71 |
| | | | (1H, s), 12.85 (1H, s). |
| I-34 | 368.14 | 3.88 | (DMSO) 1.92 (3H, s), 2.33 (3H, s), 7.37-7.45 (3H, |
| | | | m), 7.55-7.57 (1H, m), 7.82 (1H, s), 8.35 (1H, dd), |
| | | | 8.49 (1H, s), 9.81 (1H, s), 12.86 (1H, s). |
| I-35 | 368.25 | 3.93 | (DMSO) 2.26 (3H, s), 2.32 (3H, s), 6.89 (1H, s), |
| | | | 7.27-7.35 (3H, m), 7.45 (1H, d), 7.83 (1H, dd), 8.45 |
| | | | (1H, s), 8.67 (1H, s), 13.07 (1H, s). |
| I-36 | 383.1 | 1.83 | DMSO d6: 1.23(t, 3H), 2.53(s, 3H), 2.85(q, 2H), |
| | | | 7.40(dd, 1H), 7.45(dd, 1H), 7.48(dd, 1H), 7.56(d, |
| | | | 1H), 7.96(dd, 1H), 8.51(s, 1H) |
| I-37 | 369.3 | 1.8 | H NMR (500 MHz, DMSO-d6) 13.67 (s, 1H), 8.54 |
| | | | (s, 1H), 8.02 (d, J = 8.4 Hz, 1H), 7.70-7.43 (m, |
| | | | 4H), 3.57 (s, 3H), 2.36 (s, 3H) |
| I-38 | 403.4 | 1.9 | — |
| I-39 | 471.16 | 3.38 | (DMSO) 2.11 + 2.16 (3H, s, rotameric), 2.65-2.75 + |
| | | | 2.80-2.90 (2H, mrotameric), 3.81-3.87 (2H, m), 4.53 + |
| | | | 4.57 (2H, s, rotameric), 7.19-7.24 (2H, m), 7.45- |
| | | | 7.71 (5H, m), 9.30 + 9.32 (1H, s, rotameric), 12.84 |
| | | | (1H, s). |
| I-40 | 454.9 | 2.03 | H DMSO: 2.98(t, 2H), 3.53(t, 4H), 4.54(s, 2H), |
| | | | 7.34(d, 1H), 7.45(m, 3H), 7.57(d, 1H), 7.70(d, 1H), |
| | | | 7.78(dd, 1H), 10.65(s, br, 1H) |
| I-41 | 381.3 | 2.1 | — |
| I-42 | 415.3 | 2.3 | — |
| I-43 | 272 | 3.4 | (400 MHz, DMSO) 1.15-1.42 (3H, m), 1.48-1.62 |
| | | | (2H, m), 1.65-1.94 (5H, m), 2.21 (3H, s), 2.24 (3H, |
| | | | s), 2.48-2.62 (1H, m), 6.15 (1H, brs), 6.85 (1H, brs), |
| | | | 9.43 (1H, s), 11.88 (1H, s). |
| I-44 | 481.5 | 1.65 | H NMR (500 MHz, Methanol-d4) 8.52 (d, J = 1.6 |
| | | | Hz, 1H), 8.10 (dd, J = 2.7, 8.4 Hz, 1H), 7.74 (d, J = |
| | | | 7.7 Hz, 1H), 7.64-7.61 (m, 2H), 7.55 (d, J = 7.5 |
| | | | Hz, 1H), 3.34-3.29 (m, 4H), 3.18 (q, J = 7.3 Hz, |
| | | | 3H), 3.12-3.09 (m, 2H), 3.05-3.02 (m, 5H), 1.33 |
| | | | (t, J = 7.3 Hz, 3H) |
| I-45 | 456.4 | 1.91 | H NMR (500 MHz, Methanol-d4) 8.48 (dd, J = 1.7, |
| | | | 2.6 Hz, 1H), 8.10 (dd, J = 2.7, 8.5 Hz, 1H), 7.74 (dd, |
| | | | J = 1.7, 7.5 Hz, 1H), 7.62 (s, 1H), 7.56 (dd, J = 1.2, |
| | | | 7.9 Hz, 1H), 7.52-7.43 (m, 2H), 4.49 (s, 2H), 3.73 |
| | | | (t, 2H), 3.70 (t, J = 7.0 Hz, 2H), 3.31-3.23 (m, 4H). |
| I-46 | 442.4 | 1.74 | H NMR (500 MHz, Methanol-d4) 8.46 (dd, J = 1.7, |
| | | | 2.6 Hz, 1H), 8.10 (dd, J = 2.7, 8.5 Hz, 1H), 7.71 (dd, |
| | | | J = 1.7, 7.5 Hz, 1 H), 7.56 (s, 1H), 7.53 (dd, J = 1.2, |
| | | | 7.9 Hz, 1H), 7.47-7.40 (m, 2H), 3.84 (t, J = 5.2 |
| | | | Hz, 3H), 3.67 (t, 2H), 3.36 (d, J = 2.6 Hz, 1H), 3.31- |
| | | | 3.27 (m, 3H), 3.02 (s, 3H). |
| I-47 | 438.5 | 1.84 | H NMR (500 MHz, Methanol-d4) 8.47 (dd, J = 1.8, |
| | | | 2.6 Hz, 1H), 8.10 (dd, J = 2.7, 8.5 Hz, 1H), 7.73 (dd, |
| | | | J = 1.8, 7.4 Hz, 1H), 7.57 (s, 1H), 7.55 (dd, J = 1.2, |
| | | | 7.9 Hz, 1H), 7.49-7.42 (m, 2H), 3.68 (t, J = 7.0 |
| | | | Hz, 2H), 3.35-3.6(m, 4H), 3.31 (qn, J = 1.6 Hz, |
| | | | Methanol-d4), 3.24 (t, J = 7.0 Hz, 3H), 2.11 (s, 4H). |
| I-48 | 510.54 | 1.94 | H NMR (500 MHz, Methanol-d4) 8.46 (dd, J = 1.7, |
| | | | 2.6 Hz, 1H), 8.10 (dd, J = 2.7, 8.5 Hz, 1H), 7.70 (dd, |
| | | | J = 1.7, 7.5 Hz, 1H), 7.56(s, 1H), 7.55 (dd, J = 1.1, |
| | | | 7.9 Hz, 1H), 7.49-7.42 (m, 2H), 3.71 (s, 3H), |
| | | | 3.65(m, 2H), 3.60 (t, J = 7.1 Hz, 2H), 3.31 (qn, J = |
| | | | 1.6 Hz, Methanol-d4), 3.25 (t, J = 7.1 Hz, 2H), |
| | | | 3.20(m, 2H), 2.75 (m, 1H), 2.16 (m, 2H), 1.9 (m, 2H) |
| I-49 | 483.5 | 1.82 | H NMR (500 MHz, Methanol-d4) 8.46 (dd, J = 1.7, |
| | | | 2.6 Hz, 1H), 8.10 (dd, J = 2.7, 8.5 Hz, 1H), 7.72 (dd, |
| | | | J = 1.7, 7.4 Hz, 1H), 7.55 (s, 1H), 7.52 (dd, J = 1.3, |
| | | | 7.9 Hz, 1H), 7.47-7.40 (m, 2H), 4.33 (s, 2H), 3.68 |
| | | | (t, J = 6.9 Hz, 2H), 3.31-3.28 (t, 2H), 3.03 (s, 3H), |
| | | | 2.98 (s, 3 H), 2.94 (s, 3H). |
| I-50 | 412.4 | 1.78 | H NMR (500 MHz, Methanol-d4) 8.46 (dd, J = 1.7, |
| | | | 2.6 Hz, 1H), 8.10 (dd, J = 2.7, 8.5 Hz, 1H), 7.72 (dd, |
| | | | J = 1.7, 7.4 Hz, 1H), 7.55 (s, 1H), 7.52 (dd, J = 1.3, |
| | | | 7.9 Hz, 1H), 7.47-7.40 (m, 2H), 3.62 (t, J = 6.9 Hz, |
| | | | 2H), 3.25 (t, 2H), 3.00 (s, 6H). |
| I-51 | 487.5 | 1.69 | H NMR (500 MHz, Methanol-d4) 8.49 (dd, J = 1.6, |
| | | | 2.5 Hz, 1H), 8.05 (dd, J = 2.7, 8.4 Hz, 1H), 7.90 (d, |
| | | | J = 7.3 Hz, 1H), 7.81-7.76 (m, 4H), 3.31 (qn, J = |
| | | | 1.6 Hz, Methanol-d4), 3.14-3.10 (m, 4H), 3.03 (d, |
| | | | J = 5.2 Hz, 4H). |
| I-52 | 371.37 | 1.94 | (500 MHz, DMSO-d6) 13.15, (s, 1H), 10.4 (s, 1H), |
| | | | 8.5 (m. 1H), 8.45 (d, 1H), 7.85 (m, 1H), 7.75 (m, |
| | | | 1H), 7.65 (m, 3H), 7.15 (m, 1H), 2.45 (s, 3H) ppm |
| I-53 | 354.05 | — | — |
| I-54 | 416.2 | 2.1 | DMSO d6: 1.23(t, 3H), 2.5(s, 3H), 2.84(q, 2H), |
| | | | 7.17(m, 2H), 7.46(m, 1H), 7.58(dd, 1H), 7.64(dd, |
| | | | 1H), 7.69(dd, 1H), 7.74(d, 1H) |
| I-55 | 351.3 | 1.6 | — |

### Example 7

### GSK-3 Inhibition Assay

Compounds of the present invention were screened for their ability to inhibit GSK-3β (AA 1-420) activity using a standard coupled enzyme system (Fox et al., Protein Sci. 1998, 7, 2249). Reactions were carried out in a solution containing 100 mM HEPES (pH 7.5), 10 mM MgCl₂, 25 mM NaCl, 300 μM NADH, 1 mM DTT and 1.5% DMSO. Final substrate concentrations in the assay were 20 μM ATP (Sigma Chemicals, St Louis, Mo.) and 300 μM peptide (American Peptide, Sunnyvale, Calif.). Reactions were carried out at 30° C. and 20 nM GSK-3β. Final concentrations of the components of the coupled enzyme system were 2.5 mM phosphoenolpyruvate, 300 μM NADH, 30 μg/ml pyruvate kinase and 10 μg/ml lactate dehydrogenase.

An assay stock buffer solution was prepared containing all of the reagents listed above with the exception of ATP and the test compound of the present invention. The assay stock buffer solution (175 μl) was incubated in a 96 well plate with 5 μl of the test compound of the present invention at final concentrations spanning 0.002 μM to 30 μM at 30° C. for 10 mM. Typically, a 12-point titration was conducted by preparing serial dilutions (from 10 mM compound stocks) with DMSO of the test compounds of the present invention in daughter plates. The reaction was initiated by the addition of 20 μl of ATP (final concentration 20 μM). Rates of reaction were obtained using a Molecular Devices Spectramax plate reader (Sunnyvale, Calif.) over 10 min at 30° C. The Kᵢ values were determined from the rate data as a function of inhibitor concentration. Compounds described herein were found to inhibit GSK-3.

### Example 8

### GSK-3α and GSK3β p-TYR Inhibition Assay

Compounds are screened for their ability to inhibit the phosphorylation of tyrosine (TYR) residues through the use of western blotting of Jurkat cells dosed with the compounds. The phosphorylation of the specific TYR residues tested are GSK3α TYR 279 and GSK3β TYR 216.

Preparation of Cells and Lysates

Jurkat cells are seeded at a density of 2×10⁵ cells/well in a 12 well dish in starvation media (RPMI+1% FBS+P/S). Following starvation for 16 hours, the compound is dosed into each well at a final DMSO concentration of 0.3% and cells are incubated overnight at 37° C. 5% CO₂. The next day, cells are spun down at 1500 rpm, washed with PBS, and lysed in 100 uL Laemli sample buffer with β-mercaptoethanol.

Western Blot Protocol

15 microliters (uL) of cell lysates are loaded onto a 10% tris-glycine gel and run at 120V for 2 hours or until dye front runs off of the gel. The protein is then transferred onto a PVDF membrane at 100V for 60 minutes. PBST (PBS containing 0.1% Tween 20, such as 1 ml Tween per 1 L of PBS) is then made up and used for all washes and antibody incubations. The blot is blocked in 5% nonfat milk PBST for one hour.

The primary antibody (GSK-3α/β pTYR 279/216 at 1:1000 dilution Upstate cat#05-413) is then added in 5%-nonfat milk PBST overnight at 4° C. with gentle rocking. The blot is then washed in PBST for 5 min. This is then repeated 4 times. A secondary anti-mouse-HRP conjugated antibody (1:5000 dilution) is added for 60 minutes in 5% milk PBST. The blot is then washed in PBST for 5 min. This is also repeated 4 times. 3.0 mL of the developing solution (ECL plus Western Blotting Detection System from Amersham/GE cat# RPN2132) is made and then added. The solution is swirled over the blot for ˜30 sec. The blot is then developed using CL-Xposure clear blue X-ray film. GAPDH expression level is used as a loading control, (GAPDH antibody: santa cruz 25-778) at 1:10000 dilution.

For determination of GSK-3α and GSK-3β pTYR IC50, the density of the respective bands for each protein at specific compound concentration is compared to a no compound DMSO treated control cell sample present on each exposure. IC50 numbers are defined as the concentration of compound in which the density of the GSK-3α or GSK-3β band is 50% of the no compound control.

### Example 9

### β-Catenin Stabilization Protocol

GSK-3 phosphorylation of β-catenin targets it to the proteosome for degradation. Inhibition of GSK-3 results in accumulation of β-catenin in the cytosol of cells which, through interaction with the transcription factor TCF/LEF, translocates to the nucleus and drives the transcription of Wnt-dependent genes. The assay is designed to determine the level of β-catenin dependent TCF/LEF transcriptional activity in a quantitative manner through the use of a β-lactamase reporter assay in Jurkat cells dosed with a compound.

Jurkat β-catenin cells are starved overnight in assay media (1% FBS, 1× Penstrep, RPMI) in the flask. The next day, Jurkat β-catenin cells are seeded in 96-well flat-bottom plates at a density of 50,000 cells/well in assay media in a volume of 100 uL. The compound is added to the well at a final DMSO concentration of 0.75% and incubated at 37° C. overnight. The next day, 20 uL of 6×CCF4 dye is added to the wells and incubated at room temperature for 1-2 hours. Plates are read on the Cytofluor 4000 series multiwell plate reader and the 460/530 ratio is determined. The GSK-3 IC50 for induction of β-catenin is determined by plotting the 460/530 ratio against the concentration of compound (Log scale) and using the equation of the slope to calculate the point at which the ratio is 50% of the maximum effect.

### Example 10

### β-Catenin:GSK-3 Window Results

β-catenin:GSK-3 windows were calculated by dividing the β-catenin IC50 value obtained in Example 9 by the GSK-3α or GSK3β p-TYR IC50 value obtained in Example 8.

The following compounds were found to have a β-catenin:GSK-3α window between 35 and 500 fold: I-4, I-15, I-19 to I-22, I-34 to I-36, I-40, and I-51 to I-54. The following compounds were found to have a β-catenin:GSK-3α window between 500 and 1000 fold: I-2, I-3, I-6, I-12, I-27, I-28, I-31, I-32, I-37 to I-39, I-44, I-46 to I-49, and I-55. The following compounds were found to have a β-catenin:GSK-3α window between 1000 and 2000: I-13, I-17, I-18, I-42, and I-43. The following compounds were found to have a β-catenin:GSK-3α window between 2000 and 6000: I-1, I-5, I-16, I-29, I-33, I-41, I-45, and I-50.

The following compounds were found to have a β-catenin:GSK-3β window between 4 and 25 fold: I-19, I-20, I-22, I-29, I-34, and I-45. The following compounds were found to have a β-catenin:GSK-3β window between 25 and 49 fold: I-18, I-28, I-31, I-35, I-36, I-40, I-44, I-46, I-49, I-51, I-53, and I-54. The following compounds were found to have a β-catenin:GSK-3β window between 50 and 100 fold: I-2 to I-4, I-6, I-15, I-17, I-27, I-32, I-37 to I-39, I-41, I-47, I-48, I-50, and I-52. The following compounds were found to have a β-catenin:GSK-3β window between 100 and 400 fold: I-1, I-5, I-16, I-21, I-33, I-42, I-43, and I-55.

### Example 11

### CRMP2 Phosphorylation Assay

GSK-3 phosphorylation regulates CRMP2 which is involved in the control of axon outgrowth and branching (Yoshimura et al., 2005 Cell, Kim et al. 2006 Neuron). Phosphorylation of CRMP2 by GSK-3 reduces CRMP2 binding to microtubules and thereby reduces axon elongation and branching. Conversely, inhibition of GSK-3, especially at levels that selectively affect TYR residue autophosphorylation, enhances these phenotypes. Compounds are tested in E16 rat hippocampal or cortical neurons to determine the ability to increase the level of axonal branching.

Day 1

Preparation of Cell Plates

1 mg/ml stock of PDL is diluted into 100 μg/mL in DI water. The glass coverslips are coated for at least 1 hour at 37° C. prior to doing the dissection. PDL is aspirated and the plates are rinsed with PBS and air-dried in hood.

Dissociation of E-16 Rat Cortical Cells

Cortical or hippocampal lobes are combined with 9 mL of Base media (Neurobasal+Pen/Strep) and put on ice. 1 mL of 10× trypsin solution is added and the Mixture is swirled gently. The tissue is then digested via incubation in a 37° C. waterbath for 20 minutes. After 20 minutes, 10 μL/mL DNase (100 μL DNase) is added and the mixture is incubated for another 5 minutes.

The cells are spun at 1000 rpm for 1 minute. The enzyme solution is then removed without removing any of the brain fragments sitting on the bottom. The solid is washed 3 times with Wash media (Neurobasal+10% and Pen/Strep). After the third wash, the cells are re-suspended in 5 mL of Culture Media (Neurobasal+B27, L-Glutamine and Pen/Strep). Mechanical dissociation is performed by gently pipetting several times through a flame-narrowed glass pipet, taking care not to make bubbles. The cells are then filtered through a 70 μm cell strainer. The cells are counted in a hemacytometer and seeded at 50,000 cells/well in a 12 well plate. The cells are incubated at 37° C. overnight.

Day 2

Cell Maintenance

The next day, half of the media is changed with fresh Culture Media containing retinoic acid (R^{A}). Compounds are added to desired concentration at final DMSO concentration of 0.3%. Half of the media is changed and fresh compound is added every 3 days. Cells are incubated with compounds for 6 days in culture.

Day 7

Collection of Lysates and Western Blot

Cultures are washed with PBS and lysed directly in 100 uL of Laemli sample buffer with β-mercaptoethanol added.

Western Blot Protocol

7 microliters (uL) of cell lysates are loaded onto a 10% tris-glycine gel and run at 120V for 2 hours or until dye front runs off of the gel. The protein is then transferred onto a PVDF membrane at 100V for 60 minutes. PBST (PBS containing 0.1% Tween 20, such as 1 ml Tween per 1 L of PBS) is then made up and used for all washes and antibody incubations. The blot is blocked in 5% nonfat milk PBST for one hour.

The primary antibody (1:10,000 CRMP2 rabbit polyclonal Abcam #ab36201) is then added in 5%-nonfat milk PBST overnight at 4° C. with gentle rocking. The blot is then washed in PBST for 5 minutes. This is then repeated four times. A secondary anti-mouse-HRP conjugated antibody (1:5000 dilution) is added for 60 minutes in 5% milk PBST. The blot is then washed in PBST for 5 minutes. This is also repeated four times.

3.0 mL of the developing solution (ECL plus Western Blotting Detection System from Amersham/GE cat# RPN2132) is made and then added. The solution is swirled over the blot for ˜30 seconds. The blot is then developed using CL-Xposure clear blue X-ray film. GAPDH expression level is used as a loading control, (GAPDH antibody: santa cruz 25-778) at 1:10000 dilution. The CRMP2 antibody detects both the unphosphorylated form of CRMP2 and the phosphorylated form of CRMP2 (T514) which is the residue phosphorylated by GSK-3 (Kim et al. 2006 Neuron). The IC50 of compounds for pCRMP2 is defined as the concentration of compound in which the density of the supershifted pCRMP2 band is 50% of the no compound control.

Results

Inhibition of GSK-3 phosphorylation of substrate CRMP-2 correlated with inhibition of GSK-3 pTYR in E16 hippocampal neurons treated for seven days with Compound I-37. CRMP-2 is enriched in the growing axon, and un-phosphorylated CRMP-2 binds to microtubules and promotes axonal branching.

### Example 12

### In Vitro Model of Angiogenesis Using HUVEC and Skin Fibroblasts

In addition to neuroplasticity, angiogenesis, the formation of new blood vessels, may participate in the functional recovery from brain injury, such as stroke. A role for GSK-3 has been implicated in driving both proliferation and differentiation of endothelial progenitor cells (EPC) depending on the stage of maturation.

Compounds are screened for the ability to enhance angiogenesis in human umbilical vein endothelial cells (HUVEC). This method, adapted from Nakatsu et al., Microvas. Res. 2003, describes a protocol that recapitulates the major events essential for new vessel growth: budding, cell migration, cell proliferation, lumen formation, branching, and anastomosis.

Protocol:

HUVEC are used between P3 and P4. HUVEC are mixed with dextran coated cytodex 3 micro-carriers (Amersham Pharmacia) at a concentration of 400 HUVEC per bead in 1 ml of EGM-2 (2% FBS) medium (Clonetics). Beads with cells are then shaken gently every 20 minutes for 4 hours at 37° C. and 5% CO₂. After incubating, beads with cells are transferred to a T-25 tissue culture flask and left for 12-16 hours in 5 ml of EGM-2 at 37° C. and 5% CO₂.

The following day, beads with cells are washed three times with EGM-2 and re-suspended at a concentration of 200 cell-coated beads/ml in 2.5 mg/ml of fibrinogen (Sigma) with 0.15 U/ml of aprotinin (Sigma) at a pH of 7.4.

500 uL of fibrinogen/bead solution is then added to 0.625 U of thrombin (Sigma) in one well of a 24 well tissue culture plate. Fibrinogen/Bead solution is allowed to clot for 5 minutes at room temperature and then at 37° C. and 5% CO₂ for 20 minutes. 1 mL of EGM-2 with 0.15 U/mL of aprotinin is then added to each well and equilibrated with the fibrin clot for 30 minutes at 37° C. and 5% CO₂.

Next, media is removed from the well and replaced with 1 mL of fresh medium. 20,000 skin fibroblasts (SF ATCC Detroit 551) are plated on top of the clot and medium is changed every other day.

For compound inhibition studies, a 6 point dose response is performed (1 uM top concentration 1:3 dilutions) in which compound is added to the clot following equilibration.

Angiogenesis is scored by quantification of images captured on an inverted microscope at 10× and 20× magnification for vessel length, number of vessels and branches per bead using NIH Image J software. Optionally, prior to the assay, HUVEC can be spin transduced with a retroviral vector expressing yellow fluorescent protein (YFP) under the control of a constitutively active minimal TK promoter, and sorted for YFP expression to enhance visualization. YFP positive HUVEC are then cultured as described above and quantification of vessel formation is determined by calculating the area under the threshold fluorescence using NIH Image J software. In both cases, enhanced angiogenesis is determined by comparing compound treated cultures with a DMSO control culture at the same time point.

Treatment of HUVEC cultures with Compound I-37 (10 nM) for 7 days results in increase vessel and network formation. When HUVEC cultures were treated at concentrations that have been shown to induce β-catenin, vessel formation was inhibited and increased cell proliferation was observed further supporting a therapeutic role for the window between GSK-3α/β pTYR and β-catenin.

### Example 13

### Leukocytopenia Animal Model

### Definitions

ANC: actual neutrophil count

RBC: red blood cell count

WBC: white blood cell count

5-FU: 5-fluorouracil

rhG-CSF: recombinant human granulocyte colony stimulating factor

This study evaluates the hematopoietic recovery of peripheral blood and bone marrow cells following treatment with 5-FU (a myeloablative reagent) and compares the recoveries of various doses of the test compound with a cytokine known to enhance recovery rhG-CSF. The introduction of the chemotherapeutic agent causes a decrease in the peripheral blood white blood cell count and in particular, the neutrophil count. In addition, there is a significant reduction in the cellularity of the bone marrow as assessed by femoral cell counts. 5-FU acts by killing all the dividing cells, sparing primitive stem cells which are relatively quiescent and are thus not impacted by the treatment. Following treatment, primitive cells are recruited to proliferate and differentiate, thus re-establishing normal peripheral blood and femoral cell counts. This model may be used to evaluate compounds that could facilitate the recruitment and proliferation of the primitive cells and assess the kinetics of recovery of bone marrow as well as mature cell populations (white blood cells, red blood cells and platelets) in the blood.

Animals and Treatment Groups:

One hundred and five C57BL/6 mice at 6-8 weeks of age were purchased from Jackson Labs. All, with the exception of the 4 untreated control mice were treated with 150 mg/kg 5-Fluorouracil (5-FU) interperitoneal (ip) at day 0. The four untreated mice (Group 1) were sacrificed on day 12 and they represent the “normal variability in blood and cell counts”. The 5-FU treated mice were divided equally into 4 distinct groups (Groups 2, 3, 4 and 5). On day −1 (the day before treatment with 5-FU) through to day 12, the mice in group 2 were treated at 8 a.m. and 6 p.m. with just solvent (20% PEG 400/10% Vitamin E TPGS/70% water) and served as the solvent control for groups 4 and 5.

Four mice from this group were sacrificed on days 4, 6, 8, 10 and 12 following the final dose of solvent the morning before sacrificing. On days 1 through 4, the mice in group three were dosed with 50 ug/kg rhG-CSF twice daily with 6 hours separating the daily doses. Four mice from this group were also sacrificed on days 4, 6, 8, 10 and 12. For treatment of test compound, groups 4 and 5, dosing was done as indicated above with the solvent control (2 times daily at 8 am and 6 pm) with compound that was prepared fresh each day.

Compound I-3 was weighed out into 13 vials and an appropriate amount of solvent added to achieve a final concentration for dosing at 30 mg/Kg (high dose). The compound was solubilized by sonication in solvent for 2-3 minutes and then 30% of the volume removed and diluted 1:3 with solvent to prepare compound for dosing at 10 mg/Kg (low dose). This was done daily and delivered to the animal technologist at 1 pm for the evening and next morning dosing. The unused compound was returned and frozen the next day when the freshly prepared sample for the next doses delivered. On days 1 through 12, mice in group 4 received the low dose of compound (10 ug/Kg) and mice in group 5 received the high dose of compound (30 mg/Kg) twice daily. Four mice were sacrificed for each group on days 4, 6, 8, 10 and 12.

Samples and Analyses:

Each time mice were sacrificed from any of the groups, peripheral blood was collected in EDTA microtainer tubes by cardiac puncture. Approximately 400 uL of the blood was sent for analysis of blood indices (WBC, ANC, platelets, hemoglobin and hematocrit) at a central veterinary service laboratory using an automated system. The remainder of the blood was sent to STI where the plasma was separated by centrifugation at 8000 r.p.m. for 10 minutes and stored at −80° C. for future bioassays. Both femurs were also harvested and cells extracted and processed. The femoral cells were counted, red blood cells lysed and the cells subsequently prepared (at 5×10⁶ cells/100 uL in Laemmli buffer and an equivalent unprocessed cell pellet) and stored at −80° C. for future protein analyses. Also on day 12, when the animals were sacrificed, their livers were removed and stored in 10% neutral formalin for future analysis. All mice were sacrificed in accordance with the Canadian Council on Animal Care Guidelines following treatment.

Tests Performed:

Bone Marrow Nucleated Cell Counts

The nucleated cell counts were performed in 3% acetic acid using a Neubauer counting chamber. From these numbers, the total nucleated bone marrow cells per femur were calculated.

Statistical Analyses of CFC numbers:

Statistical analyses were performed on the WBC, ANC, RBC, Platelet counts, hemoglobin and femoral cell content per femur between the groups of mice. A p value of <0.05 was deemed significant where groups of animals were compared. The nadirs and kinetics of cellular recovery for specific populations were assessed and compared between treatment groups.

Results:

Peripheral Blood Analyses:

All of the mice in group 1 (untreated mice) had peripheral blood indices and bone marrow femoral counts along the normal reference ranges as defined by the veterinary clinic, with the exception of a slightly elevated WBC, due to high lymphocytes.

White Blood Cell Counts

In all treatment groups; 5-FU+solvent, 5-FU+rhG-CSF, 5-FU+Compound I-3 (10 mg/Kg), and 5-FU+Compound I-3 (30 mg/Kg) the WBC nadir (lowest cell count observed) was similar between treatment groups and occurred on day 4.

Actual Neutrophil Counts

The actual neutrophil counts (ANC) decreased significantly with time to almost non-detectable levels on days 4 and 6 for mice receiving 5-FU+rhG-CSF. However, the nadir for these mice was earlier (day 4) than for other groups receiving either 5-FU+solvent (day 8) or 5-FU and test compound (day 6), suggesting a possible protective effect in mice receiving solvent.

The kinetics of the WBC and ANC recovery was enhanced in mice receiving 5-FU+test Compound I-3 (high dose), was significantly increased compared to animals receiving 5-FU and solvent control on days 8 and 10, and was comparable to animals receiving 5-FU and rhG-CSF.

Platelet Counts

Platelet counts were modestly decreased and all soared above the reference ranges in groups receiving either 5-FU+rhG-CSF or 5FU+Compound I-3 (high dose) on day 8.

Hemaglobin and Hematocrit Levels

Hemoglobin and hematocrit levels decreased in all animals receiving 5-FU, but there were no differences in levels between groups.

Bone Marrow Cellularity

Bone marrow cellularity was significantly decreased in all treatment groups in response to the 5-FU, with a recovery to normal values on day 10 for mice treated with the high dose of test compound compared to day 12 for all other groups. Therefore, the kinetics of bone marrow cellular recovery was fastest in Group 5 mice, followed by Group 3 and then Group 4. Mice receiving 5-FU and high dose test compound, Compound I-3, did not have as low a nadir as other groups and had significantly higher bone marrow cell counts than mice receiving 5-FU and rhG-CSF on day 8.

Biomarker for Leukocytopenia

To track the activity of Compound (I-3) in mice subjected to 5-FU treatment, peripheral blood was removed at time of sacrifice from both compound and vehicle-treated mice and a protein lysate was obtained from peripheral blood mononuclear cells (PBMC) after removal of red blood cells. Lysates were analyzed by Western blot and probed for GSK-3α/β pTYR levels. Compound I-3 showed a significant reduction in pTYR signal at all doses in PBMC compared to vehicle treated animals with no induction of β-catenin.

### Example 14

### Post Stroke Recovery Model I

This study investigates the effect of compounds on volume of cerebral infarction, cell proliferation, and functional recovery after embolic occlusion of the middle cerebral artery (MCAO) in the rat.

Ten Rats, subjected to MCAO (Zhang R L, et. al., 1997 # 1), were randomly assigned to either Vehicle or Compound I-3 after MCAO. The treatment was administered once daily orally starting 24 hours after MCAo for 14 days. 5-bromo-2-deoxyuridine (BrdU, Sigma; 100 mg/kg, i.p.) is administered at 1 day after MCAO, and consecutively for 6 days. Functional assessments are performed before MCAO and at day 1, day 7 and day 14 after MCAO by an investigator who is blinded to the study. The assessment included Neurological Severity Score (mNSS) and Adhesive removal test score, described below. A total of twenty rats are sacrificed at 14 days after MCAO. Three contralateral hemispheres of each group are assigned for analysis. Volume of infarction and cell proliferation in the ipsilateral subventricular zone (SVZ) are measured.

Adhesive Removal Test:

Rats are tested for left forelimb somatosensory deficit with the adhesive removal test (Schallert T, et. al., 1984 # 3). Each animal received 3 trials by placing round strips of packing tape (1.2 cm in diameter) at each testing day and the mean time (seconds) required to remove stimuli from the left forelimb is was recorded.

Infarction/Ischemic Lesion Volume:

All the animals are anesthetized with ketamine (44 mg/kg IM) and xylazine (13 mg/kg IM) and sacrificed at fourteen days after MCA occlusion. Each rat is transcardially perfused with heparinized saline. The brain is removed from the skull and cut into 7 coronal blocks, each with 2-mm thickness. The brain tissue is processed and embedded, and 6 μm-thick paraffin sections from each block are cut and stained with hematoxylin and eosin (H & E) for evaluation of ischemia cell damage. Infarct volumes are measured using a Global Lab Image analysis program (Data Translation). The area of both hemispheres and the area containing the ischemic neuronal damage (mm²) are calculated by tracing the area on the computer screen. The lesion volume (mm²) is determined by multiplying the appropriate area by the section interval thickness (Chen H, et al., 1992, # 4). The ischemic volume is presented as the percentage of infarct volume of the contralateral hemisphere (indirect volume calculation) (Swanson R A, et al., 1990 #5).

Cell Proliferation in the SVZ:

To visualize specificity of the immunohischemical reaction, immunostaining of BrdU (a monoclonal antibody against BrdU, 1:100, DAK) is employed. The SVZ area is digitized with a 20× objective (BX20 Olympus Optical) using a 3-CCD color video camera (DXC-970 MD; Sony, Tokyo, Japan) interfaced with a MCID image analysis system (Imaging Research) (Chen J, et al., 2005, #6). BrdU-immunoreactive nuclei in the SVZ are counted on a computer monitor to improve visualization. The BrdU data are presented as the number of cells per square millimeter of the SVZ (mean±SE),

### Statistical Analysis

Data are evaluated using Student t-test at the critical value 0.05

Results

Functional Recovery:

To test whether treatment with Compound I-3 improves Functional outcomes after stroke, a battery of functional tests is performed. Results from the functional tests indicated that rats treated with Compound I-3 exhibited significant (p<0.05) improvement in mNSS test and the adhesive removal test compared with vehicle group.

Lesion Volume:

There was no significant reduction in volume of ischemic damage between treatment and vehicle group.

Cell Proliferation in the SVZ:

To test whether treatment with Compound I-1 promotes cell proliferation in the ipsilateral SVZ, BrdU immunostaining is performed. Treatment with Compound I-3 significantly (p<0.05) increased the number of BrdU positive cells in the ipsilateral SVZ compared with the vehicles.

pTYR Biomarker Analysis

To track the activity of Compound I-3 in the CNS of rats subjected to MCAO, brains were removed and protein lysate was obtained as described above from both vehicle treated and compound treated animals at the termination of the study. Lysates were analyzed by Western blot and probed for GSK-3 α/β pTYR levels. Compound I-3 showed a significant reduction in pTYR signal at all doses in the brain compared to vehicle treated rats with no induction of β-catenin.

Conclusion:

The data demonstrates that treatment with Compound I-3 improves functional recovery and promotes cell proliferation in the ipsilateral SVZ after stroke in rats at doses which selectively inhibit GSK-3α/β pTYR.

### Example 15

### Post Stroke Recovery Model II

General Methods

Adult male Wistar rats are pre-trained on a battery of behavioral tests including: tray reach, gridwalk, forelimb asymmetry (cylinder bracing), forelimb inhibition (swim test) (see below for detailed description of tests). Following pre-stroke behavioral assessment, the rats received surgery, during which a stroke is induced. The rats are pseudo-randomly divided into 5 equal groups (n=12) ensuring an equal number of right and left strokes within each treatment group. The first group receives sham surgery with vehicle as treatment. Administration of the test compound and vehicle (dose, route, timing) is determined by the sponsor. The core body temperature is maintained at 37° C. (+/−1°).

Following surgery, all animals are behaviorally assessed 1, 7, and 14 d post-stroke. At the conclusion of behavioral assessment, all rats undergo an MRI to determine infarct volume. Group behavioral performance and stroke volume are compared between groups using one way analysis of variance to determine therapeutic benefit of the compound in rate and extent of recovery of function following MCAO stroke.

Middle Cerebral Artery Occlusion

Each animal is weighed (average weights were 340 g) and then anesthetized with isoflurane (4% isoflurane carried on 21/min medical grade oxygen to induce surgical plane and then 2% with 2 l/min oxygen to maintain a surgical plane). Following induction of anesthesia each rat is individually marked with an ear-punch and administered a subcutaneous dose of buprenorphine (0.025 mg/kg). Rectal temperature is monitored and maintained at 37° C.+/−1° C. for the duration of the surgery and until the rat is awake and mobile (approximately 3 hr).

The rat is then placed into a stereotaxic apparatus positioned such that the lateral aspect of the head was facing up. The skin between the eye left eye and ear is shaved and washed with surgical antiseptic scrub. A vertical incision is made midway between the right orbit and external auditory canal. The underlying temporalis muscle is incised, detached from the skull and retracted with care to preserve the facial nerve. Two sutures hold the temporal muscle away from the lateral aspect of the skull. A craniotomy is performed from the posterior zygoma and along the temporal ridge of the cranium extending ventrally to expose the middle cerebral artery (MCA) and olfactory tract. The dura is opened, and the base of the MCA and the anterior portion of the first branch is electrocoagulated ventral to the olfactory tract, resulting in infarction of the right dorsolateral cerebral cortex.

Time at which stroke occurred is noted upon completion of the electrocoagulation of the MCA. Once bleeding is controlled, the temporal muscle is replaced and the skin sutured. The rat is then removed from the stereotaxic apparatus and moved to the recovery room. A second subcutaneous dose of buprenorphine (0.025 mg/kg) is administered along with 2 cc of Ringers solution. Water, wet rat chow mash, and a warming blanket under 1/2 the cage is made available while in the recovery room. Once the rat is awake and seen to be eating and drinking, it is moved back into its cage in the animal colony.

Methods for Behavioral Testing

Tray Reaching

Forepaw use is measured with a procedure that is adapted from the method devised by Whishaw, O'Connor, and Dunnett (1986). Each animal is food-restricted such that feeding time occurs after testing each day. The animals are placed in test cages (10×18×10 cm high) with floors and fronts constructed of 2-mm bars, 9 mm apart edge to edge. A 4-cm wide and 5-cm deep tray, containing chicken feed pellets, is mounted outside of each box. The rats are required to extend a forelimb through the gap in the bars, grasp, and retract the food.

Animals are trained for 20-30 min per day for a minimum of 10 days, or until a criterion of 50% hits is reached (note: this is 50% for both paws combined). Most rats tend to use exclusively the dominant paw for reaching. If a rat does not reach a criterion of 50% hits within 14 days of training it is excluded from the study. In addition, any rat that appears to be ambidextrous is excluded from the study. Ambidextrous rats use either paw for reaching or reaches equally often with equal success with each paw.

A ‘hit’ is defined as the successful grasping and retrieval of a food pellet that result in consumption of the pellet. A ‘miss’ is defined as the unsuccessful retrieval of a food pellet (either failed to properly grasp pellet, or lost the pellet during the retrieval such that the pellet was not consumed). Percent of hits is calculated as the total number of hits during a session divided by the total number of reaches. This is calculated separately for left and right paw (or affected and unaffected following stoke). Once the criterion of 50% success (involving reaches from both paws) is reached, each rat is video-taped during a 5-min reaching session.

The results of this session serve as pre-surgical baseline. The pre-surgical test session are also be used to determine hand dominance of each rat. The stroke damage is administered within the brain hemisphere that is contralateral to the dominant hand used for reaching. Post surgical testing consists of a 5-min reaching test each week that the animals are tested.

Each session is observed on a monitor using a frame-by-frame analysis of each reach. Each session is scored by 2 different scorers. The final calculation of percent hits for each scorer is within a 5% range of one another. If a greater disparity between final scores occurs then the session is rescored by both observers.

The percentage of hits for affected and non-affected paws for each group is compared among groups using a one-way analysis of variance.

Compound I-37 was tested in the above model. The stroke reliably produced a deficit in reaching performance at 7 and 14 days after surgery. Addition of Compound I-37 significantly ameliorated this deficit at 7 and 14 days after stroke compared with vehicle treatment.

Gridwalk

Forelimb and hindlimb coordination are measured using an apparatus that consists of two Plexiglas panels 1 m long and 25 cm wide (5 mm thick) with holes drilled 1 cm apart along one long edge. The panels are placed 2.5 cm apart and connected via several metal bars (3 mm diameter) through the holes. The bars are randomly placed 1, 2, or 3 cm apart. The apparatus is suspended and oriented such that a narrow alley (2.5 cm wide) is formed 1 m long with walls 25 cm high. The bars form the floor.

Each animal is introduced to the apparatus using 3 trials in which the rat is placed at greater distances from the goal box on each subsequent trial. That is, on the first trial the rat is placed on the end of the grid near and facing the goal box. Once the rat has entered the goal box, it is placed at the half way point on the grid, again facing the goal box. On the third and final trial, the rat is placed at the entrance and allowed to traverse the entire grid to reach the goal box.

This training procedure is done only once for each rat prior to surgery. On all subsequent testing trials, the animals are individually placed at the entrance of the apparatus and required to traverse the entire grid to the goal box.

One test session of 3 trials are conducted before stroke. Each test session after stroke includes 3 trials. Each trial is videotaped at close range from a horizontal plane. The tapes are scored by 2 observers using frame-by-frame analysis. The number of right and left (affected and unaffected) forelimb and hindlimb placement errors through the mid 80% of the grid are counted. The mid 80% of the grid is marked on the outside of the apparatus with masking tape. An error is whenever a limb extends (either partially or fully, i.e., just the paw or the entire leg) through the horizontal plane of the bars. The forelimb and hindlimb errors are summed separately for ipsilateral and contralateral limbs over the three trials and analyzed independently. The scores are compared between groups using one-way analysis of variance.

Compound I-37 was tested in the above model. The stroke reliably produced a deficit in reaching performance at 7 and 14 days after surgery. Compound I-37 significantly ameliorated this deficit at 7 and 14 days after stroke.

Forepaw Asymmetry (Cylinder Test)

Forepaw asymmetry of the animals is measured by placing a rat into a clear acrylic cylinder 25 cm in diameter. The cylinder is placed on a clear table with a mirror positioned such that the animal can be filmed from below. This vantage point provides a clear picture of the animal's forepaws as it explores the cylinder.

During exploration, rats tend to rear a great deal. With each rear, the rat places its forepaws against the side of the cylinder to provide balance and support while investigating the cylinder. Investigation involves leans (while rearing) both to the left and right of the body as well as straight up. A normal rat uses equally both left and right forepaws to brace against the wall. When investigating straight up the wall, the rat uses both paws to brace. During the first 20 rears the bracing paw is noted. The first paw that touches the cylinder wall during the rear is counted. Testing continues until 20 rears have been recorded. The video recording is scored by 2 observers. Left and right paw wall touches are counted. Thus, for each brace, the score could be L or R or L&R.

One test session occurs prior to surgery and then on the designated post-surgical test dates thereafter. The pre-surgery test is used to determine that the rats do not have a preexisting paw bias (that is, more than 15/20 wall touches to one side). If they do show a side bias, they are removed from the study. The post-surgical scores are expressed as percentage of the touches using the affected (contralateral to stroke insult) paw. Groups are compared on this score using one-way analysis of variance.

Compound I-37 was tested in the above model. The stroke reliably produced a deficit in reaching performance at 7 and 14 days after surgery. Compound I-37 at low dose significantly ameliorated this deficit at 14 days after stroke.

Forelimb Inhibition (Swimming Test)

In normal rats, swimming is accomplished by propulsive strokes of the hind limbs. The forelimbs are normally inhibited from any stroking and are held immobile and together under the animal's chin. Inhibition of the forelimbs is assessed by filming animals during swimming. Animals are introduced into one end of an aquarium (30 w×90 1×43 h cm) filled to a depth of 25 cm with room temperature water (−25° C.) and filmed as they swim to a 9.5 cm square visible platform projecting 2 cm above the surface of the water placed at the opposite end. Scoring of inhibition is done by counting the number of left and right forelimb strokes during three placements into the aquarium. Only the mid 80% of the length is scored. The mid 80% is marked on the outside of the aquarium using masking tape.

A swim is deemed scorable only if the animal does not touch the sides of the aquarium during the swimming trial. Groups are compared on the total number of left and right (affected and unaffected following stroke) forelimb strokes using one-way analysis of variance.

Compound I-37 was tested in the above model. The stroke reliably produced a deficit in reaching performance at 7 and 14 days after surgery. Compound I-37 significantly ameliorated this deficit at 7 and 14 days after stroke.

pTYR Biomarker Analysis

To track the activity of Compound I-37 in the CNS of rats subjected to MCAO, brains were removed and protein lysate was obtained (as described herein) from both vehicle treated and compound treated animals at the termination of the study. Lysates were analyzed by Western blot and probed for GSK-3α/β pTYR levels. Compound I-37 showed a significant reduction in pTYR signal at all doses in the brain compared to vehicle treated rats with no induction oβ-catenin.

Adhesive Removal Test

Rats are tested for forelimb somatosensory deficits with the adhesive removal test (Schallert T, et. al., 1984 # 3). Each animal receives 3 trials by placing round strips of packing tape (approx. 1.2 cm in diameter) at each testing day and the mean time (seconds) required to remove stimuli from the left forelimb is recorded.

Compound I-37 was tested in the above model and did not show significant effect.

Methods for Molecular and Histological Analysis

Molecular Analysis

Protein lysate is obtained from the brains of all vehicle and compound-treated animals and is processed for biomarker analysis of GSK3α/β pTYR and β-catenin by Western blot assay to ensure compound activity on the target.

Cerebral spinal fluid is obtained from all vehicle and compound-treated animals and is analyzed for BDNF levels by ELISA as a surrogate marker for neuronal plasticity.

Histological Analysis

Paraffin-embedded brain samples are obtained from Neuroinvestigations and cut into 6 um sections onto glass slides and analyzed by immunohistochemistry or immunofluorescence for markers/phenotypes that correlate with beneficial outcomes in post-stroke recovery:
Stem cell mobilization/proliferation: staining for BrdU and analysis using the Aperio system for quantitation of BrdU positive cells in the subventricular zone (SVZ).
Neurogenesis: immunofluorescent staining for doublecortin (DCX) in conjunction with BrdU in the SVZ using manual counting for quantitation.
Angiogenesis: staining for von Willebrand factor VIII (vWF) and analysis using the Aperio system for quantitation of vWF positive cells in the peri-infarct.

### Example 16

### Axonal Branching Assay

Compounds are tested for the ability to enhance axonal branching in E16 rat hippocampal or cortical neurons.

Day 1

Preparation of Cell Plates

1 mg/ml stock of PDL is diluted into 100 μg/ml in DI water. The glass coverslips are coated for at least 1 hour at 37° C. prior to doing the dissection. PDL is aspirated and the plates are rinsed with PBS and air-dried in hood.

Dissociation of E-16 Rat Cortical Cells

Cortical or hippocampal lobes are combined with 9 mL of Base media (Neurobasal+Pen/Strep) and put on ice. 1 mL of 10× trypsin solution is added and the mixture is swirled gently. The tissue is then digested via incubation in a 37° C. waterbath for 20 minutes. After 20 minutes, 10 μl/ml DNase (100 μL DNase) is added and the mixture is incubated for another 5 minutes.

The cells are spun at 1000 rpm for 1 minute. The enzyme solution is then removed without removing any of the brain fragments sitting on the bottom. The solid is washed 3 times with Wash media (Neurobasal+10% and Pen/Strep). After the 3^{rd} wash, the cells are re-suspended in 5 ml of Culture Media (Neurobasal+B27, L-Glutamine and Pen/Strep). Mechanical dissociation is performed by gently pipetting several times through a flame-narrowed glass pipet, taking care not to make bubbles. The cells are then filtered through a 70 μm cell strainer. The cells are counted in a hemacytometer and seeded at 5000-10000 cells/well in a 24 well plate with glass coverslip inserts coated with PDL. The cells are incubated at 37° C. o/n.

Day 2

Cell Maintenance

The next day, half of the media is changed with fresh Culture Media containing retinoic acid (RA). Compounds are added to desired concentration at final DMSO concentration of 0.3%. Half of the media is changed and fresh compound is added every 3 days. Cells are incubated with compounds for 6 days in culture.

Day 7

Fixation and Staining

### Materials:

1. Phosphate Buffered Saline (PBS)— Gibco 14190-144
2. Wash buffer=PBS-T:
   PBS
   0.1% Tween-20 (Bio Rad, 170-6531)
3. Blocking buffer=PBS-T+5% normal donkey serum or HBSS-T+5% normal donkey serum
   10 ml of PBS
   0.1% Tween-20 (Bio Rad, 170-6531)
   0.5 ml of normal donkey serum (Jackson Immuno # 017-000-121)
5. Gel Mount Citi-Fluor™ (Ted Pella AF-1)
6. Neurofilament antibody 1:250 Abcam, MAP2 antibody 1:250 Abcam
7. Secondary antibody 1:500 for anti-rabbit Alexa 488 (neurofilament) and anti-mouse Alexa 568 (MAP2)

Methods

Cells are washed twice with PBS if the media contains serum. No wash is required if cells are grown in serum-free media.

Fixation

The media or PBS is first removed. Then, 500 uL of HistoChoice is added to cover the cells. The cells are incubated at room temperature for 10 minutes. They are then washed 2 times with PBS, with a 5 minute incubation after each wash. Amounts are shown below:

100 ul of PBS per well in 96 well format

200 ul of PBS per well in 48 well format

400 ul of PBS per well in 24 well format

The cells are incubated with blocking buffer for 30 minutes at room temperature. The tissue is then incubated with blocking buffer for 1 hour at room temperature. 1° antibodies are diluted in PBS+0.1% Tween+5% Donkey serum. The blocking solution is removed and sufficient volume of 1° antibody in blocking buffer is added to cover the cells. 1° antibody is incubated at 4° C. overnight. The next day, 1° antibody is removed and coverslips are washed twice with PBS-T with a 5 minute incubation between each wash. The PBS-T is removed and blocking buffer is added. The cells are incubated for 30 minutes.

The 2° antibody is diluted in PBS+0.1% Tween+5% Donkey serum. The mixture is incubated for 30 mins at room temperature. The slides are washed three times with PBS-T and once with PBS. Mounting media is added to reduce quenching of fluorochromes. The glass coverslips are removed and placed on a slide for visualization.

Analysis

Images are captured at 10× and 20× on an upright microscope and axonal branching is determined by quantification of area under threshold fluorescence of neurofilament Alexa 488 per cell. Dendritic branching is determined by quantification of area under threshold fluorescence of MAP2 Alexa 568 per cell. Alternatively, branching can be determined by manual counting of branch points per cell. Compound effects are assessed by comparing the area under threshold fluorescence in compound treated cultures to that of a DMSO control at the same time point.

Treatment of E16 hippocampal neurons with 10 nM of Compound I-37 for 7 days resulted in increased axonal and dentritic branching. When E16 hippocampal neurons were treated at concentrations that have been shown to induce β-catenin, axon growth was inhibited further supporting a therapeutic role for the window between GSK-3 α/β pTYR and β-catenin

### Example 17

### Spinal Cord Injury Model

Compound I-37 was tested in a spinal cord injury model similar to the one described in Dill, et al, (2008) “Inactivation of Glycogen Synthase Kinase 3 Promotes Axonal Growth and Recover in CNS” The Journal of Neuroscience, 28(36): 8914-8928. Compound I-37 did not appear to improve functional recovery, although the reasons for this observation are unclear.

### Other Embodiments

It is to be understood that, while the invention has been described in conjunction with the detailed description thereof, the foregoing description is intended to illustrate and not limit the scope of the invention, which is defined by the scope of the appended claims. Other aspects, advantages and modifications are within the scope of the following claims.

All references cited herein are incorporated herein by reference in their entirety.

We claim the following:

1. A method of treating a GSK-3 mediated condition, comprising administering to a patient a therapeutically effective amount of a compound of formula I 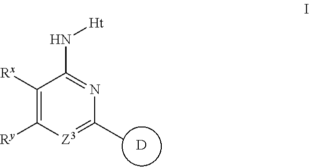
or a pharmaceutically acceptable salt thereof, wherein:
Ht is 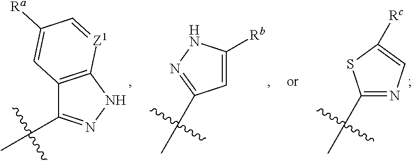
Ring D is phenyl, a 3-8 membered monocyclic cycloaliphatic, a 5-8 membered monocyclic heterocycloaliphatic containing 1-2 heteroatoms and bound to the pyridine or pyrimidine ring via a carbon ring atom, adamantyl, or an 8-10 membered bicyclic cycloaliphatic, wherein the phenyl, heterocycloaliphatic, monocyclic, bicyclic or cycloaliphatic is optionally substituted with 1-2 of —R⁵;
R^{a} is H or halogen;
R^{b} is H or C₁₋₄ alkyl;
R^{c} is H or C₁₋₄ alkyl;
Z¹ is N or CH;
Z³ is N or CR^{Z};
R^{X} is H or C₁₋₄ alkyl;
R^{Y} is H, halogen, a 4-8 membered monocyclic non-aromatic heterocyclyl optionally substituted with one R¹⁰, or C₁₋₄ alkyl optionally substituted with NR₁R₂, 1-3 halo, —OR, or a 4-8 membered monocyclic non-aromatic heterocyclyl containing 1-2 heteroatoms selected from O, N, or S and being optionally substituted with —R¹⁰, or
R^{X} and R^{Y} together with the atoms to which they are bound form phenyl, a 6 to 8 membered cycloaliphatic, or a 5-8 membered monocyclic heterocyclyl containing 1-2 heteroatoms selected from O, N, or S;
R² is H or C₁₋₄ alkyl;
R¹ is H or C₁₋₄ alkyl;
R² is H or C₁₋₄ alkyl optionally substituted with —R¹¹;
Each R⁵ is independently C₁₋₆ alkyl, haloC₁₋₆alkyl, or halo;
Each R¹⁰ is independently selected from C₁₋₆ alkyl, haloC₁₋₆, alkyl, halo, OR, C(═O)R, CO₂R, S(O)R, SO₂R, SR, N(R⁴)₂, CON(R⁴)₂, SO₂N(R⁴)₂, OC(═O)R, N(R⁴)COR, or N(R⁴)CO₂R;
Each R¹¹ is independently selected from halo, OR, C(═O)R, CO₂R, N(R⁴)₂, CON(R⁴)₂, OC(═O)R, N(R⁴)COR, or N(R⁴)CO₂R;
Each R⁴ is independently selected from H, C₁₋₆ alkyl, or haloC₁₋₆ alkyl; and
Each R is independently selected from H, C₁₋₆ alkyl, or haloC₁₋₆ alkyl.

2. The method of claim 1, wherein Ht is

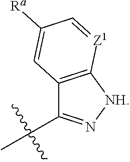

3. The method of claim 2, wherein Z¹ is N.

4. The method of claim 2, where in Z¹ is CH.

5. The method of claim 2, wherein R^{a} is halogen.

6. The method of claim 5, wherein R^{a} is F.

7. The method of claim 1, wherein Ht is

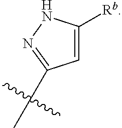

8. The method of claim 7, wherein R^{b} is H.

9. The method of claim 7, wherein R^{b} is CH₃.

10. The method of claim 1, wherein Ht is

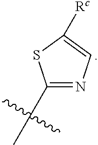

11. The method of claim 10, wherein R^{c} is H.

12. The method of claim 10, wherein R^{c} is CH₃.

13. The method of claim 1, wherein ring D is phenyl optionally substituted with —R⁵.

14. The method of claim 13, where in the phenyl is substituted with —R⁵.

15. The method of claim 14, wherein the phenyl is substituted ortho relative to the attachment to the pyridine or pyrimidine ring.

16. The method of claim 15, wherein the phenyl is substituted with halogen.

17. The method of claim 16, wherein the phenyl is substituted with Cl.

18. The method of claim 14, wherein the phenyl is substituted with C₁₋₆ alkyl.

19. The method of claim 18, wherein the phenyl is substituted with CH₃.

20. The method of claim 14, wherein the phenyl is substituted with haloC₁₋₆ alkyl.

21. The method of claim 20, wherein the phenyl is substituted with CF₃.

22. The method of claim 1, wherein Ring D is a 3-8 membered monocyclic or 8-10 membered bicyclic cycloaliphatic, wherein cycloaliphatic is optionally substituted with —R⁵.

23. The method of claim 22, wherein, Ring D is a 3-8 membered monocyclic cycloaliphatic.

24. The method of claim 22, wherein Ring D is a 8-10 membered bicyclic cycloaliphatic.

25. The method of claim 1, wherein Ring D is a 5-8 membered monocyclic heterocyclic.

26. The method of claim 25, wherein Ring D is tetrahydropyranyl.

27. The method of claim 1, wherein Ring D is adamantyl.

28. The method of claim 1, wherein R^{Y} is C₁₋₄ alkyl optionally substituted with NR₁R₂ or C₁₋₄ alkyl optionally substituted with a 4-8 membered monocyclic non-aromatic heterocyclyl optionally substituted with —R¹⁰.

29. The method of claim 28, wherein R^{Y} is CH₃.

30. The method of claim 28, wherein R^{Y} is ethyl optionally substituted with NR₁R₂ or ethyl optionally substituted with a 4-8 membered monocyclic non-aromatic heterocyclyl optionally substituted with —R¹⁰.

31. The method of claim 30, wherein R^{Y} is —CH₂—CH₂—NR₁R₂.

32. The method of claim 30, wherein R^{Y} is —CH₂—CH₂-(4-8 membered monocyclic non-aromatic heterocyclyl) optionally substituted with —R¹⁰.

33. The method of claim 1, wherein R^{X} is H or CH₃.

34. The method of claim 33, wherein R^{X} is CH₃.

35. The method of claim 1, wherein R^{X} and R^{Y} together with the atoms to which they are bound form phenyl.

36. The method of claim 1, wherein R^{X} and R^{Y} together with the atoms to which they are bound form a 6 to 8 membered cycloaliphatic.

37. The method of claim 1, wherein R^{X} and R^{Y} together with the atoms to which they are bound form a 5 to 8 membered heterocycle.

38. The method of claim 1, wherein the compound is selected from

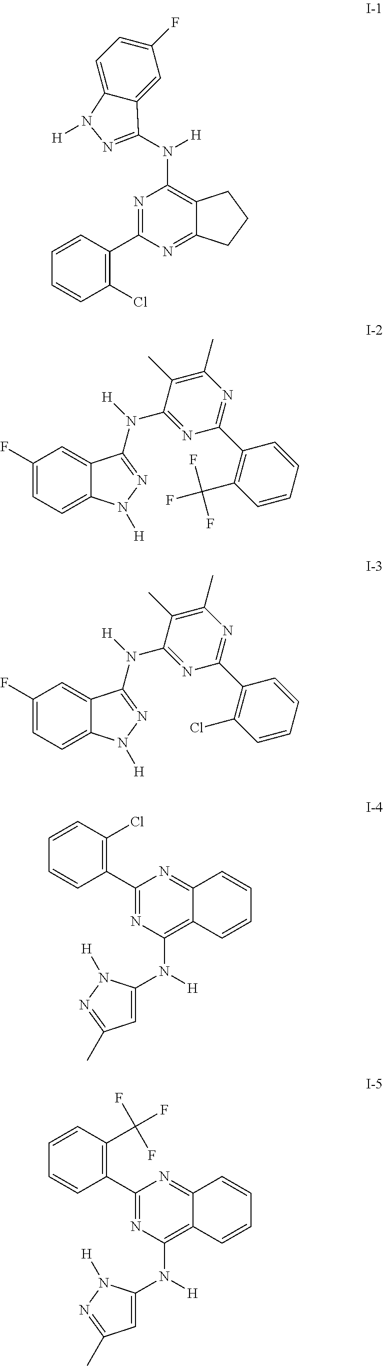

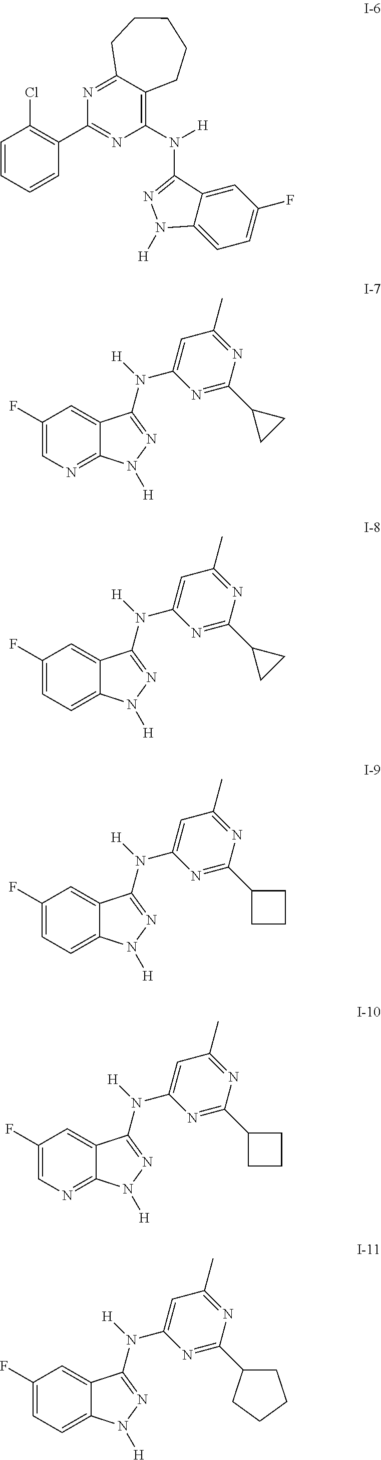

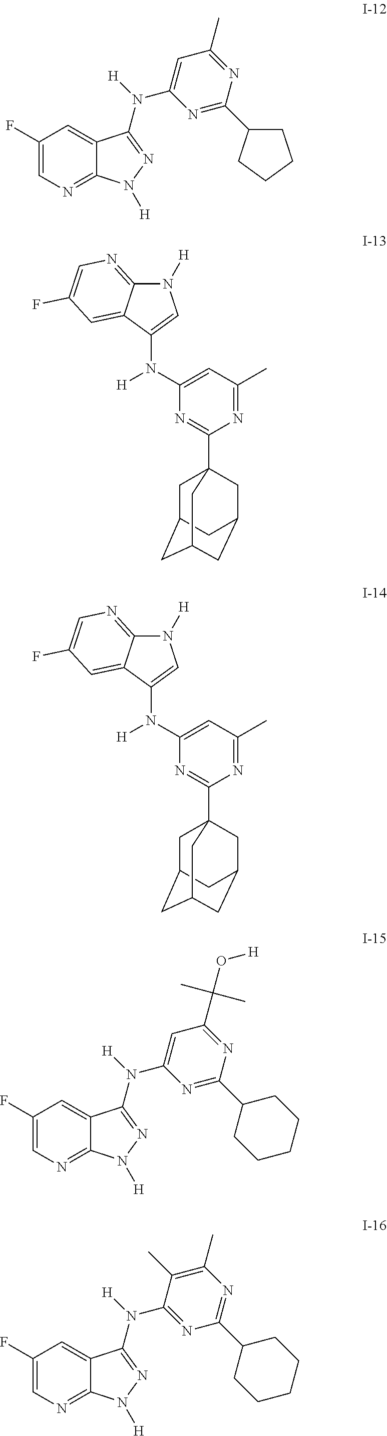

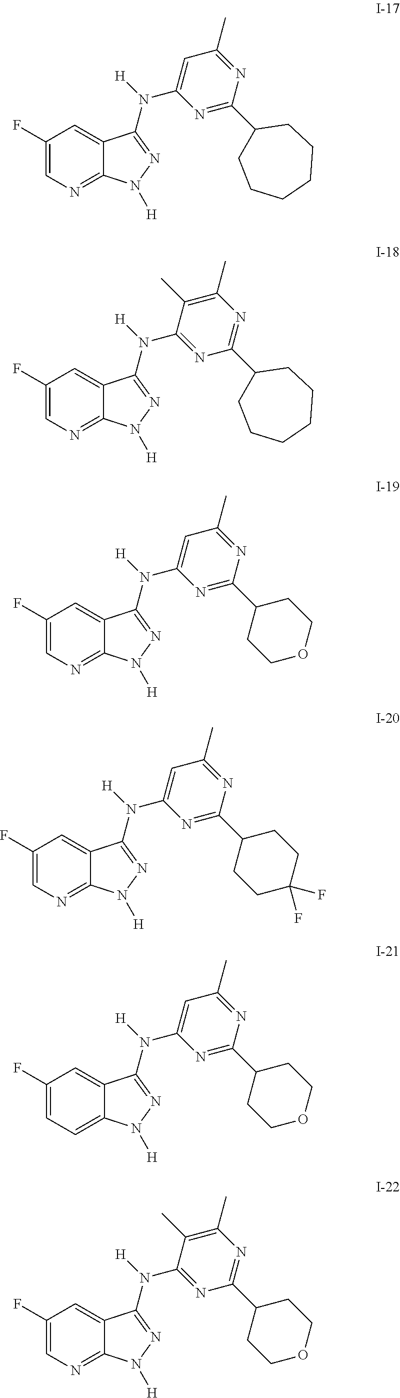

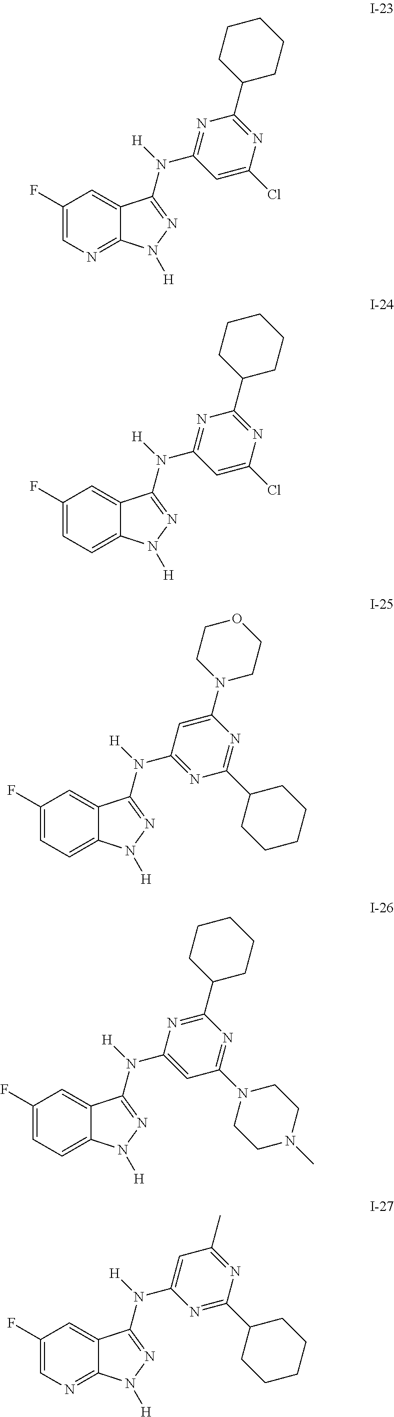

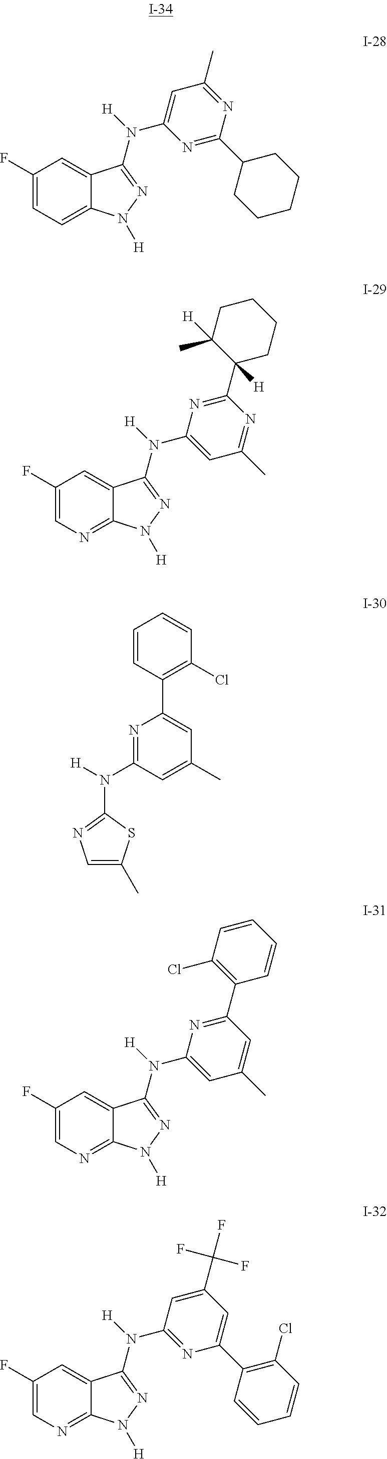

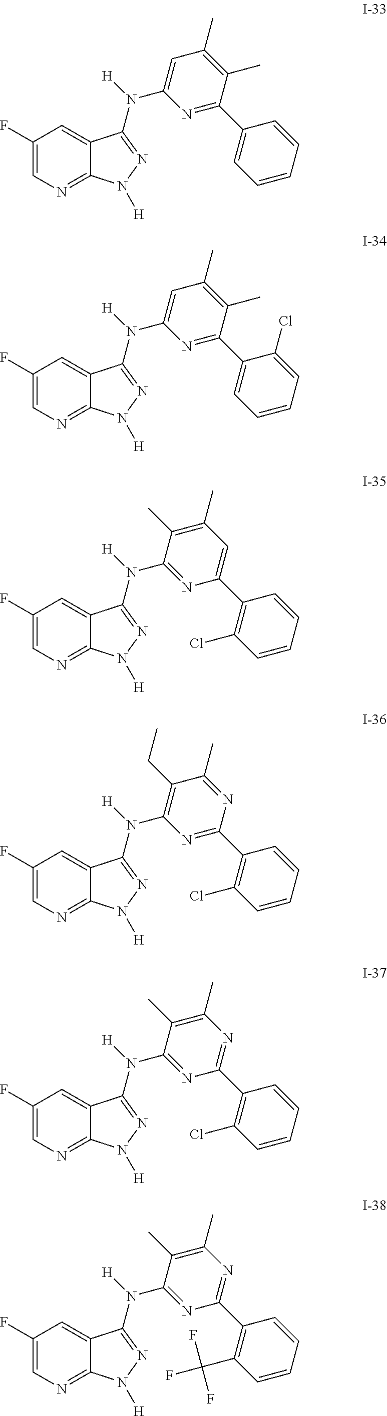

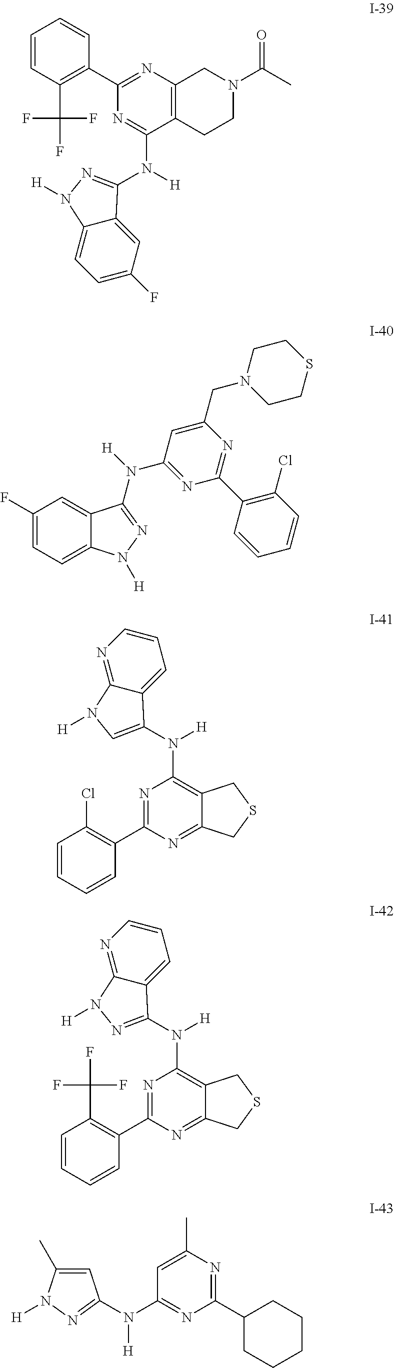

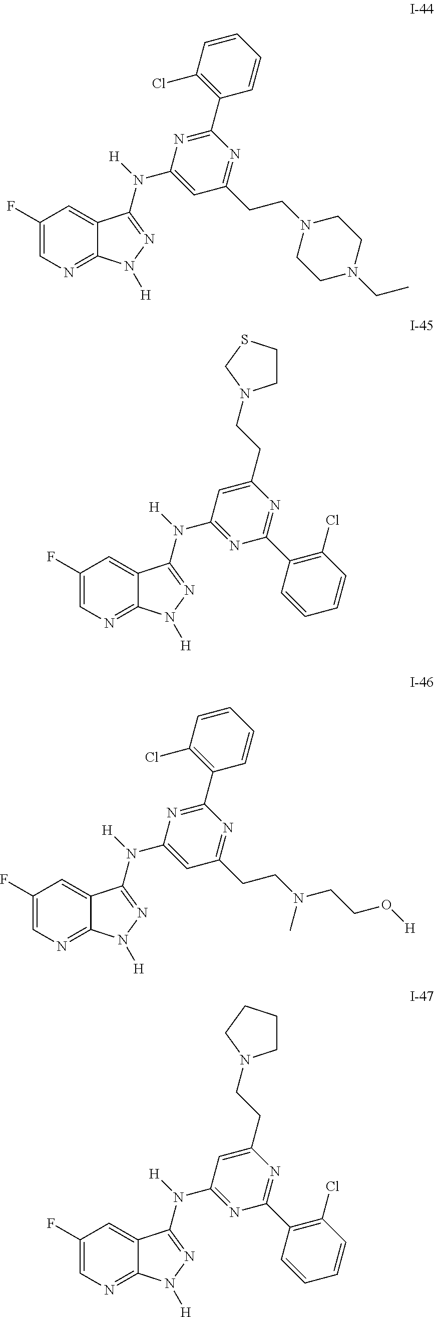

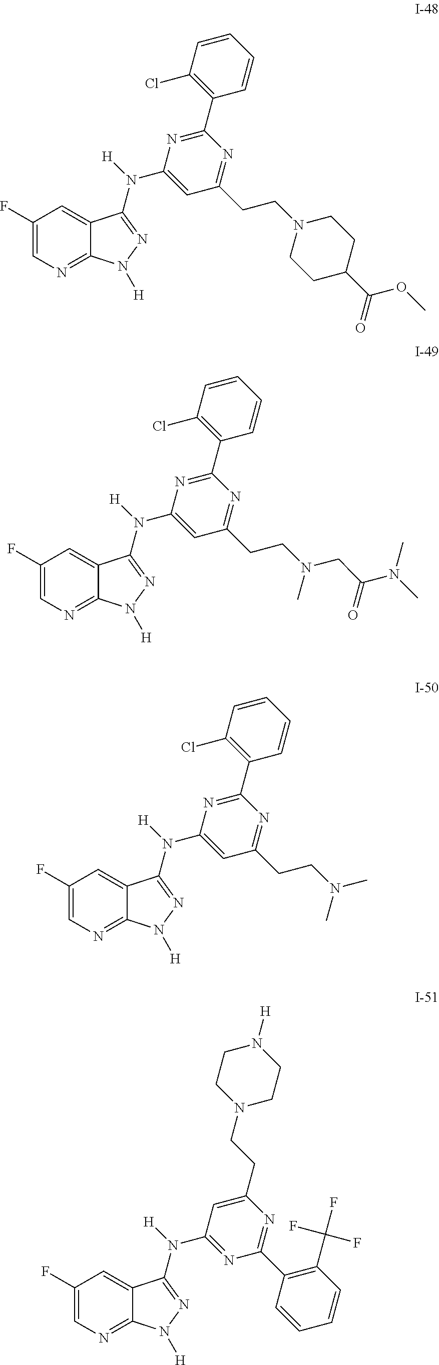

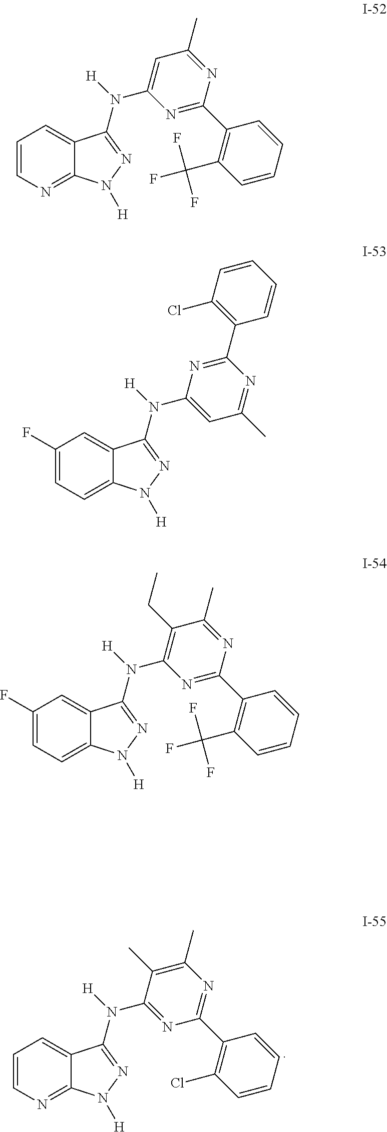

39. The method of claim 1, wherein the GSK-3 mediated condition is treated by inhibiting the GSK-3 activity in an ex vivo or in vitro biological sample.

40. The method of claim 1, wherein the GSK-3 mediated condition is selected from diabetes, osteoporosis, Alzheimer's disease, Huntington's disease, Parkinson's disease, AIDS-associated dementia, bipolar disorder, amyotrophic lateral sclerosis, multiple sclerosis, schizophrenia, leukocytopenia, stroke, neurological disorders, peripheral nerve regeneration, and rheumatoid arthritis.

41. The method of claim 40, wherein said disease is stroke.

42. The method of claim 40, wherein said disease is diabetes.

43. The method of claim 40, wherein said disease is schizophrenia.

44. The method of claim 40, wherein said disease is bipolar disorder.

45. The method of claim 40, wherein said disease is leukocytopenia.

46. The method of claim 40, wherein said disease is selected from stroke, spinal cord injury, traumatic brain injury, Charcot-Marie-Tooth, and diabetic neuropathy.

47. (canceled)

48. The method of claim 41, wherein the compound is administered after ischemia has occurred.

49. The method of claim 40, comprising the additional step of administering to said patient an additional therapeutic agent selected from an agent for treating diabetes, agent for treating osteoporosis, an agent for treating Alzheimer's disease, an agent for treating Huntington's disease, an agent for treating Parkinson's disease, an agent for treating AIDS-associated dementia, an agent for treating bipolar disorder, an agent for treating amyotrophic lateral sclerosis, an agent for treating multiple sclerosis, an agent for treating schizophrenia, an agent for treating leukocytopenia, an agent for treating peripheral nerve regeneration, an agent for treating stroke, and an agent for treating rheumatoid arthritis, wherein:
a) said additional therapeutic agent is appropriate for the disease being treated; and
b) said additional therapeutic agent is administered together with said composition as a single dosage form or separately from said composition as part of a multiple dosage form.

50. The method of claim 49 comprising the additional step of administering to said patient an additional therapeutic agent selected from an agent for treating spinal cord injury, an agent for treating traumatic brain injury, an agent for treating Charcot-Marie-Tooth, or an agent for treating diabetic neuropathy.

51. A method for treating a GSK-3 mediated condition comprising administering an agent comprising a compound selected from

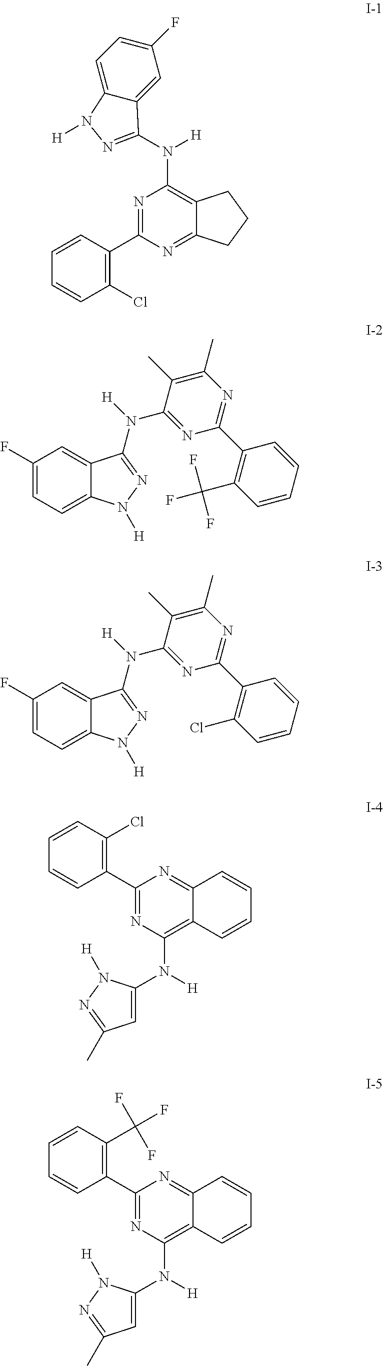

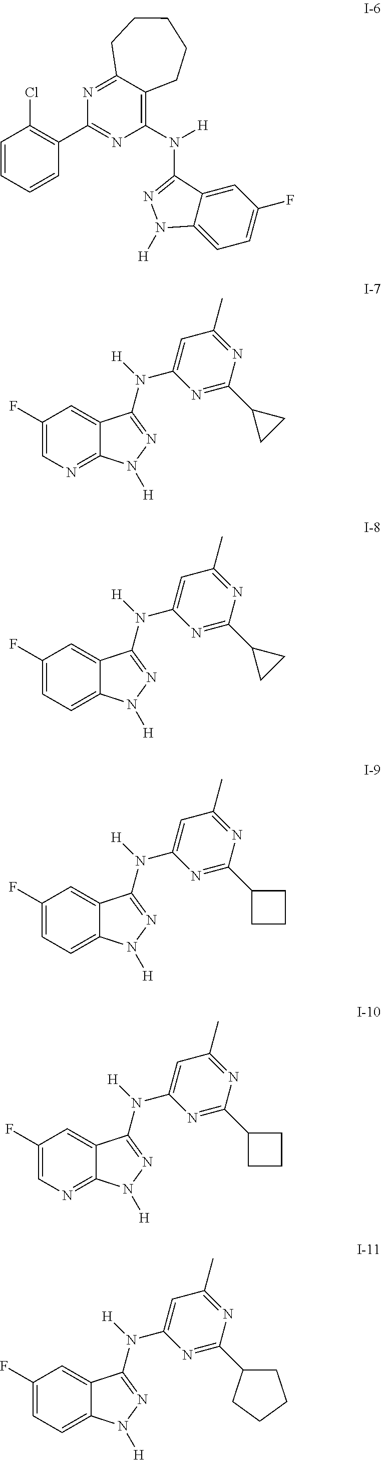

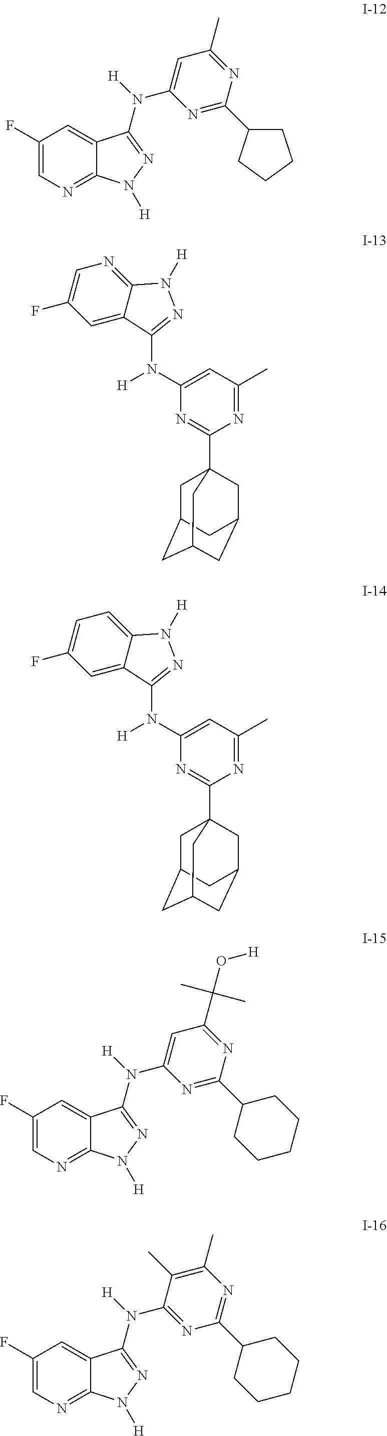

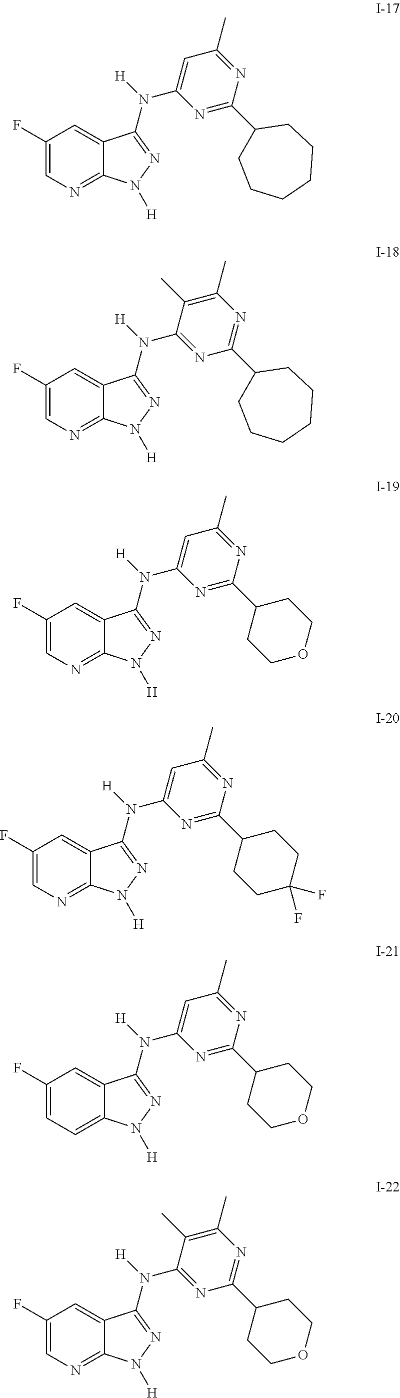

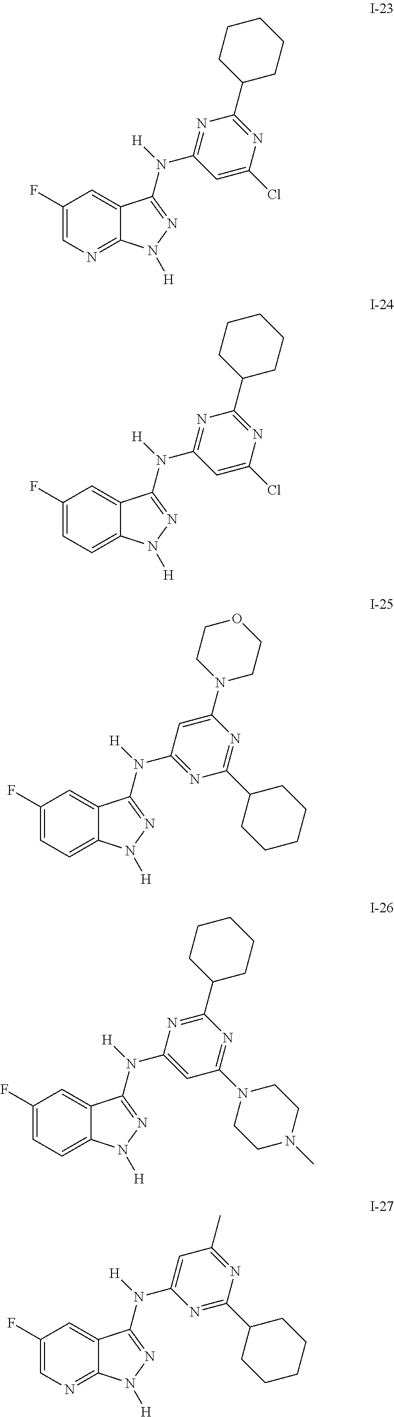

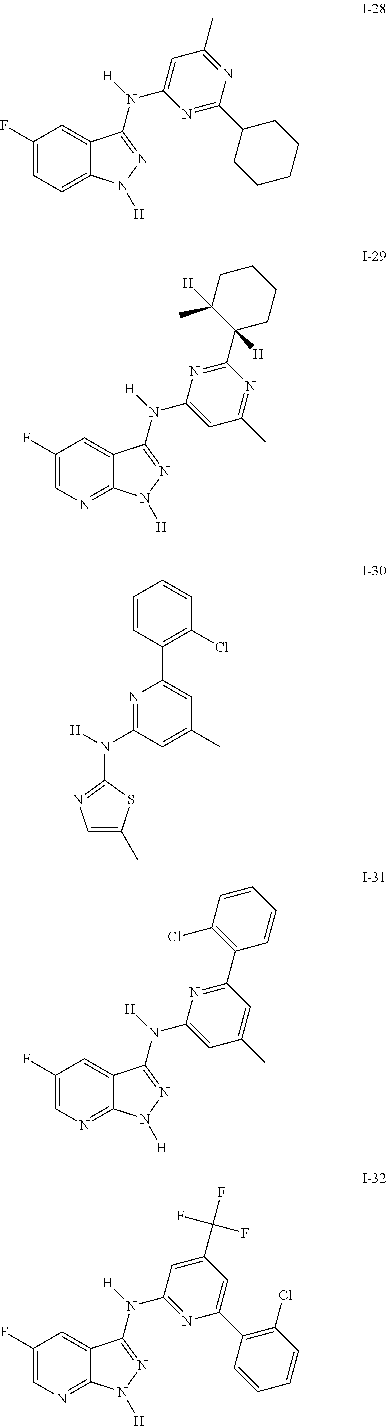

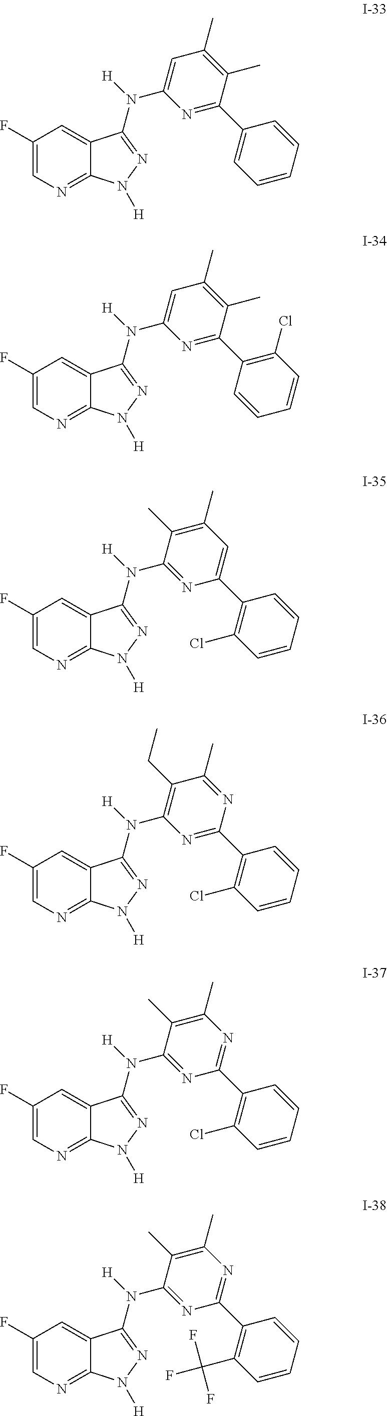

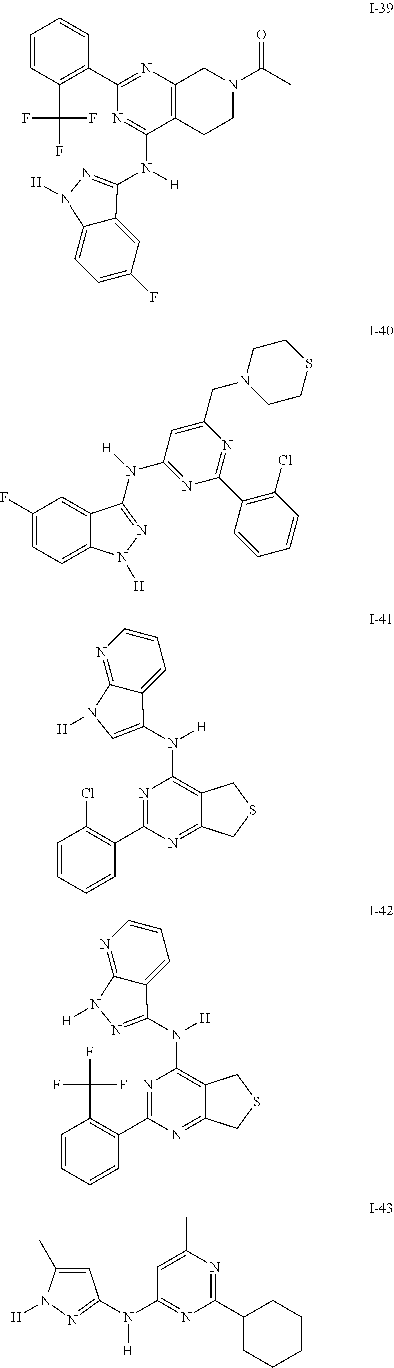

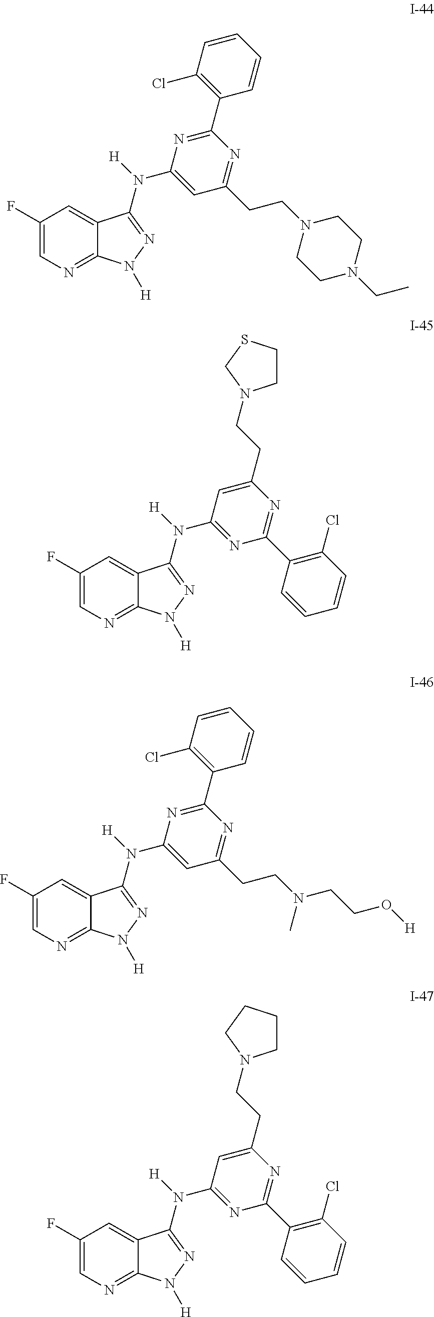

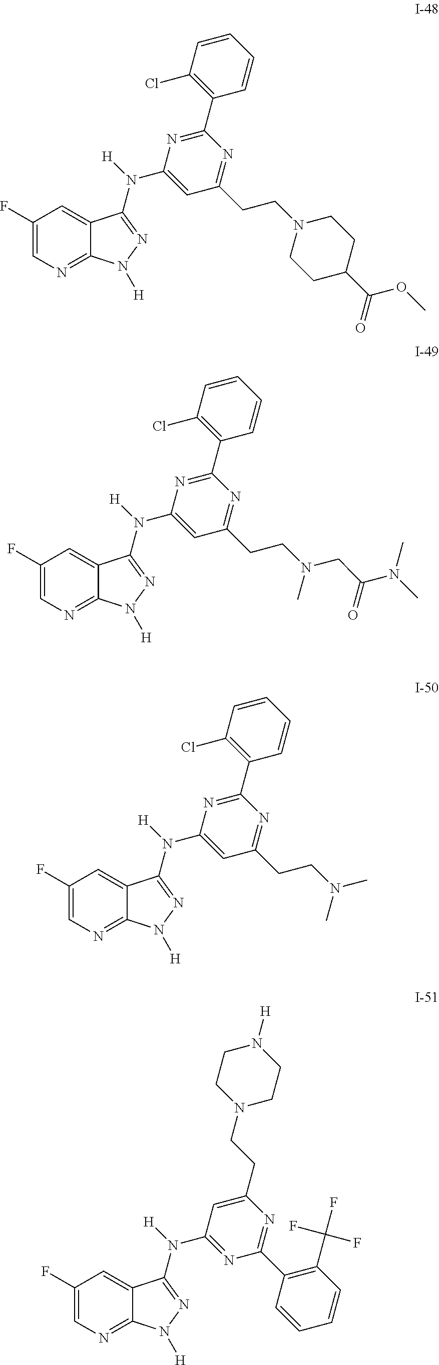

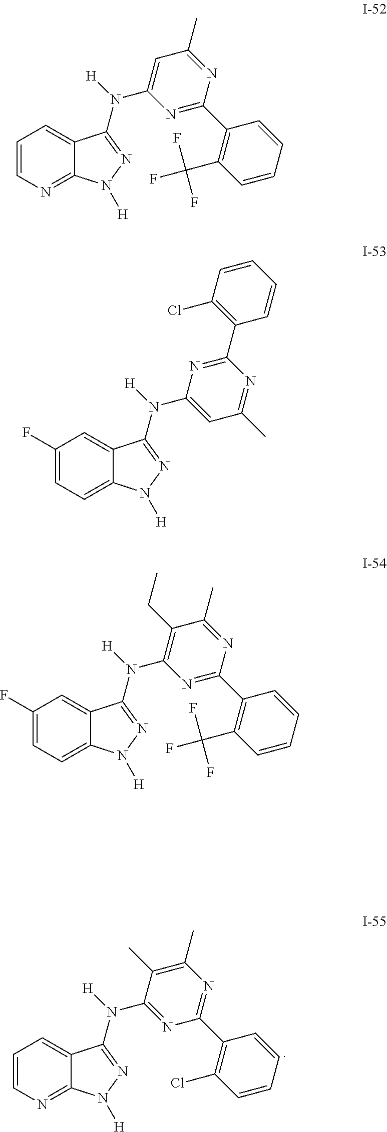

52. (canceled)

53. The method of claim 51, wherein the GSK-3 mediated condition is Post-Stroke, Spinal Cord Injury, Traumatic Brain Injury, Alzheimers, Parkinsons, Huntington, Multiple Sclerosis, Amyotrophic Lateral Sclerosis, Diabetic Neuropathy, Charcot-Marie-Tooth, Leukocytopenia, Diabetes, Peripheral Nerve Regeneration, or Osteoporosis.

54. The method of claim 53, wherein the GSK-3 mediated condition is Post-Stroke.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A method for identifying compounds useful for the treatment of GSK-3-mediated conditions comprising:
measuring the amount of auto-phosphorylation of the tyrosine of the GSK-3 enzyme relative to the serine/threonine kinase form for one or more test compounds.

67. A method for identifying compounds useful for the treatment of GSK-3-mediated conditions comprising:
measuring the amount of auto-phosphorylation of the tyrosine of the GSK-3 enzyme and
measuring the amount of phosphorylation of β-catenin.

68. The method of claim 67, wherein the step of measuring comprises obtaining the β-catenin IC50 value for the test compound, determining the GSK-3α or GSK3β p-TYR IC50 value, and dividing the β-catenin IC50 value by the GSK-3α or GSK3β p-TYR IC50 value.

69. (canceled)

70. (canceled)

71. (canceled)

72. (canceled)

73. (canceled)

74. (canceled)

75. (canceled)

76. (canceled)

77. (canceled)

78. (canceled)

79. A compound selected from 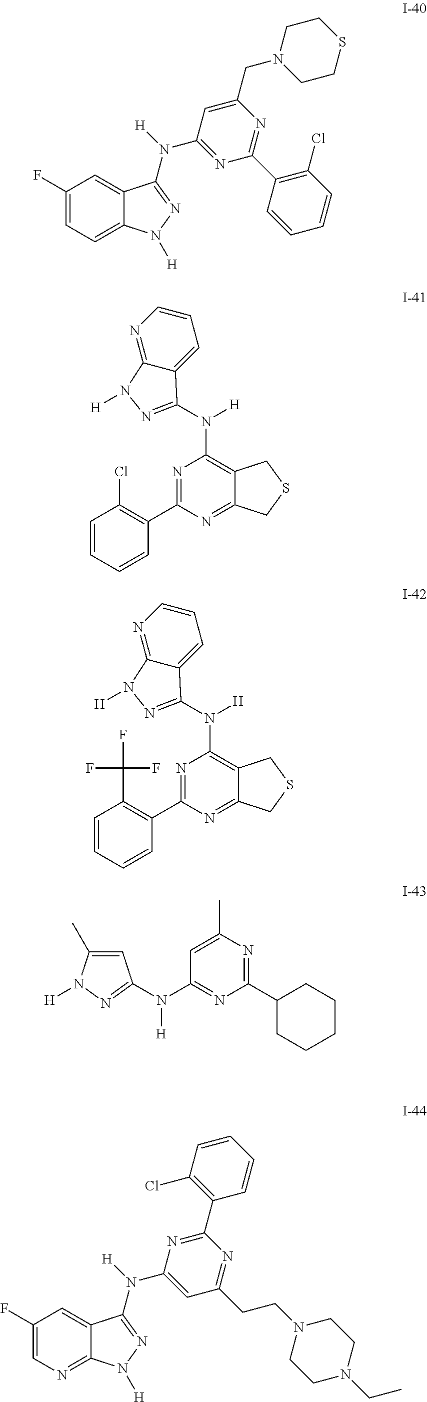 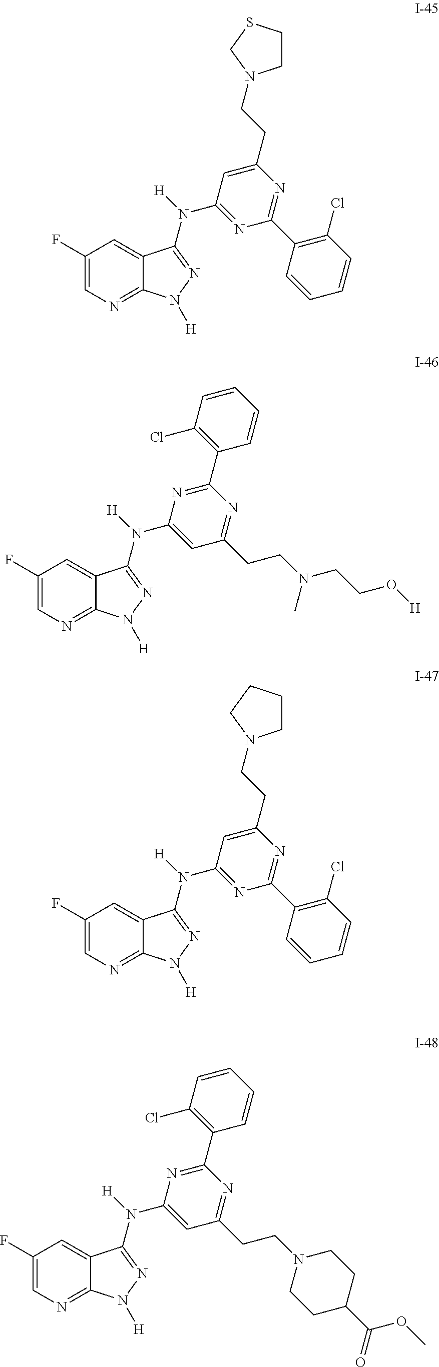 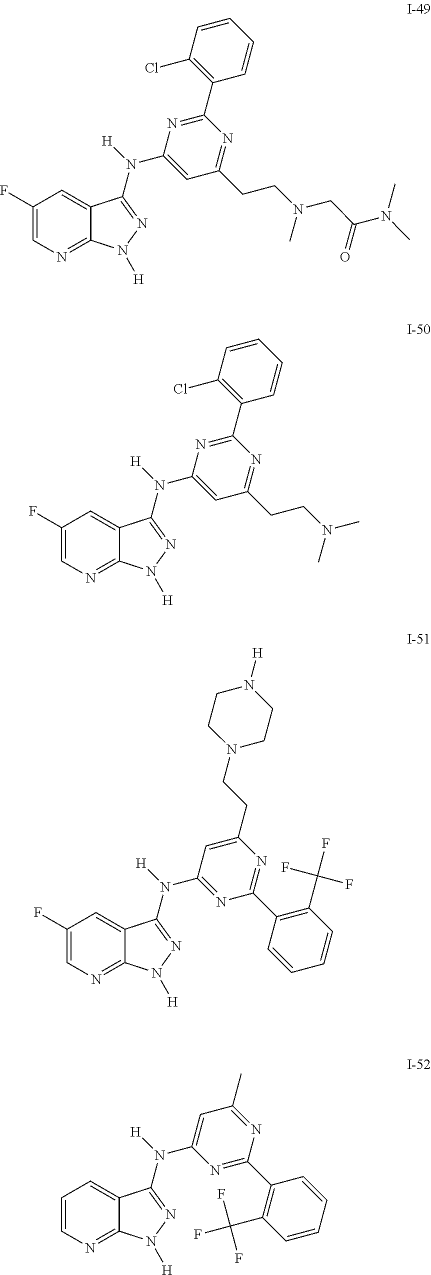 [chemical structure]
